(19) United States
(12) Patent Application Publication
(10) Pub. No.: US 2021/0327533 A1
(43) Pub. Date: Oct. 21, 2021

(54) METHODS FOR GENERATING BROADLY REACTIVE, PAN-EPITOPIC IMMUNOGENS, COMPOSITIONS AND METHODS OF USE THEREOF

(71) Applicant: University of Georgia Research Foundation, Athens, GA (US)

(72) Inventors: James Daniel Allen, Athens, GA (US); Ted Milburn Ross, Athens, GA (US); Terianne Maiko Wong, Athens, GA (US); Anne Gaelle Bebin Blackwell, Athens, GA (US); Ivette Ariela Nunez, Athens, GA (US); Zachary Beau Reneer, Athens, GA (US); Ying Huang, Athens, GA (US)

(73) Assignee: University of Georgia Research Foundation, Athens, GA (US)

(21) Appl. No.: 17/260,041

(22) PCT Filed: Jul. 12, 2019
(86) PCT No.: PCT/US2019/041704

§ 371 Date: Jan. 13, 2021

Related U.S. Application Data
(60) Provisional application No. 62/697,818, filed on Jul. 13, 2018.

(51) Int. Cl. G16B 10/00; A61K 39/145; C07K 14/005

(52) U.S. Cl. CPC G16B 10/00; A61K 39/145; C07K 14/005; C12N 2760/16071; C12N 2760/16034; C12N 2760/16023; C12N 2760/16022

(57) ABSTRACT

Provided herein are methods for generating a non-naturally occurring, broadly reactive, pan-epitopic antigen derived from a pathogen, such as a virus, bacterium, and the like, that is immunogenic and is capable of eliciting a broadly reactive immune response, such as a broadly reactive neutralizing antibody response, against the pathogen following introduction into a subject. Also provided is a non-naturally occurring immunogen generated using the methods, and vaccines and compositions comprising the immunogen. Methods of generating an immune response in a subject by administering the immunogen, vaccine, or composition are provided. In particular, the immunogen comprises the hemagglutinin (HA) or neuraminidase (NA) protein of influenza virus strains.

### CROSS-REFERENCE TO RELATED APPLICATION

This application claims priority to and benefit of U.S. Provisional Application No. 62/697,818, filed on Jul. 13, 2018, the entire contents of which are incorporated by reference herein in their entirety.

### BACKGROUND

On a worldwide scale, microbial pathogens cause disease and illness as infectious agents in mammalian and non-mammalian subjects (hosts) following infection. Infectious pathogens include viruses, bacteria, fungi, and protozoans (parasites) of diverse types and subtypes. In particular, bacterial and viral microorganisms express surface proteins or peptides, (called antigens), which comprise multiple, different epitopes, or antigenic determinants, against which specific antibodies, such as neutralizing antibodies, are generated and to which these antibodies bind. A protein or peptide antigen derived from a pathogenic organism, e.g., a bacterial or viral antigen, is an immunogen and is immunogenic if a susceptible subject is capable of generating an immune response against the antigen following infection. The subject's immune response typically involves the production of antibodies that bind to epitopes of the immunogen and neutralize (clear) the pathogen. The immune response of the subject can also involve the production of T-cells and other immune cells that act in concert with the production of antibodies to destroy the pathogen. Because disease and illness and their symptoms can be severe, and even lead to death, in those who are infected with pathogenic organisms, there is a medical need for better and longer-lasting immunogenic compositions, such as vaccines, that generate potent immune responses and reduce or eliminate disease and symptoms thereof in the subject, either before or after infection.

Influenza virus infection is the most important cause of medically attended acute respiratory illness each year and imposes substantial morbidity, mortality and economic burdens in the United States. Shortly before the 2009 influenza pandemic, influenza-associated mortality accounted for over 611,000 years of life lost annually with an estimated cost to society of $87 billion each year. The U.S. Centers for Disease Control and Prevention estimated in 2017 that the seasonal flu vaccine was only 42% effective. This limited effectiveness was due to a mutation that occurred in the influenza A H3N2 vaccine strain that causes flu in infected individuals. In addition, cases of flu caused by influenza B viruses have risen in the 2017 to 2018 time period. Given that a bad flu season can kill on the order of 50,000 people in the United States alone, new and improved methods for generating immunogens and vaccines that provide broad protection against viruses, particularly, influenza A and influenza B virus strains in present and future circulation, are urgently required.

### SUMMARY OF THE DISCLOSURE

As described below, methods of generating non-naturally occurring, broadly reactive antigens and antigen sequences derived from disease-causing pathogens are provided. The antigens are potent immunogens that can elicit a broadly reactive immune response against the pathogens, such as viral pathogens, e.g., Influenza (flu) viruses, or viruses such as Chikungunya, Dengue, Foot and Mouth disease, Zika, or Rift Valley Fever, or against other types of pathogens, particularly, microorganisms such as bacteria, fungi, protozoans, prions and the like in a subject. As referred to herein, the pathogen-derived antigens or antigen sequences that are generated by the methods and that elicit an immune response in a subject are immunogens. These pathogen-derived (e.g. virus) immunogens are termed broadly reactive and pan-epitopic, because they elicit the production of broadly reactive antibodies that are directed against pathogens (e.g., different subtypes or strains of viruses) having both sequence similarity and variability, and a diversity of epitopes (antigenic determinants) in their antigens and the sequences thereof, such as the HA or NA antigens of influenza virus.

There are four different types of influenza viruses, three of which (influenza A, B, and C) infect people. Of those three infectious viruses, the influenza A and B subsets are the most common types, and each of these subsets develops different strains or subtypes. Influenza A and B viruses routinely spread in humans and cause seasonal flu epidemics. By way of nonlimiting example, the H1N1, H2N2, and H5N1 strains are subtypes of influenza A that typically cause severe flu disease and that adapt to evade being eradicated by constantly changing their surface proteins, such as the hemagglutinin (HA) protein. Strains of influenza virus have been particularly problematic to treat because of unusually high rate of mutation and an inability to generate vaccines that were effective against the relatively rapid changes that occurred in the HA surface protein.

In an embodiment, the antigen (antigen sequence) that is immunogenic is derived from influenza A virus. In certain embodiments, the immunogen is derived from an H1, H5, H7, or H9 influenza virus strain or type, or other influenza virus strain or type. In an embodiment, the immunogen is derived from influenza B virus. In an embodiment, the antigen is a structural protein of the virus. In a particular embodiment, the influenza antigen is hemagglutinin (HA). In another particular embodiment, the influenza antigen is neuraminidase (NA). In other embodiments, the immunogenic antigen is derived from a non-influenza virus. In still other embodiments, the immunogenic antigen is derived from a bacterium or fungus. In an embodiment, the broadly reactive antigens generated by the methods are potent immunogens that can elicit a broadly reactive immune response against antigens of present and future pathogens.

Also provided are immunogenic compositions that contain the broadly reactive, pathogen-derived antigens, e.g., antigen proteins, polypeptides, or peptides, including vaccines (e.g., polypeptide or polynucleotide products), that induce an immune response directed against the antigen in a subject; and methods of using the immunogens to induce an immune response in a subject. In certain embodiments, the antigens are surface proteins derived from a pathogen such as a virus or bacterium. In certain embodiments, the antigens are surface proteins derived from influenza virus. In certain other embodiments, the antigens are HA or NA proteins derived from influenza A or influenza B type viruses. In a particular embodiment, the antigen is an HA protein (or HA1 or HA2 protein), or NA protein of the H1, H5, H7, or H9 types of influenza virus. Methods of using the immunogens to induce an immune response in a subject are also provided.

In an aspect, a method of generating a non-naturally occurring, pan-epitopic immunogen capable of generating an immune response in a subject is provided, in which the method comprises (a) generating a phylogenetic tree comprising full length related antigen sequences derived from one or more pathogens or pathogen strains; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) generating a phylogenetic tree comprising the secondary sequences, wherein branches of the tree are combined if the substitution rate per amino acid site distance is less than about 0.001 to produce a plurality of tertiary sequences; (g) generating a quaternary backbone sequence comprising amino acids that are conserved or identical among the tertiary sequences; and (h) generating a non-naturally occurring, pan-epitopic immunogen by incorporating secondary sequences from step (e), wherein the selected sequences are derived from the most recent time period.

In another aspect, a method of generating a non-naturally occurring, pan-epitopic immunogen capable of generating an immune response against present and future pathogens in a subject is provided, in which the method comprises (a) generating a phylogenetic tree comprising full length related antigen sequences derived from one or more pathogen strains, wherein each pathogenic strain is present in two or more selected geographic regions over one or more selected periods of time; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) generating a phylogenetic tree comprising the secondary sequences, wherein branches of the tree are combined if the substitution rate per amino acid site distance is less than about 0.001 to produce a plurality of tertiary sequences; (g) generating a quaternary backbone sequence comprising amino acids that are conserved or identical among the tertiary sequences; and (h) generating a non-naturally occurring, pan-epitopic immunogen by incorporating secondary sequences from step (e), wherein the selected sequences are derived from the most recent time period.

In an embodiment of the methods in the foregoing aspects, the pathogen is a microorganism such as a virus, bacterium, fungus, protozoan, prion, and the like. In a certain embodiment of the methods in the foregoing aspects, the pathogen is a retrovirus or DNA virus. In a certain embodiment of the methods in the foregoing aspects, the pathogen is a virus selected from Chikungunya, Foot and Mouth disease, Influenza virus, Zika, Rift Valley Fever Virus, and the like. In a particular embodiment of the methods in the foregoing aspects, the pathogen is Influenza virus. In another embodiment of the methods of the foregoing aspects, the virus is present in the Northern or Southern hemispheres. In certain embodiments of the methods in the foregoing aspects, the virus is present in one or more flu seasons. In certain embodiments of the methods in the foregoing aspects, the pathogen is a Gram-positive or Gram-negative bacterium.

In another aspect, a method of generating a non-naturally occurring, pan-epitopic immunogen capable of generating an immune response against present and future influenza virus strains in a subject is provided, in which the method comprises (a) generating a phylogenetic tree comprising full length related antigen sequences derived from one or more influenza virus strains, wherein each influenza virus strain is present in two or more selected geographic regions over one or more flu seasons; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) generating a phylogenetic tree comprising the secondary sequences, wherein branches of the tree are combined if the substitution rate per amino acid site distance is less than about 0.001 to produce a plurality of tertiary sequences; (g) generating a quaternary backbone sequence comprising amino acids that are conserved or identical among the tertiary sequences; and (h) generating a non-naturally occurring, pan-epitopic immunogen by incorporating secondary sequences from step (e), wherein the selected sequences are derived from the most recent time period.

In another aspect, a method of generating a non-naturally occurring, pan-epitopic immunogen capable of generating an immune response against present and future Influenza H5 strains in a subject is provided, in which the method comprises (a) generating a phylogenetic tree comprising full length related antigen sequences derived from two or more H5 clades, or two or more H5 species, present in two or more selected geographic regions over one or more flu seasons; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) generating a phylogenetic tree comprising the secondary sequences, wherein branches of the tree are combined if the substitution rate per amino acid site distance is less than about 0.001 to produce a plurality of tertiary sequences; (g) generating a quaternary backbone sequence comprising amino acids that are conserved or identical among the tertiary sequences; and (h) generating a non-naturally occurring, pan-epitopic immunogen by incorporating secondary sequences from step (e), wherein the selected sequences are derived from the most recent time period.

In another aspect, a method of generating a non-naturally occurring, pan-epitopic immunogen capable of generating an immune response against present and future influenza H1 or H2 strains in a subject is provided, in which the method comprises (a) generating a phylogenetic tree comprising full length related antigen sequences derived from H1 or H2 strains present during two or more consecutive flu seasons; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) generating a phylogenetic tree comprising the secondary sequences, wherein branches of the tree are combined if the substitution rate per amino acid site distance is less than about 0.001 to produce a plurality of tertiary sequences; (g) generating a quaternary backbone sequence comprising amino acids that are conserved or identical among the tertiary sequences; and (h) generating a non-naturally occurring, pan-epitopic immunogen by incorporating secondary sequences from step (e), wherein the selected sequences are derived from the most recent time period.

In an embodiment of the methods of the foregoing aspects, in step (h), the most recent time period is a six-month time period. In an embodiment of the foregoing methods, the phylogenetic tree comprises sequences derived from influenza strains present during the past 1-100 years. In another embodiment of the foregoing methods, the phylogenetic tree comprises sequences derived from influenza strains present during the past 5, 10, 20, 30, 40, 50, or 100 years. In an embodiment of the methods of the foregoing aspects, the antigen sequences are influenza virus HA, HA1, HA2, or NA antigen sequences.

In yet another aspect, a method of generating a non-naturally occurring, pan-epitopic immunogen capable of generating an immune response against present and future Dengue virus strains in a subject is provided, in which the method comprises (a) generating a phylogenetic tree comprising full length related antigen sequences derived from Dengue strains present in the Americas or Asia during a selected period of time; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) generating a phylogenetic tree comprising the secondary sequences, wherein branches of the tree are combined if the substitution rate per amino acid site distance is less than about 0.001 to produce a plurality of tertiary sequences; (g) generating a quaternary backbone sequence comprising amino acids that are conserved or identical among the tertiary sequences; and (h) generating a non-naturally occurring, pan-epitopic immunogen by incorporating secondary sequences from step (e), wherein the selected sequences are derived from the most recent time period. In an embodiment of the method, in step (h), the most recent time period is a six-month time period.

In an embodiment of the methods in any of the foregoing aspects, step (d) and step (e) are optional.

In an embodiment of the methods in any of the foregoing aspects, steps (a)-(c) are repeated two or more times.

In an embodiment of the methods in any of the foregoing aspects, the method further comprises repeating steps (b) and (c) two or more times prior to step (d).

In an embodiment of the methods in any of the foregoing aspects, the method further comprises repeating steps (e) and (f) two or more times.

In an embodiment of the methods in any of the foregoing aspects, three or more clusters of antigen sequences are identified in step (b). In a particular embodiment of the methods, 5 or 15 clusters of antigen sequences are identified in step (b).

In another of its aspects, the above-described methods of generating a non-naturally occurring, pan-epitopic immunogen capable of generating an immune response against a pathogen, such as a virus, for example, influenza virus (e.g., H1, H5, H7, H9, or other strains of influenza) in a subject, further comprise generating at least 2 secondary antigen sequences for a disease season of time based on the phylogenetic tree sequences generated in step (f); adding the at least 2 secondary sequences to a backbone sequence based on clustered antigen sequences in the season; eliminating from the backbone sequence older secondary antigen sequences produced from a predetermined disease season in a geographic location; and generating a pan-epitopic immunogen by equally weighting multiple secondary antigen sequences.

In another of its aspects, the above-described methods of generating a non-naturally occurring, pan-epitopic immunogen capable of generating an immune response against a pathogen, such as a virus, for example, influenza virus (e.g., H1, H5, H7, H9, or other strains of influenza) in a subject, further comprise generating at least 2 secondary antigen sequences for a disease season of time based on the phylogenetic tree sequences generated in step (f); adding the at least 2 secondary sequences to a backbone sequence based on clustered antigen sequences in the season; retaining in the backbone sequence antigen sequences produced from a predetermined disease season in a geographic location to include an additional season; and generating a pan-epitopic immunogen by equally weighting multiple secondary antigen sequences.

In an embodiment of the methods in any of the foregoing aspects, the results or output generated by the practice of the methods, such as primary, secondary and tertiary sequences, sequence clusters, sequence alignments, phylogenetic trees, or any combinations thereof, are displayed in a visual form, such as displaying the results or output on a display device. In certain embodiments, the display device is a desktop computer, a laptop computer, a hand-held computer, a smart phone, a cellular telephone, a tablet computer, or a personal digital assistant.

It will be understood that the methods described herein generate a non-naturally occurring, broadly reactive, pan-epitopic immunogen, which may be referred to interchangeably herein, as a “non-naturally occurring immunogen,” a “broadly reactive immunogen,” or a “pan-epitopic immunogen,” for simplicity.

In an embodiment of the methods of any of the foregoing aspects, the immunogen generated by the practice of the method is isolated and/or purified. In an embodiment of the methods, the immunogen is formulated for administration to a subject in need thereof. In an embodiment of the methods, the immunogen or a composition thereof is administered to a subject in need thereof in an effective amount to elicit an immune response in the subject.

In an embodiment of the methods in any of the foregoing aspects, the immune response elicits neutralizing antibodies. In an embodiment, the immune response further elicits a T-lymphocyte response. In an embodiment, the immune response is prophylactic or therapeutic.

In another aspect, a non-naturally occurring immunogen is provided, wherein the non-naturally occurring immunogen is generated using the method as described in any of the foregoing aspects.

In another aspect, a vaccine comprising the non-naturally occurring immunogen generated using the method as described in any of the foregoing aspects is provided.

In another aspect, a virus-like particle (VLP) comprising the non-naturally occurring immunogen, or sequence thereof, generated using the methods as described in any of the foregoing aspects is provided. In an embodiment, a vaccine comprising the VLP is provided. In another aspect, a composition comprising the immunogen, vaccine, or VLP of any of the foregoing aspects and a pharmaceutically acceptable carrier is provided. In an embodiment, the composition further comprises an adjuvant.

In another aspect, a method of generating an immune response in a subject is provided, in which the method comprises administering to the subject an effective amount of a non-naturally occurring immunogen generated using the method according to any one of the foregoing aspects.

In another aspect, the method of generating a non-naturally occurring pan-epitopic immunogen capable of generating an immune response against present and future H5 strains provides an immunogen comprising an amino acid sequence that is at least 95% or at least 98% identical to an amino acid sequence of a hemagglutinin (HA) antigen as set forth in FIG. 3. In an embodiment, the immunogen generated by the method comprises an amino acid sequence of an HA antigen as set forth in FIG. 3. In an embodiment, the immunogen generated by the method is provided as a vaccine. In an embodiment, the vaccine is provided as a VLP. In an embodiment, the immunogen generated by the method is provided as a composition comprising a pharmaceutically acceptable carrier, diluent, or excipient. In an embodiment, the immunogen, vaccine, or VLP is used in a method of generating an immune response in a subject, in which the method comprises administering to the subject an effective amount of the immunogen, vaccine, or VLP.

In another aspect, the method of generating a non-naturally occurring pan-epitopic immunogen capable of generating an immune response against present and future influenza virus strains provides an immunogen comprising an amino acid sequence that is at least 95% or at least 98% identical to an amino acid sequence of a neuraminidase (NA) antigen as set forth in FIG. 4, FIG. 5, or FIGS. 6A and 6B. In an embodiment, the immunogen generated by the method comprises an amino acid sequence of an NA antigen as set forth in FIG. 4, FIG. 5, or FIGS. 6A and 6B. In an embodiment, the immunogen generated by the method is provided as an immunogenic composition or a vaccine. In an embodiment, the immunogenic composition a vaccine is provided as a VLP. In an embodiment of any of the foregoing aspects, the immunogen, vaccine, VLP, or composition is used in a method of generating an immune response in a subject, in which the method comprises administering to the subject an effective amount of the immunogen, vaccine, VLP, or immunogenic composition. In an embodiment, an adjuvant is concomitantly administered to the subject.

In an embodiment of the method of any of the above aspects, the full length related antigen sequence derived from the first pathogen or virus strains selected is deleted from the analysis each time a pathogen or virus strain selected from a later time period or disease season is added to the analysis. In an embodiment, the disease season is a flu season.

In an embodiment of the method of any of the above aspects, steps a-g are repeated a plurality of times, such that in each repetition full length related antigen sequences are derived from a set of pathogen or virus strains present during a time period or disease season that is one time period or disease season later in time than the time periods or disease seasons of the first set of pathogen or virus strains selected for analysis. In an embodiment, the disease season is a flu season.

In an embodiment of the method of any of the above aspects, steps a-g are repeated a plurality of times, such that in each repetition a full length related antigen sequence is derived from a set of pathogen or virus strains present during consecutive time periods or disease seasons up to the most recent time period or disease season selected for analysis. In an embodiment, the disease season is a flu season.

In another aspect, a method of generating a non-naturally occurring, pan-epitopic immunogen capable of generating an immune response in a subject is provided, in which the method comprises (a) generating a phylogenetic tree comprising full length related antigen sequences derived from one or more pathogens or pathogen strains present within a six-month time period; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) repeating steps (a)-(e) until secondary sequences from a series of recent consecutive six-month time periods have been selected over a preselected total time period; (g) generating a phylogenetic tree comprising the secondary sequences, wherein branches of the tree are combined if the substitution rate per amino acid site distance is less than about 0.001 to produce a plurality of tertiary sequences; (h) generating a quaternary backbone sequence comprising amino acids that are conserved or identical among the tertiary sequences; and (i) generating a non-naturally occurring, pan-epitopic immunogen by incorporating secondary sequences from step (e) into the quaternary backbone sequence, wherein: (i) secondary sequences from the most recent six-month time period are incorporated into the backbone sequence and secondary sequences from the oldest six-month time period are eliminated from the backbone sequence, thereby producing a sequence comprising multiple secondary sequences spanning the preselected total time period; or (ii) secondary sequences from the most recent six-month time period and from the oldest six-month time period are incorporated into the backbone sequence, thereby producing a sequence comprising multiple secondary sequences spanning the preselected total time period.

In another aspect, a method of generating a non-naturally occurring pan-epitopic immunogen capable of generating an immune response against present and future pathogens in a subject is provided, in which the method comprises (a) generating a phylogenetic tree comprising full length related antigen sequences derived from one or more pathogen strains, wherein each pathogenic strain is present in two or more selected geographic regions over one or more selected periods of time within a six-month time period; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) repeating steps (a)-(e) until secondary sequences from a series of recent consecutive six-month time periods have been selected over a preselected total time period; (g) generating a phylogenetic tree comprising the secondary sequences, wherein branches of the tree are combined if the substitution rate per amino acid site distance is less than about 0.001 to produce a plurality of tertiary sequences; (h) generating a quaternary backbone sequence comprising amino acids that are conserved or identical among the tertiary sequences; and (i) generating a non-naturally occurring, pan-epitopic immunogen by incorporating secondary sequences from step (e) into the quaternary backbone sequence, wherein: (i) secondary sequences from the most recent six-month time period are incorporated into the backbone sequence and secondary sequences from the oldest six-month time period are eliminated from the backbone sequence, thereby producing a sequence comprising multiple secondary sequences spanning the preselected total time period; or (ii) secondary sequences from the most recent six-month time period and from the oldest six-month time period are incorporated into the backbone sequence, thereby producing a sequence comprising multiple secondary sequences spanning the preselected total time period.

In another aspect, a method of generating a non-naturally occurring, pan-epitopic immunogen capable of generating an immune response against present and future influenza virus strains in a subject is provided, the method comprising (a) generating a phylogenetic tree comprising full length related antigen sequences derived from one or more influenza virus strains, wherein each influenza virus strain is present in two or more selected geographic regions over one or more flu seasons within a six-month time period; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) repeating steps (a)-(e) until secondary sequences from a series of recent consecutive six-month time periods have been selected over a preselected total time period; (g) generating a phylogenetic tree comprising the secondary sequences, wherein branches of the tree are combined if the substitution rate per amino acid site distance is less than about 0.001 to produce a plurality of tertiary sequences; (h) generating a quaternary backbone sequence comprising amino acids that are conserved or identical among the tertiary sequences; and (i) generating a non-naturally occurring, pan-epitopic immunogen by incorporating secondary sequences from step (e) into the quaternary backbone sequence, wherein: (i) secondary sequences from the most recent six-month time period are incorporated into the backbone sequence and secondary sequences from the oldest six-month time period are eliminated from the backbone sequence, thereby producing a sequence comprising multiple secondary sequences spanning the preselected total time period; or (ii) secondary sequences from the most recent six-month time period and from the oldest six-month time period are incorporated into the backbone sequence, thereby producing a sequence comprising multiple secondary sequences spanning the preselected total time period.

In another aspect, a method of generating A method of generating a non-naturally occurring pan-epitopic immunogen capable of generating an immune response against present and future influenza H5 strains, the method comprising (a) generating a phylogenetic tree comprising full length related antigen sequences derived from two or more H5 clades, or two or more H5 species, present in two or more selected geographic regions over one or more flu seasons within a six-month time period; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) repeating steps (a)-(e) until secondary sequences from a series of recent consecutive six-month time periods have been selected over a preselected total time period; (g) generating a phylogenetic tree comprising the secondary sequences, wherein branches of the tree are combined if the substitution rate per amino acid site distance is less than about 0.001 to produce a plurality of tertiary sequences; (h) generating a quaternary backbone sequence comprising amino acids that are conserved or identical among the tertiary sequences; and (i) generating a non-naturally occurring, pan-epitopic immunogen by incorporating secondary sequences from step (e) into the quaternary backbone sequence, wherein: (i) secondary sequences from the most recent six-month time period are incorporated into the backbone sequence and secondary sequences from the oldest six-month time period are eliminated from the backbone sequence, thereby producing a sequence comprising multiple secondary sequences spanning the preselected total time period; or (ii) secondary sequences from the most recent six-month time period and from the oldest six-month time period are incorporated into the backbone sequence, thereby producing a sequence comprising multiple secondary sequences spanning the preselected total time period.

In another aspect, method of generating a non-naturally occurring, pan-epitopic immunogen capable of generating an immune response against present and future influenza H1 or H2 strains in a subject is provided, in which the method comprises (a) generating a phylogenetic tree comprising full length related antigen sequences derived from H1 or H2 strains present during two or more consecutive flu seasons within a six-month time period; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) repeating steps (a)-(e) until secondary sequences from a series of recent consecutive six-month time periods have been selected over a preselected total time period; (g) generating a phylogenetic tree comprising the secondary sequences, wherein branches of the tree are combined if the substitution rate per amino acid site distance is less than about 0.001 to produce a plurality of tertiary sequences; (h) generating a quaternary backbone sequence comprising amino acids that are conserved or identical among the tertiary sequences; and (i) generating a non-naturally occurring, pan-epitopic immunogen by incorporating secondary sequences from step (e) into the quaternary backbone sequence, wherein: (i) secondary sequences from the most recent six-month time period are incorporated into the backbone sequence and secondary sequences from the oldest six-month time period are eliminated from the backbone sequence, thereby producing a sequence comprising multiple secondary sequences spanning the preselected total time period; or (ii) secondary sequences from the most recent six-month time period and from the oldest six-month time period are incorporated into the backbone sequence, thereby producing a sequence comprising multiple secondary sequences spanning the preselected total time period.

In another aspect, method of generating a non-naturally occurring, pan-epitopic immunogen capable of generating an immune response against present and future Dengue virus strains in a subject is provided, in which the method comprises (a) generating a phylogenetic tree comprising full length related antigen sequences derived from Dengue strains present in the Americas or Asia during a selected period of time comprising a six month time period; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; (f) repeating steps (a)-(e) until secondary sequences from a series of recent consecutive six-month time periods have been selected over a preselected total time period; (g) generating a phylogenetic tree comprising the secondary sequences, wherein branches of the tree are combined if the substitution rate per amino acid site distance is less than about 0.001 to produce a plurality of tertiary sequences; (h) generating a quaternary backbone sequence comprising amino acids that are conserved or identical among the tertiary sequences; and (i) generating a non-naturally occurring, pan-epitopic immunogen by incorporating secondary sequences from step (e) into the quaternary backbone sequence, wherein: (i) secondary sequences from the most recent six-month time period are incorporated into the backbone sequence and secondary sequences from the oldest six-month time period are eliminated from the backbone sequence, thereby producing a sequence comprising multiple secondary sequences spanning the preselected total time period; or (ii) secondary sequences from the most recent six-month time period and from the oldest six-month time period are incorporated into the backbone sequence, thereby producing a sequence comprising multiple secondary sequences spanning the preselected total time period.

In an embodiment of the methods in any of the above-delineated aspects, the preselected total time period is 2.5 years.

### Definitions

Unless defined otherwise, all technical and scientific terms used herein have the meaning commonly understood by a person skilled in the art to which this invention pertains or relates. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “adjuvant” is meant a substance or vehicle that non-specifically enhances the immune response to an antigen. Adjuvants may include a suspension of minerals (e.g., alum, aluminum hydroxide, or phosphate) on which antigen is adsorbed; or water-in-oil emulsion in which antigen solution is emulsified in mineral oil (e.g., Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (see, e.g., U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include, without limitation, interleukin-1 (IL-2), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), interferon-gamma (IFN-γ), granulocyte-colony stimulation factor (G-CSF), lymphocyte function-associated antigen 3 (LFA-3, also called CD58), cluster of differentiation antigen 72 (CD72), (a negative regulator of B cell responsiveness), peripheral membrane protein, B7-1 (B7-1, also called CD80), peripheral membrane protein, B7-2 (B7-2, also called CD86), the TNF ligand superfamily member 4 ligand (OX40L) or the type 2 transmembrane glycoprotein receptor belonging to the TNF superfamily (4-1BBL) By “administer” is meant giving, supplying, dispensing a composition, agent, therapeutic and the like to a subject, or applying or bringing the composition and the like into contact with the subject. Administering or administration may be accomplished by any of a number of routes, such as, for example, without limitation, topical, oral, subcutaneous, intramuscular, intraperitoneal, intravenous (IV), (injection), intrathecal, intramuscular, dermal, intradermal, intracranial, inhalation, rectal, intravaginal, or intraocular.

By “agent” is meant any small molecule chemical compound, antibody, nucleic acid molecule, peptide, polypeptide, or fragments thereof.

By “alteration” is meant a change (increase or decrease) in the expression levels or activity of a gene or polypeptide as detected by standard art known methods such as those described herein. As used herein, an alteration includes a 5% change in expression levels, a 10% change in expression levels, preferably a 25% change, more preferably a 40% change, and most preferably a 50% or greater change in expression levels.”

By “ameliorate” is meant decrease, reduce, diminish, suppress, attenuate, arrest, or stabilize the development or progression of a disease or pathological condition.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antibody” is meant an immunoglobulin (Ig) molecule produced by B lymphoid cells and having a specific amino acid sequence. Antibodies are evoked or elicited in subjects (humans or other animals or mammals) following exposure to a specific antigen (immunogen). A subject capable of generating antibodies/immunoglobulin (i.e., an immune response) directed against a specific antigen/immunogen is said to be immunocompetent. Antibodies are characterized by reacting specifically with (e.g., binding to) an antigen or immunogen in some demonstrable way, antibody and antigen/immunogen each being defined in terms of the other.

“Eliciting an antibody response” refers to the ability of an antigen, immunogen or other molecule to induce the production of antibodies. Antibodies are of different classes, e.g., IgM, IgG, IgA, IgE, IgD and subtypes or subclasses, e.g., IgG1, IgG2, IgG2a, IgG2b, IgG3, IgG4. An antibody/immunoglobulin response elicited in a subject can neutralize a pathogenic (e.g., infectious or disease-causing) agent by binding to epitopes (antigenic determinants) on the agent and blocking or inhibiting the activity of the agent, and/or by forming a binding complex with the agent that is cleared from the system (or body) of the subject, e.g., via the liver.

As used herein, “broadly reactive” means that an immune response is elicited against a pathogen-derived antigen protein (e.g., a virus protein sequence, such as HA or NA) in a subject that is sufficient to block, inhibit, impede, neutralize, or prevent infection of a broad range of related pathogens (such as most or all influenza viruses within a specific subtype).

By “antigen” is meant a compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. In some embodiments of the disclosed compositions and methods, the antigen is an influenza hemagglutinin (HA) protein. In many cases, an antigen that elicits or stimulates an immune response in a subject is termed an “immunogen.”

The term “antigenic drift” refers to a mechanism for variation in organisms or microorganisms such as viruses that involves the accumulation of mutations within the genes that code for antibody-binding sites (also called antigenic determinants or epitopes). This process results in a new strain of virus/virus particles that is not inhibited or blocked as effectively by antibodies that were originally generated against the antigens of virus strains prior to mutation, thus allowing the virus to spread more easily throughout a partially immune population. By way of example, antigenic drift occurs in both influenza A and influenza B viruses.

In the context of a live virus, the term “attenuated” reflects a virus that is attenuated if its ability to infect a cell or subject and/or its ability to produce disease is reduced (for example, diminished, abrogated, or eliminated) compared to the ability of a wild-type virus to produce disease in the subject. Typically, an attenuated virus retains at least some capacity to elicit an immune response following administration to an immunocompetent subject. In some cases, an attenuated virus can elicit a protective immune response without causing any signs or symptoms of infection. In some embodiments, the ability of an attenuated virus to cause disease or pathology in a subject is reduced at least about or equal to 5%, or at least about or equal to 10%, or at least about or equal to 25%, at least about or equal to 50%, at least about or equal to 75%, or at least about or equal to 80%, or at least about or equal to 85%, or at least about or equal to 90%, or at least about or equal to 95%, or greater, relative to the ability of a wild-type virus to cause disease or pathology in the subject.

The term “clade” refers to the different categorizations (often called subtypes) of the known influenza viruses, such as the influenza A H1, H5, H5N1, H7, and H9 viruses, or viruses related thereto. By way of nonlimiting example, viruses in a clade, e.g., an H5N1 clade, are genetically related, but do not share the exact viral genome. There are at least ten different clades of H5N1 virus subtypes designated in the art: clade 0 clade 1, clade 2, clade 3, clade 4, clade 5, clade 6, clade 7, clade 8 and clade 9 (Abdel-Ghafar et al., N Engl J Med 358:261-273, 2008). Clade 2 is further divided into sub-clades (including clade 2.1, clade 2.2, clade 2.3, clade 2.4 and clade 2.5).

A “codon-optimized” nucleic acid refers to a nucleic acid sequence that has been altered such that the codons are optimal for expression in a particular system (such as a particular species of group of species). For example, a nucleic acid sequence can be optimized for expression in mammalian cells. Codon optimization does not alter the amino acid sequence of the encoded protein.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of an analyte, compound, agent, or substance to be detected. By “detectable label” is meant a composition that, when linked to a molecule of interest, renders the latter detectable, e.g., via spectroscopic, photochemical, biochemical, immunochemical, or chemical means.

Nonlimiting examples of useful detectable labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. Examples of diseases include those caused by microorganisms, such as viral infections, bacterial infections and the symptoms and adverse effects that are caused by infection of the body with these organisms. Examples of diseases are infections and symptoms thereof caused by viruses such as influenza (types A, B and C), Dengue, Foot and Mouth, Chikungunya, Zika, or Rift Valley Fever viruses.

By “effective amount” is meant the amount of an active therapeutic agent, composition, compound, biologic (e.g., a vaccine or therapeutic peptide, polypeptide, or polynucleotide) required to ameliorate, reduce, improve, abrogate, diminish, or eliminate the symptoms and/or effects of a disease, condition, or pathology relative to an untreated patient. In one embodiment, an effective amount is the amount of an antigen required to elicit an immune response. The effective amount of an immunogen or a composition comprising the immunogen, as used to practice the methods of therapeutic treatment of a disease, condition, or pathology, varies depending upon the manner of administration, the age, body weight, and general health of the subject. Ultimately, the attending physician or veterinarian will decide the appropriate amount and dosage regimen. Such amount is referred to as an “effective” amount.

A “therapeutically effective amount” refers to a quantity of a specified agent sufficient to achieve a desired effect in a subject being treated with that agent. For example, this may be the amount of an influenza virus immunogen or vaccine useful for eliciting an immune response in a subject and/or for preventing infection by influenza virus. Ideally, in the context of the present disclosure, a therapeutically effective amount of an influenza vaccine or immunogenic composition is an amount sufficient to increase resistance to, prevent, ameliorate, reduce, and/or treat infection caused by influenza virus in a subject without causing a substantial cytotoxic effect in the subject. The effective amount of an influenza vaccine useful for increasing resistance to, preventing, ameliorating, reducing, and/or treating infection in a subject depends on, for example, the subject being treated, the manner of administration of the therapeutic composition and other factors, as noted supra.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. A portion or fragment of a polypeptide may be a peptide. In the case of an antibody or immunoglobulin fragment, the fragment typically binds to the target antigen.

By “fusion protein” is meant a protein generated by expression of a nucleic acid (polynucleotide) sequence engineered from nucleic acid sequences encoding at least a portion of two different (heterologous) proteins or peptides. To create a fusion protein, the nucleic acid sequences must be in the same reading frame and contain no internal stop codons. For example, a fusion protein includes an influenza HA protein fused to a heterologous protein.

By “genetic vaccine” is meant an immunogenic composition comprising a polynucleotide encoding an antigen.”

The terms “geographical location or geographical region” refers to preselected divisions of geographical areas of the earth, for example, by continent or other preselected territory or subdivision (e.g., the Middle East, which spans more than one continent). Examples of different geographical regions include countries (e.g., Turkey, Egypt, Iraq, Azerbaijan, China, United States); continents (e.g., Asia, Europe, North America, South America, Oceania, Africa); recognized geopolitical subdivisions (such as the Middle East); or hemispheres of the world (e.g., Northern, Southern, Eastern, or Western hemispheres).

The term “Hemagglutinin (HA)” refers to a surface glycoprotein expressed by an influenza virus. HA mediates binding of the virus particle to a host cell and subsequent entry of the virus into the host cell. The nucleotide and amino acid sequences of numerous influenza HA proteins are known in the art and are publically available, such as those deposited with GenBank. By way of nonlimiting example, a list of GenBank Accession Nos. of H5N1 HA sequences may be found in US Patent Application Publication US 2015/0030628. HA (along with neuraminidase (NA)) is one of the two major influenza virus antigenic proteins having antigenic determinants (epitopes) that are recognized and bound by antibodies/immunoglobulins.

“Hybridization” means hydrogen bonding, which may be Watson-Crick, Hoogsteen, or reversed Hoogsteen hydrogen bonding, between complementary nucleobases. For example, in DNA, adenine and thymine, and cytosine and guanine, are, respectively, complementary nucleobases that pair through the formation of hydrogen bonds.

The term “immune response” is meant any response mediated by an immunoresponsive cell. In one example of an immune response, leukocytes are recruited to carry out a variety of different specific functions in response to exposure to an antigen (e.g., a foreign entity). Immune responses are multifactorial processes that differ depending on the type of cells involved. Immune responses include cell-mediated responses (e.g., T cell responses), humoral responses (B cell/antibody responses), innate responses and combinations thereof.

By “immunogen” is meant a compound, composition, or substance which, under appropriate conditions, can elicit or stimulate an immune response, such as the production of antibodies, and/or a T-cell response, in an animal, including compositions that are injected or absorbed into an animal. As used herein, an “immunogenic composition” is a composition comprising an immunogen (such as an HA or NA polypeptide or a polynucleotide encoding such immunogen) or a vaccine comprising an HA or NA polypeptide or a polynucleotide encoding such immunogen). As will be appreciated by the skilled person in the art, if administered to a subject in need prior to the subject's contracting disease or experiencing full-blown disease, an immunogenic composition can be prophylactic and result in the subject's eliciting an immune response, e.g., a neutralizing antibody and/or cellular immune response, to protect against disease, or to prevent more severe disease or condition, and/or the symptoms thereof. If administered to a subject in need following the subject's contracting disease, an immunogenic composition can be therapeutic and result in the subject's eliciting an immune response, e.g., a neutralizing antibody and/or cellular immune response, to treat the disease, e.g., by reducing, diminishing, abrogating, ameliorating, or eliminating the disease, and/or the symptoms thereof. In an embodiment, the immune response is a B cell response, which results in the production of antibodies, e.g., neutralizing antibodies, directed against the immunogen or immunogenic composition comprising the antigen or antigen sequence. In a manner similar to the foregoing, in some embodiments, an immunogenic composition or vaccine can be prophylactic. In some embodiments, an immunogenic composition or vaccine can be therapeutic. In an embodiment, the disease is influenza (flu).

By “immunogenic composition” is meant a composition comprising an antigen or antigen sequence, wherein the composition elicits an immune response in an immunized subject.

The term “immunize” (or immunization) refers to rendering a subject protected from a disease or pathology caused by a pathogenic agent, e.g., an infectious disease caused by a virus or bacterium), such as by vaccination.

The term “Influenza virus” refers to a segmented negative-strand RNA virus that belongs to the Orthomyxoviridae family of viruses. There are three types of Influenza viruses: A, B and C. Influenza A viruses infect a wide variety of birds and mammals, including humans, horses, marine mammals, pigs, ferrets, and chickens. In animals, most influenza A viruses cause mild localized infections of the respiratory and intestinal tract. However, highly pathogenic influenza A strains, such as H5N1, H5N2, H5N6, H5N8, H7N9, H9N2, H1N1, H1N2, H2N1, H2N2, H2N3, H7N3, H7N7, H3N2, H3N1, and related viruses, cause systemic infections in poultry in which mortality may reach 100%. H5N1 is also referred to as “avian influenza.”

By “inhibitory nucleic acid” is meant a double-stranded RNA, siRNA, shRNA, or antisense RNA, or a portion thereof, or a mimetic thereof, that when administered to a mammalian cell results in a decrease (e.g., by 5%, 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid, protein, or peptide is purified if it is substantially free of cellular material, debris, non-relevant viral material, or culture medium when produced by recombinant DNA techniques, or of chemical precursors or other chemicals when chemically synthesized. Purity and homogeneity are typically determined using standard purification methods and analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. The term “isolated” also embraces recombinant nucleic acids, proteins or viruses, as well as chemically synthesized nucleic acids or peptides.

By “isolated polynucleotide” is meant a nucleic acid (e.g., a DNA molecule) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 40%, by weight, at least 50%, by weight, at least 60%, by weight, free from the proteins and naturally-occurring organic molecules with which it is naturally associated. Preferably, an isolated polypeptide preparation is at least 75%, more preferably at least 90%, and most preferably, at least 99%, by weight, free from the proteins and naturally-occurring organic molecules with which it is naturally associated. An isolated polypeptide may be obtained, for example, by extraction from a natural source; by expression of a recombinant nucleic acid encoding such a polypeptide; or by chemically synthesizing the protein. Purity can be measured by any standard, appropriate method, for example, column chromatography, polyacrylamide gel electrophoresis, or by HPLC analysis. An isolated polypeptide can refer to broadly active virus immunogen polypeptide generated by the methods described herein.

By “linker” is meant one or more amino acids that serve as a spacer between two polypeptides or peptides of a fusion protein.

By “marker” is meant any protein or polynucleotide having an alteration in expression level or activity that is associated with a disease, condition, pathology, or disorder.

A “Matrix (M1) protein” refers to an influenza virus structural protein found within the viral envelope. M1 is thought to function in assembly and budding of virus following infection of a cell.

The term “Neuraminidase (NA)” refers to an influenza virus membrane glycoprotein. NA is involved in the destruction of the cellular receptor for the viral HA by cleaving terminal sialic acid residues from carbohydrate moieties on the surfaces of infected cells. NA also cleaves sialic acid residues from viral proteins, preventing aggregation of viruses. NA (along with HA) is one of the two major influenza virus antigenic determinants.

As used herein, “obtaining” as in “obtaining an agent” includes synthesizing, isolating, purchasing, or otherwise acquiring the agent.

The term “operably linked” refers to nucleic acid sequences as used herein. By way of example, a first nucleic acid sequence is operably linked to a second nucleic acid sequence when the first nucleic acid sequence is placed in a functional relationship with the second nucleic acid sequence. For instance, a promoter is operably linked to a coding sequence if the promoter affects (allows) the transcription or expression of the coding sequence. Generally, operably linked DNA sequences are contiguous and, where necessary to join two protein-coding regions, are in the same reading frame.

The nucleotide sequence encoding an HA protein generated by the described methods can be optimized for expression in mammalian cells via codon-optimization and RNA optimization (such as to increase RNA stability) using procedures and techniques practiced in the art.

A broadly reactive, pan-epitopic immunogen (or immunogenic antigen), such as influenza hemagglutinin (HA) protein, for eliciting an immune response in a subject is produced from the methods, described herein, of generating a broadly reactive antigen derived from a pathogen, such as an influenza HA protein antigen, which possesses a collective set of strongly immunogenic epitopes (also called antigenic determinants). In the case of influenza HA antigen, the methods described herein are based on focused analysis of populations of similar viral HA proteins to generate a “pan-epitopic” immunogen, such as a vaccine, which elicits a broadly reactive immune response, e.g., a neutralizing antibody response, against a plurality of virus types which express HA proteins on the viral surface, when introduced into a host subject, in particular, a human subject infected with the pathogen. The immunogenic antigen (or vaccine) resulting from the practice of the methods is advantageous for providing an anti-pathogen (e.g., viral) immunogen, such as a vaccine, that elicits a broadly active immune response the pathogen-derived antigen. In the case of influenza, the immunogenic antigen (or vaccine) resulting from the practice of the methods provides an anti-influenza virus immunogen (or a vaccine) that elicits a broadly active immune response against influenza virus HA antigens with antigenic variability and similarity, and treats or protects against infection and disease caused by many different influenza virus types and subtypes.

Similarly, a broadly reactive influenza neuraminidase (NA) protein such as described herein is generated from the methods as described herein that provides an influenza NA antigen that possesses a collective set of strongly immunogenic epitopes (antigenic determinants), based on focused analysis of populations of similar viral NA proteins, i.e., a “pan-epitopic” NA antigen, that is suitable for use as an immunogen, such as a vaccine or VLPs, which elicits a broadly reactive immune response, e.g., a neutralizing antibody response, against a plurality of virus types which express NA proteins on the viral surface. The immunogenic antigen (or vaccine) resulting from the practice of the method is advantageous for providing an anti-pathogen (e.g., viral) immunogen (or a vaccine) that elicits a potent, broadly active immune response against many different influenza virus NA antigens and treats or protects against infection and disease caused by many different influenza virus types and subtypes.

By “open reading frame (ORF)” is meant a series of nucleotide triplets (codons) that code for amino acids without any termination codons. These sequences are usually translatable into a peptide or polypeptide.

As used herein, an influenza virus “outbreak” refers to a collection of virus isolates from within a geographical location (e.g., within a single country) in a given period of time (e.g., in a year).

The term “pharmaceutically acceptable vehicle” refers to conventional carriers (vehicles) and excipients that are physiologically and pharmaceutically acceptable for use, particularly in mammalian, e.g., human, subjects. Such pharmaceutically acceptable vehicles are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic compositions, such as one or more influenza vaccines, and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers may include, for example, pharmaceutical grades of mannitol, lactose, starch, or magnesium stearate, which typically stabilize and/or increase the half-life of a composition or drug. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

By “plasmid” (or “vector”) is meant a circular nucleic acid molecule capable of autonomous replication in a host cell.

By “polypeptide” (or protein) is meant a polymer in which the monomers comprise amino acid residues that are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” also refers to an amino acid that is incorporated into a protein, polypeptide, or peptide.

Conservative amino acid substitutions are those substitutions that, when made, least interfere with the properties of the original protein, that is, the structure and especially the function of the protein is conserved and is not significantly changed by such substitutions. Examples of conservative amino acid substitutions are known in the art, e.g., as set forth in, for example, U.S. Publication No. 2015/0030628. Conservative substitutions generally maintain (a) the structure of the polypeptide backbone in the area of the substitution, for example, as a sheet or helical conformation; (b) the charge or hydrophobicity of the molecule at the target site; and/or (c) the bulk of the side chain

The substitutions that are generally expected to produce the greatest changes in protein properties are non-conservative, for instance, changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

By “parameter” is meant a variable or condition that is assessed or taken into consideration. Examples of parameters taken into consideration in embodiments of the methods include season (time), geography, clade, species or type.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “promoter” is meant an array of nucleic acid control sequences, which direct transcription of a nucleic acid. A promoter includes necessary nucleic acid sequences near the start site of transcription. A promoter also optionally includes distal enhancer or repressor sequence elements. A “constitutive promoter” is a promoter that is continuously active and is not subject to regulation by external signals or molecules. In contrast, the activity of an “inducible promoter” is regulated by an external signal or molecule (for example, a transcription factor). By way of example, a promoter may be a CMV promoter.

As will be appreciated by the skilled practitioner in the art, the term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to routine methods, such as fractionation, chromatography, or electrophoresis, to remove various components of the initial preparation, such as proteins, cellular debris, and other components.

A “recombinant” nucleic acid, protein or virus is one that has a sequence that is not naturally occurring or that has a sequence that is made by an artificial combination of two otherwise separated segments of sequence. Such an artificial combination is often accomplished by chemical synthesis or by the artificial manipulation of isolated segments of nucleic acids, for example, by genetic engineering techniques. A “non-naturally occurring” nucleic acid, protein or virus is one that may be made via recombinant technology, artificial manipulation, or genetic or molecular biological engineering procedures and techniques, such as those commonly practiced in the art.

By “reduces” is meant a negative alteration of at least 5%, 10%, 25%, 30%, 40%, 50%, 75%, 80%, 85%, 90%, 95%, 98%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide, such as a virus polypeptide, peptide, or vaccine product, but which does not substantially recognize and bind other molecules in a sample, for example, a biological sample, which naturally includes a polypeptide, such as a virus polypeptide or peptide.

Nucleic acid molecules useful in the methods described herein include any nucleic acid molecule that encodes a polypeptide as described, or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pairing to form a double-stranded molecule between complementary polynucleotide sequences (e.g., a gene), or portions thereof, under various conditions of stringency. (See, e.g., Wahl, G. M. and S. L. Berger, (1987), Methods Enzymol., 152:399; Kimmel, A. R., (1987), Methods Enzymol. 152:507).

By way of example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, or at least 80% or 85%, or at least or equal to 90%, 95% or even 99% identical at the amino acid level or nucleic acid to the sequence used for comparison.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J. Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

By “subject” is meant an animal, e.g., a mammal, including, but not limited to, a human, a non-human primate, or a non-human mammal, such as a bovine, equine, canine, ovine, or feline mammal, or a sheep, goat, llama, camel, or a rodent (rat, mouse), gerbil, or hamster. In a nonlimiting example, a subject is one who is infected with a pathogen, such as influenza virus, e.g., an H1, H5N1, H7, or H9 virus, or who is at risk of infection by such virus, or who is susceptible to such infection. In particular aspects as described herein, the subject is a human subject, such as a patient.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater, consecutively, such as to 100 or greater.

As used herein, the terms “treat,” treating,” “treatment,” and the like refer to reducing, diminishing, decreasing, abrogating, ameliorating, or eliminating, a disease, condition, disorder, or pathology, and/or symptoms associated therewith. While not intending to be limiting, “treating” typically relates to a therapeutic intervention that occurs after a disease, condition, disorder, or pathology, and/or symptoms associated therewith, have begun to develop so as to reduce the severity of the disease, etc., and the associated signs and symptoms. It will be appreciated that, although not precluded, treating a disorder or condition does not require that the disease, condition, disorder, pathology, or the symptoms associated therewith, be completely eliminated.

As used herein, the terms “prevent,” “preventing,” “prevention,” “prophylactic treatment” and the like, refer to inhibiting or blocking a disease state, or the full development of a disease in a subject, or reducing the probability of developing a disease, disorder or condition in a subject, who does not have, but is at risk of developing, or is susceptible to developing, a disease, disorder, or condition.

As referred to herein, a “transformed” cell is a cell into which a nucleic acid molecule or polynucleotide sequence has been introduced by molecular biology techniques. As used herein, the term “transformation” encompasses all techniques by which a nucleic acid molecule or polynucleotide may be introduced into such a cell, including transfection with viral vectors, transformation with plasmid vectors, and introduction of naked nucleic acid (DNA or RNA) by electroporation, lipofection, and particle gun acceleration.

By “vaccine” is meant a preparation of immunogenic material (e.g., protein or nucleic acid), such as a protein or peptide antigen, capable of stimulating (eliciting) an immune response, administered to a subject to treat a disease, condition, or pathology, or to prevent or protect against a disease, condition, or pathology, such as an infectious disease. The immunogenic material may include, for example, attenuated or killed microorganisms (such as attenuated viruses), or antigenic proteins, peptides or DNA derived from such microorganisms. Vaccines may elicit a prophylactic (preventative) immune response in the subject; they may also elicit a therapeutic response immune response in a subject. As mentioned above, methods of vaccine administration vary according to the vaccine, and can include routes or means, such as inoculation (intravenous or subcutaneous injection), ingestion, inhalation, or other forms of administration. Inoculations can be delivered by any of a number of routes, including parenteral, such as intravenous, subcutaneous or intramuscular. Vaccines may also be administered with an adjuvant to boost the immune response.

As used herein, a “vector” refers to a nucleic acid (polynucleotide) molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes in a host cell. In some embodiments of the present disclosure, the vector encodes an influenza HA, NA or M1 protein. In some embodiments, the vector is the pTR600 expression vector (U.S. Patent Application Publication No. 2002/0106798; Ross et al., 2000, Nat Immunol 1(2):102-103; and Green et al., 2001, Vaccine 20:242-248).

By “virus-like particle (VLP)” is meant virus particles made up of one of more viral structural proteins, but lacking the viral genome. Because VLPs lack a viral genome, they are non-infectious and yield safer and potentially more-economical vaccines and vaccine products. In addition, VLPs can often be produced by heterologous expression and can be easily purified. Most VLPs comprise at least a viral core protein that drives budding and release of particles from a host cell. One example of such a core protein is influenza M1. In some embodiments herein, an influenza VLP comprises the HA, NA and M1 proteins. As described herein, influenza VLPs can be produced by transfection of host cells with plasmids encoding the HA, NA and M1 proteins. After incubation of the transfected cells for an appropriate time to allow for protein expression (such as for approximately 72 hours), VLPs can be isolated from cell culture supernatants. By way of example, a protocol for purifying or isolating influenza VLPs from cell supernatants involves low speed centrifugation (to remove cell debris), vacuum filtration and ultracentrifugation of the VLPs through 20% glycerol. A virus-like particle may also include a subviral particle (SVP), which is typically smaller in size than a virus and constitutes a particle without a virus capsid or genome.

A “computer readable medium” refers to any article of manufacture that contains data that can be read by a computer (non-transitory media) or a carrier wave signal carrying data that can be read by a computer. Such computer readable media include, without limitation, magnetic media, such as a disk, tape, or cards; optical media such as CD-ROM or writeable compact disk; magneto-optical media in disk, tape, or card form; paper media, such as punched cards or paper tape; or on carrier wave signal received through a network, wireless network or modem, internet, including, but not limited to, radio-frequency signals, satellite signals, and infrared signals.

Unless specifically stated or obvious from context, as used herein, the term “or” is understood to be inclusive. Unless specifically stated or obvious from context, as used herein, the terms “a”, “an”, and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, the term “about” is understood as within a range of normal tolerance in the art, for example within 2 standard deviations of the mean. About may be understood as being within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 depicts a schematic flowchart of the steps of the method for generating a broadly reactive antigen for use in eliciting a broadly active immune response, e.g., in the form of neutralizing antibodies, as described herein. Sequences are first obtained from a group of viruses. The sequences are then aligned and partial sequences are removed. The remaining sequences are used to generate a phylogenetic tree, and clusters that are ≥95%-≥98% (particularly ≥98%) identical are extracted from the tree. These extracted sequences are then used to generate a “primary” (1°) ‘broadly reactive’ sequence with optimal alignment of identity among sequences. This process is repeated until all clusters from the phylogenetic tree have been addressed. Next all (1°) ‘broadly reactive’ sequences are consolidated and aligned with one another. A new phylogenetic tree is then generated using the 1° ‘broadly reactive’ sequences. From this tree clusters of ≥3 1° ‘broadly reactive’ sequences are extracted, and from these a “secondary” (2°) ‘broadly reactive’ sequence is generated. This process is repeated until all of the clusters of (1°) ‘broadly reactive’ sequences have been addressed. Using the “secondary” (2°) ‘broadly reactive’ sequences, a “backbone” ‘broadly reactive’ sequence is generated. The (2°) ‘broadly reactive’ sequences are then inserted into rolling scenarios 1, 2, and/or 3 (FIG. 2A) to generate final sequences. The final sequences are then checked for uniqueness against the sequences downloaded in step one.

FIGS. 2A-2D illustrate aspects of the methods described herein for generating non-naturally occurring, broadly reactive, pan-epitopic, influenza virus antigen sequences, taking into account influenza season and geographical location, e.g., 2002 Southern Hemisphere or 2002-2003 Northern Hemisphere influenza season. FIG. 2A presents three scenarios for rolling forward new antigen sequences into a backbone sequence. Following the design of the initial backbone sequence (described here as a set of sequences derived from Years 1-3), the three different scenarios shown depict how new sequences, from subsequent years, are added to the set of sequences derived from Years 1-3. FIGS. 2B-2D present further steps of the methods as described herein. FIG. 2B depicts steps in which primary sequences are first aligned by a season, and then are aligned as secondary sequences per season. All secondary alignments are compared and aligned into a multi-seasonal, secondary aligned sequence to generate the backbone sequence as referred to in FIG. 2A. FIG. 2C presents a schematic of the phylogenetic tree analysis associated with generating a broadly reactive, immunogenic HA sequence (BR HA sequence) in accordance with Scenario #1 of FIG. 2A. FIG. 2D presents a schematic of the phylogenetic tree analysis associated with generating sequences in accordance with Scenario #2 of FIG. 2A, in which involves extending a sequence by one season, and then another season.

FIG. 3 presents representative amino acid (aa) sequences of non-naturally occurring, broadly reactive, pan-epitopic HA antigens of H5 influenza virus generated using the methods as described herein and designated IAN-1 to IAN-8. Shown are the amino acid sequences of avian H5 HA proteins generated by the methods and the scenarios (1 and 3) of FIGS. 2A-2D. See, also, Examples 1 and 2. These HA proteins represent broadly reactive, amino acid sequences of complete H5 HA polypeptides. Typically, the leader sequence (at the N-terminus) comprises about 20 aa, as depicted in light gray shading in the Figure. The HA protein is composed of the HA1 (head portion), which follows the leader sequence, and the HA2 (tail or stalk portion) at the C-terminus, as depicted in darker gray shading, which follows the HA1 portion. When the IAN HA proteins were used as immunogens (or vaccines) to immunize animals, the antisera generated in response to immunization with these HA proteins was found inactivate HA from several different virus strains compared with controls. (See, FIGS. 7A-7H infra).

FIG. 4 provides a representative amino acid (aa) sequence of a broadly reactive neuraminidase (NA) N1 antigen of influenza virus produced by the described methods of generating non-naturally occurring, broadly reactive, pan-epitopic antigens.

FIG. 5 shows a graph demonstrating the results of virus challenge of mice that had been immunized with VLPs comprising influenza virus NA protein sequences derived from different virus types as shown, including a broadly reactive N1 immunogen generated according to the described methods. Balb/c mice (10 mice/group) received 3 intramuscular vaccinations of 3 μg neuraminidase (NA) VLP vaccines adjuvanted with an oil and water emulsion in 50 μL. Control mice received mock vaccination with PBS. Vaccine groups were as follows: (●): N1 from A/Chile/1/1983; (▪): N1 from A/Brisbane/59/2007; (▴): N1 from A/California/07/2009; (▾): mock (PBS); and (♦): Broadly reactive N1 antigen immunogen. Mice were challenged intranasally with 10⁶ PFU (plaque forming units) of A/California/07/2009 virus 8 weeks after the last vaccination. Percent of pre-challenged weight was observed. The animals that had been administered VLPs containing influenza virus N1 NA immunogen produced by the methods described herein showed significantly less weight loss at days 5-7 post challenge compared with the animals that had received VLPs containing NA sequences from the other influenza virus types and compared with control animals that had received PBS/adjuvant.

FIGS. 6A and 6B show the inhibition of neuraminidase (NA) activity by antibodies elicited against VLPs containing influenza N2 NA sequences as assessed using a neuraminidase inhibition assay. Balb/c mice (5 mice/group) received 3 intramuscular vaccinations of 3 μg NA VLP vaccines adjuvanted with an oil and water emulsion in 50 μL. Vaccine groups were as follows: (●): N2 from A/Panama/07/1999; (▪): N2 from A/Wisconsin/67/2005; (▴): N2 from A/Perth/16/2009; (▾): N2 from A/Texas/50/2012; and (♦): N2 from Switzerland/9715293/13. Sera were collected at day 42 post first vaccination, 2 weeks after the second vaccination and at day 70 post first vaccination, 2 weeks after the third vaccination. FIG. 6A shows 50% inhibition of N2 NA activity (neuraminidase inhibiting (NAI) antibody titer) against listed viruses at day 42. FIG. 6B shows 50% inhibition of N2 NA activity (neuraminidase inhibiting (NAI) antibody titer) against listed viruses at day 70.

FIGS. 7A-7H present graphs showing the results of hemagglutinin inhibition assays (HAI) performed using immunogens derived from avian hemagglutinin (HA) sequences and produced using the described methods. The immunogen was designed based on avian HA gene sequences that were submitted to the Global Initiative on Sharing All Influenza Data (GISAID) between years 2015-2018. Briefly, to obtain the results, an HA sequence (e.g., IAN-2 and each of IAN-4 through 8) was expressed as a soluble recombinant protein and was purified and formulated with an oil and water adjuvant (Addavax; InvivoGen, San Diego, Calif.). These protein immunogens were then used to vaccinate or immunize, i.e., intramuscularly administer to, Female Balb/C mice following a prime-boost-boost regimen (5 protein immunogen/mouse, at weeks 0, 4 and 8). Serum was collected from the immunized animals 2 weeks following the last boost (on week 10) and was treated with receptor-destroying enzyme (RDE) to inactivate nonspecific inhibitors and heat inactivated prior to performing hemagglutinin inhibition assays (HAI). H5Nx viruses containing PR8 internal genes, wild-type HA and neuraminidase (NA) were used to perform HAI assays using 1% horse blood. The PR8 viruses are a 6:2 reasortant with the 6 internal gene segments isolated from the A/Puerto Rico/8/1934 H1N1 virus and pseudotyped with an HA and NA protein. Wild-type HA and NA proteins are antigens isolated from human wild-type influenza viruses. Each set of mice was immunized with a single immunogen (vaccine) expressing one of the experimental HA antigens (FIGS. 7A-7F), a known immunogen (vaccine) (“Human COBRA-2”) used as a comparator control in the study (FIG. 7G), or an HA isolated from a wild-type H5N1 influenza virus (A/Whooperswan/Mongolia/244/2005), (FIG. 7H) which is classified as a clade 2.2 H5N1 virus, which served as a wild-type control for comparison of activity with the other immunogens tested. As observed in FIGS. 7A-7F, antisera generated against the ‘vaccine’ immunogens in the immunized animals showed a diverse response in inhibiting or neutralizing HA activity of a variety of PR8 H5Nx viruses in the HAI assay. By way of example, the IAN-8 immunogen showed high levels of inhibition against HA of five different H5 strains used in the assay (FIG. 7F). The virus strains used for the HAI panel were Si/14 (A/Sichuan/26221/2014), gy/Wa/14 (A/gyrfalcon/Washington/41088-6/2014), Hu/10 (A/Hubei/01/2010), Gu/13 (A/Guizhou/1/2013), WS/05 (A/whooper swan/Mongolia/244/2005).

FIGS. 8A and 8B show a phylogenetic tree and a table related to the phylogenetic diversity of neuraminidase (N1) derived from H1N1 and H5N1 influenza viruses compared with a neuraminidase polypeptide sequence generated by the described method herein (called “NextGen N1” in FIGS. 8A and 8B. Based on amino acid sequences, the NextGen N1 sequence was aligned with the N1 amino acid sequences of N1 derived from wild-type H1N1 and H5N1 influenza subtype sequences. In FIG. 8B, the percent matrix identity shows the percentage of amino acid residues that are identical between two neuraminidase polypeptide sequences. The darker background in the matrix indicates a higher percentage of identity between the sequences.

FIGS. 9A and 9B present graphs showing “percent of pre-challenged weight in vaccinated mice after challenge with influenza strains and percent animal survival as described in Example 8 herein. For the experiments, groups of 10 mice were immunized/“vaccinated” in a prime-boost-boost regimen with virus-like particles (VLP) expressing neuraminidase (NA) protein from wildtype virus strains (e.g., Chile/83, Brisbane/07, California/09), NextGen N1 NA produced by the methods described herein, or control (PBS/AFO3). Following boosting, the immunized/“vaccinated” animals were challenged with either H1N1 virus (A/California/07/2009 type) (FIG. 9A) or with H5N1 virus (A/Guizhou/1/2013 type) (FIG. 9B). The top graphs in FIGS. 9A and 9B show the percent of animals' pre-challenged weight after challenge with H1N1 (A/California/07/2009 type) virus or H5N1 virus A/Guizhou/1/2013, respectively. Weight and scores were recorded daily. The bottom graphs in FIGS. 9A and 9B show survival curves after challenge of vaccinated animals with H1N1 virus A/California/07/2009 (FIG. 9A) or with H5N1 virus A/Guizhou/1/2013 (FIG. 9B).

FIGS. 10A-10D present graphs showing inhibition activity of mouse serum antibodies using a Neuraminidase (NA) Enzyme Linked Lectin Assay (ELLA). Briefly, sera from mice immunized with NA immunogens from different virus types were collected 14 days after the first boost (d42) and tested against a panel of H1N1 and H5N1 viruses (see FIGS. 9A and 9B) using ELLA. In the graphs, the dotted line represents 50% inhibition of NA activity. As seen in the graphs, the sera groups tested were as follows: (●, “Chile sera”) sera from animals immunized with NA immunogen derived from A/Chile/1/1983 virus strain; (▪, “Bris sera”) sera from animals immunized with NA immunogen derived from A/Brisbane/59/2007 virus strain; (▴, “Cal sera”) sera from animals immunized with NA immunogen derived from A/California/07/2009 virus strain; (▾, “NextGen N1 Sera”) sera from animals immunized with N1 pan-epitopic immunogen and (♦) sera from animals receiving PBS (negative control).

ELLAs were performed as described (Couzens et al., 2014. J. Virol. Methods, Vol. 210, pp. 7-14). Briefly, flat-bottom nonsterile 96-well tissue culture plates (Maxisorp, Nunc) were coated with 100 μL of fetuin (Sigma) at 25 μg/ml at 4° C. overnight. 50 μL of antibodies were serially diluted two-fold in Dulbecco's phosphate buffered saline (DPBS) with 0.05% Tween 20 and 1% BSA (DPBST-BSA) and then incubated in duplicate fetuin-coated plates with an equal volume of the selected antigen diluted in DPBST-BSA. These plates were subsequently sealed and incubated for 18 hours at 37° C. The plates were subsequently washed three times with PBS/0.05% Tween 20, and 100 μL/well of HRP-conjugated peanut agglutinin lectin (PNA-HRPO, Sigma-Aldrich) in DPBST-BSA were added to each well. The plates were incubated for 2 hours at room temperature in the dark; washed three times; and developed with OPD (Sigma). Absorbance was read at 492 nm on a microplate spectrophotometer (BioTek). Data points were analyzed using Prism software, and the 50% inhibition concentration (IC₅₀ value) was defined as the concentration at which 50% of the NA activity was inhibited compared to the negative control.

FIG. 11 shows the amino acid sequence of the H1 V9 hemagglutinin (HA) antigen.

FIGS. 12A-12D show graphs of hemagglutinin inhibition following immunization of mice with VLPs expressing HA antigen from the source shown in the figures. BALB/c mice (8 animals/immunogen) were immunized with VLPs expressing the HA antigens as shown above each graph (FIG. 12A: Mock control; FIG. 12B: HA antigen derived from Bris/07 virus strain; FIG. 12C: HA antigen derived from CA/09 virus strain; and FIG. 12D: HA antigen H1 V9 as shown in FIG. 11). The sera from each mouse were collected and assessed for HA neutralization/inhibition in an HAI assay as described in Example 3. using the virus strain panel shown on the x-axis. The horizontal gray bar indicates an antiserum titer of 1:40-1:80. A titer of 1:40 is considered to provide the minimal protection in humans for seasonal influenza. Overall, it was observed that the V9 HA antigen used as immunogen elicited sera in immunized animals that had broader HAI activity against the panel of H1N1 viruses compared with sera from animals immunized with HA from wild-type virus strains, i.e., Bris/07 (FIG. 12B) and CA/09 (FIG. 12C).

FIG. 13 presents the amino acid sequences of H2N1 HA protein antigens, designated as Z1-Z9, produced by the methods as described herein, which generate broadly reactive antiserum antibodies in animals when used as immunogens. The H2N1 HA immunogen Z1 was designed by the described methods and was derived from all H2 HA sequences, i.e., from human, avian and mammalian influenza strains, from 1957 to the present. The H2N1 HA immunogen Z2 was designed by the described methods and was derived from Clade 2 H2 HA sequences from avian and mammalian influenza strains, from the 1980s to the present. The H2N1 HA immunogen Z3 was designed by the described methods and was derived from Clade 1 H2 HA sequences from avian and mammalian influenza strains, from the 1980s to the present. The H2N1 HA immunogen Z4 was designed by the described methods and was derived from Clade 1 H2 HA sequences from avian and mammalian influenza strains, from the 1980s to the present. The H2N1 HA immunogen Z5 was designed by the described methods and was derived from Clade 2 H2 HA sequences from human and avian influenza strains, from 1957 to the 1970s. The H2N1 HA immunogen Z6 was designed by the described methods and was derived from Clade 2 H2 HA sequences from human and avian influenza strains, from 1957 to the 1970s. The H2N1 HA immunogen Z7 was designed by the described methods and was derived from Clade 3 H2 HA sequences from avian and mammalian influenza strains, from the 1970s to the 2000s. The H2N1 HA immunogen Z8 was designed by the described methods and was derived from Clade 3 H2 HA sequences from avian and mammalian influenza strains, from the 1970s to the 2000s. The H2N1 HA immunogen Z9 was designed by the described methods and was derived from Clade 3 H2 HA sequences from avian and mammalian influenza strains, from the 1970s to the 2000s.

FIGS. 14A-14D show graphs of hemagglutinin inhibition activity of antibodies in the antisera of mice following immunization with VLPs expressing each of the H2N1 HA antigens Z1-Z7 as described above and shown in FIG. 13. BALB/c mice (8 mice per immunogen tested) were immunized with VLPs expressing the H2N1 HA antigens Z1-Z7, as shown above each graph. The sera from each mouse were collected and assessed in an HAI assay. All of the viruses tested against the sera in the HAI assay are presented on the x-axes. The horizontal gray bar indicates an antiserum titer of 1:40-1:80. A titer of 1:40 is considered to provide the minimal protection in humans for seasonal influenza. Overall, the HAI assay results indicate that, following immunization, the Z1, Z3, Z5, and Z7 H2 HA protein immunogens generated broadly reactive antisera that neutralized HA from a variety of virus strains as well as, or better than, antisera from animals immunized with control wild-type virus (Mallard/Netherlands/2001), which typically produces high titer antisera in animals following immunization.

### DETAILED DESCRIPTION OF THE DISCLOSURE

Featured herein are methods for generating synthetic (non-naturally occurring) broadly reactive antigens and antigen sequences, e.g., protein and glycoprotein antigens, derived from pathogenic organisms (microorganisms), which can elicit a potent immune response in a subject, particularly, a human subject. Such immunogenic antigens are also referred to as “immunogens,” “vaccine immunogens,” or “vaccines” herein.

In one embodiment, the methods involve the generation of non-naturally occurring, broadly reactive, pan-epitopic immunogens, including vaccines, based on the influenza (“flu”) virus hemagglutinin (HA) protein or neuraminidase (NA) that elicit a broadly active immune response, in some cases, against seasonal pathogen strains, such as influenza strains (and drift variants) spanning several years, including drifted strains not yet in existence. The elicitation of such a broadly active immune response serves to treat infection and disease caused by the pathogen (e.g., flu caused by influenza virus) and the symptoms thereof.

In another embodiment, the methods are based on the surface proteins or glycoprotein antigens of other pathogenic organisms, for example, pathogenic microorganisms such as non-influenza viruses, bacteria, fungi, protozoa, parasites and the like, and generate non-naturally occurring, broadly reactive, immunogenic antigens, including vaccines, that elicit a broadly active immune response against the pathogenic organisms (microorganisms) that express the antigens, particularly, antigens expressed on a pathogen's surfaces. In an embodiment, the method is useful for generating broadly reactive immunogenic antigens from other viruses, such as, without limitation, Dengue, Foot and Mouth Disease, Chikungunya virus, Zika and Rift Valley Fever.

Also featured herein are the fully synthetic and immunogens, such as pathogen-derived antigens and viral HA or NA antigens, generated by the methods. Such antigens, e.g., HA or NA protein antigens and other pathogenic protein antigens, are synthetic proteins not found in nature (non-naturally occurring), yet they retain all of the functions of a natural pathogen protein, e.g., the HA or NA viral protein, or the pathogenic protein, and are immunogenic, i.e., they can elicit an immune response, in particular, a broadly active immune response in the form of neutralizing antibodies and/or reactive T lymphocytes, following administration or delivery to, or introduction into, a subject. Also provided are immunogenic compositions, e.g., vaccines, comprising the synthetic, pathogen-derived immunogenic antigens or virus antigens, or nucleic acids encoding the antigens.

In an embodiment and in accordance with the approaches and methods described herein, an influenza virus HA or NA amino acid sequence and a protein antigen having such sequence are generated for use as an immunogen, immunogenic composition, e.g., a vaccine, that elicits a broadly reactive immune response in a subject, particularly a human subject, to whom the composition is administered. The immunogen or vaccine is generated from the focused, comparative analysis and clustering of many HA or NA amino acid sequences of a flu virus strain so as to afford broadly active protection against flu virus proteins having sequence variability and similarity over several years. The resulting virus immunogens comprise antigenic determinants that represent different “antigenic spaces” derived from the sequences of many virus strains analyzed based on seasonal periods of time (either overlapping or non-overlapping seasonal time periods).

The assessed overlapping or non-overlapping seasonal time periods may encompass different intervals of time, for example, 5 months, 6 months, 7 months, eight months, nine months, 10 months, 11 months, 1 year, 2 years, 3 years, 4 years, 5 years, 6 years, 7 years, or 8 years, including time intervals therebetween. The overall time period over which the analysis of HA or NA sequence identities and differences are made, based on the HA or NA sequences assessed in the more defined time periods, may also encompass different lengths of time, such as an overall time period of about or equal to 10 years, about or equal to 15 years, about or equal to 20 years, about or equal to 25 years, about or equal to 30 years, about or equal to 40 years, about or equal to 50 years, and the like, including time intervals therebetween.

The methods as described herein involve the generation of seasonal pan-epitopic, broadly reactive antigens of pathogens, e.g., viruses, especially antigens such as virus HA and NA antigens containing sequences based on drift variants, wherein the antigens are designed to generate a broadly active immune response, particularly in the form of neutralizing antibodies, in a subject, particularly a human subject. As described infra, Examples 1 and 2 relate to the design of a method to generate and update on a seasonal/annual basis immunogenic antigens derived from pathogens (e.g., viruses, bacteria, etc.), wherein the antigens have broadly reactive antigenic determinants. Such a design is beneficial for eliciting an immune response (e.g., production of neutralizing antibodies) against the pathogens where multiple strains of the pathogen co-circulate at one time. The method further provides immunogenic antigens derived from pathogens, particularly, viruses that frequently mutate parts of their genomes to escape immune pressure, and as a consequence, evade immune surveillance in a subject whose immune system is not primed or stimulated to generate antibodies against antigenic epitopes (determinants) on the pathogenic antigens following infection. The methodology described herein results in the design of synthetic antigens having amino acid (or polynucleotide) sequences that will elicit greater numbers of neutralizing antibodies against drift variants within and across multiple seasons compared with wild-type antigen sequences.

In an embodiment, the broadly reactive, synthetic influenza proteins, immunogens and vaccines described herein are advantageous in that they are designed to provide broader and longer-lasting protection against several seasonal flu strains (or clades) prevalent in different geographical locations. In addition, because the described methods do not rely on the annual selection of a given strain for making an immunogen or vaccine, the year-round manufacturing of a flu vaccine that is broadly cross-protective against many strains or clades of flu viruses is achieved. Thus, the methods described herein afford a universal and broad-spectrum flu vaccine that may alleviate the need for a seasonal flu vaccine against virus strains and subtypes of influenza virus that is administered annually.

In particular embodiments, the immunogenic influenza virus HA (or NA) antigens generated from the methods described herein may be used in immunogenic compositions (e.g., influenza vaccines) that are capable of affording protective immunity against influenza infection and disease in a subject. The protective immunity is provided in the subject through the elicitation of potent, broadly reactive, anti-HA (or NA) specific antibody responses that protect the subject against drifted, seasonal influenza virus strains and pandemic influenza virus strains. The methods provide an advantage over prior and traditional processes of manufacturing immunogenic compositions directed against influenza virus strains and subtypes (e.g., influenza vaccines), which typically depend on the selection of candidate vaccine viruses by public health authorities following analysis of data collected through active surveillance of influenza viruses circulating each year.

### Influenza Virus

Influenza viruses are segmented negative-strand RNA viruses that belong to the Orthomyxoviridae family. There are three types of Influenza viruses: types A, B and C. Influenza A viruses infect a wide variety of birds and mammals, including humans, horses, marine mammals, pigs, ferrets, and chickens. In animals, most influenza A viruses cause mild localized infections of the respiratory and intestinal tract. However, highly pathogenic influenza A strains, such as, for example, the H1N1 (“H1”) or H5N1 (“H5”), or H7 or H9 strains, cause systemic infections in poultry in which mortality may reach 100%. Animals infected with influenza A often act as a reservoir for the influenza viruses and certain subtypes have been shown to cross the species barrier to humans in whom they can cause severe disease and devastating flu outbreaks that can lead to death of the infected human subjects.

Influenza A viruses can be classified into subtypes based on allelic variations in antigenic regions of two genes that encode surface glycoproteins, namely, hemagglutinin (HA) and neuraminidase (NA) which are required for viral attachment and cellular release, respectively. Currently, sixteen subtypes of HA (H1-H16) and nine NA (N1-N9) antigenic variants are known for influenza A virus. Previously, only three subtypes were known to circulate in humans (H1N1 or H1N2). However, in recent years, for example, the pathogenic H5N1 subtype of avian influenza A has been reported to cross the species barrier and infect humans as documented in Hong Kong in 1997 and 2003, leading to the death of several patients.

In humans, the avian influenza virus infects cells of the respiratory tract as well as the intestinal tract, liver, spleen, kidneys and other organs. Symptoms of avian influenza infection include fever, respiratory difficulties, including shortness of breath and cough, lymphopenia, diarrhea and difficulties regulating blood sugar levels. In contrast to seasonal influenza, the group most at risk is healthy adults which make up the bulk of the population. Due to the high pathogenicity of certain avian influenza A subtypes, particularly H5N1, and their demonstrated ability to cross over to infect humans, there is a significant economic and public health risk associated with these viral strains, including a real epidemic and pandemic threat. Currently, no effective vaccines for H5N1 infection are available.

The influenza A virus genome encodes nine structural proteins and one nonstructural (NS1) protein with regulatory functions. The influenza virus segmented genome contains eight negative-sense RNA (nsRNA) gene segments (PB2, PB1, PA, NP, M, NS, HA and NA) that encode at least ten polypeptides, including RNA-directed RNA polymerase proteins (PB2, PB 1 and PA), nucleoprotein (NP), neuraminidase (NA), hemagglutinin, e.g., subunits HA1, frequently referred to as the “head” subunit; and HA2, frequently referred to as the “tail” or “stalk” subunit; the matrix proteins (M1 and M2); and the non-structural proteins (NS1 and NS2) (See, e.g., Krug et al., 1989, In: The Influenza Viruses, R. M. Krug, ed., Plenum Press, N.Y., pp. 89 152).

The ability of influenza virus to cause widespread disease is due to its ability to evade the immune system by undergoing antigenic change, which is believed to occur when a host is infected simultaneously with both an animal influenza virus and a human influenza virus. During mutation and reassortment in the host, the virus may incorporate an HA and/or NA surface protein gene from another virus into its genome, thereby producing a new influenza subtype and evading the immune system.

Because of antigenic variation (drift) in the circulating strains of influenza viruses, in particular, in the HA and NA proteins of the virus, the efficacy of immunogenic compositions, e.g., vaccines, against influenza virus has frequently been less than optimal and sub-par. The methods described herein provide broadly reactive, pan-epitopic HA or NA antigens that generate a broadly reactive immune response, particularly, in the form of neutralizing antibodies that bind to the viral antigens and neutralize the activity of the virus (e.g., its ability to infect cells), to treat influenza and its symptoms more effectively.

### Influenza Virus Hemagglutinin (HA) and Neuraminidase (NA) Proteins

HA is a viral surface glycoprotein that generally comprises approximately 560 amino acids (e.g., 566 amino acids) and represents 25% of the total virus protein. As described herein, HA is a protein antigen that is highly useful as an immunogen because it contains a diverse repertoire of epitopes against which antibodies are generated in a subject or host that encounters the HA antigen of influenza viruses during infection.

HA is responsible for adhesion of the viral particle to, and its penetration into, a host cell, particularly, in the respiratory epithelium, in the early stages of infection. Cleavage of the virus HA0 precursor into the HAI and HA2 sub-fragments is a necessary step in order for the virus to infect a cell. Thus, cleavage is required in order to convert new virus particles in a host cell into virions capable of infecting new cells. Cleavage is known to occur during transport of the integral HA0 membrane protein from the endoplasmic reticulum of the infected cell to the plasma membrane. In the course of transport, HA undergoes a series of co- and post-translational modifications, including proteolytic cleavage of the precursor HA into the amino-terminal fragment HAI (“head”) and the carboxy terminal HA2 (“tail” or “stalk”). One of the primary difficulties in growing influenza strains in primary tissue culture or established cell lines arises from the requirement for proteolytic cleavage activation of the influenza hemagglutinin in the host cell.

Although it is known that an uncleaved HA can mediate attachment of the virus to its neuraminic acid-containing receptors on a cell surface, it is not capable of the next step in the infectious cycle, which is fusion. It has been reported that exposure of the hydrophobic amino terminus of HA2 by cleavage is required so that it can be inserted into the target cell, thereby forming a bridge between the virus and the target cell membranes. This process is followed by fusion of the two membranes and entry of the virus into the target cell.

Proteolytic activation of HA involves cleavage at an arginine residue by a trypsin-like endoprotease, which is often an intracellular enzyme that is calcium-dependent and has a neutral pH optimum. Since the activating proteases are cellular enzymes, the infected cell type determines whether the HA is cleaved. The HA of the mammalian influenza viruses and the nonpathogenic avian influenza viruses are susceptible to proteolytic cleavage only in a restricted number of cell types. On the other hand, HA of pathogenic avian viruses among the H5 and H7 subtypes are cleaved by proteases present in a broad range of different host cells. Thus, there are differences in host range resulting from differences in hemagglutinin cleavability which are correlated with the pathogenic properties of the virus.

Neuraminidase (NA) is a second membrane glycoprotein of the influenza viruses. The presence of viral NA has been shown to be important for generating a multi-faceted protective immune response against an infecting virus. For most influenza A viruses, NA is 413 amino acids in length and is encoded by a gene of 1413 nucleotides. Nine different NA subtypes have been identified in influenza viruses (N1, N2, N3, N4, N5, N6, N7, N8 and N9), all of which have been found among wild birds. NA is involved in the destruction of the cellular receptor for the viral HA by cleaving terminal neuraminic acid (also called sialic acid) residues from carbohydrate moieties on the surfaces of infected cells. NA also cleaves sialic acid residues from viral proteins, preventing aggregation of viruses. Using this mechanism, it is hypothesized that NA facilitates the release of viral progeny by preventing newly formed viral particles from accumulating along the cell membrane, as well as by promoting transportation of the virus through the mucus present on the mucosal surface. NA is an important antigenic determinant that is subject to antigenic variation.

In addition to the surface proteins HA and NA, influenza virus comprises six additional internal genes, which give rise to eight different proteins, including polymerase genes PB1, PB2 and PA, matrix proteins M1 and M2, nucleoprotein (NP), and non-structural proteins NS1 and NS2 (See, e.g., Horimoto et al., 2001, Clin Microbiol Rev. 14(1):129-149).

For packaging into progeny virions, viral RNA is transported from the nucleus as a ribonucleoprotein (RNP) complex composed of the three influenza virus polymerase proteins, the nucleoprotein (NP), and the viral RNA, in association with the influenza virus matrix 1 (M1) protein and nuclear export protein (Marsh et al., 2008, J Virol, 82:2295-2304). The M1 protein that lies within the envelope is thought to function in assembly and budding. A limited number of M2 proteins are integrated into the virions (Zebedee, 1988, J. Virol. 62:2762-2772). These M2 proteins form tetramers having H+ ion channel activity, and when activated by the low pH in endosomes, acidify the inside of the virion, thus facilitating its uncoating (Pinto et al., 1992, Cell 69:517-528). Amantadine is an anti-influenza drug that prevents viral infection by interfering with M2 ion channel activity, thus inhibiting virus uncoating.

NS1, a nonstructural protein, has multiple functions, including regulation of splicing and nuclear export of cellular mRNAs as well as stimulation of translation. The major function of NS1 seems to be to counteract the interferon activity of the host, since an NS1 knockout virus was viable, although it grew less efficiently than the parent virus in interferon-nondefective cells (Garcia-Sastre, 1998, Virology 252:324-330).

The NS2 nonstructural protein has been detected in virus particles (Richardson et al., 1991, Arch. Virol. 116:69-80; Yasuda et al., 1993, Virology 196:249-255). The average number of NS2 proteins in a virus particle was estimated to be 130-200 molecules. An in vitro binding assay has demonstrated direct protein-protein contact between M1 and NS2. NS2-M1 complexes have also been detected by immunoprecipitation in virus-infected cell lysates. The NS2 protein is thought to play a role in the export of the RNP from the nucleus through interaction with M1 protein (Ward et al., 1995, Arch. Virol. 140:2067-2073).

### Influenza Proteins and Virus-Like Particles (VLPs)

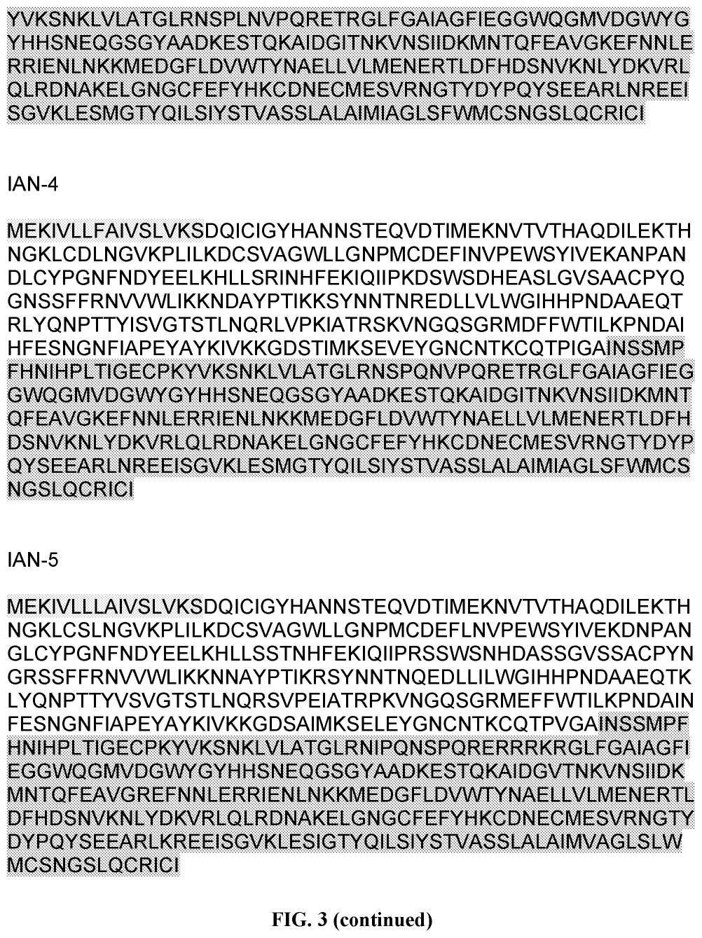

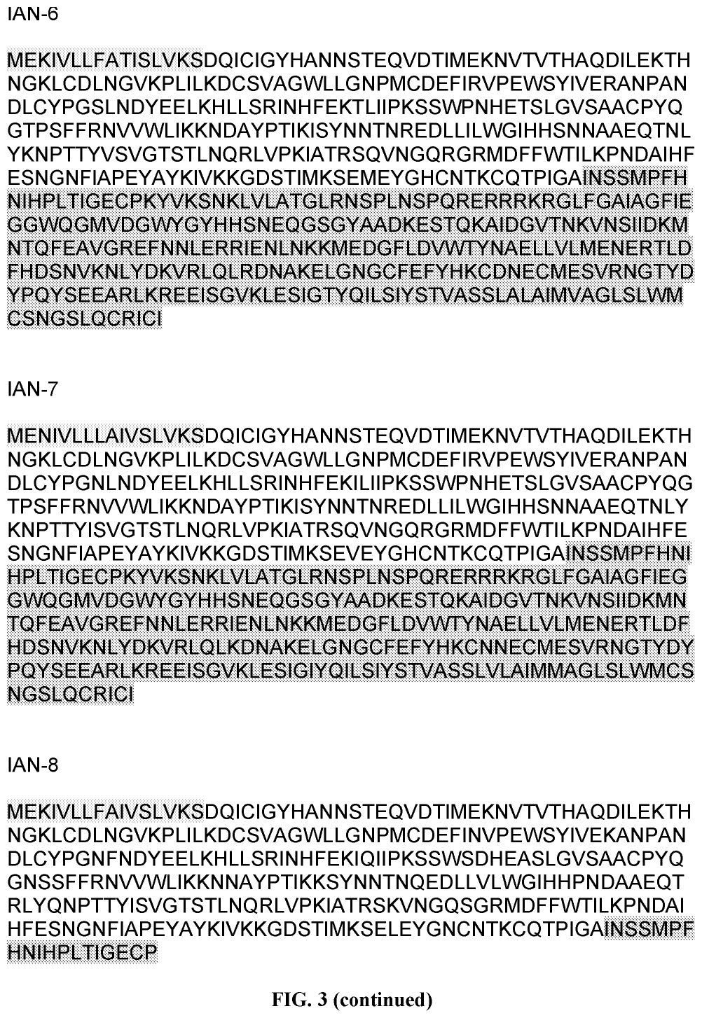

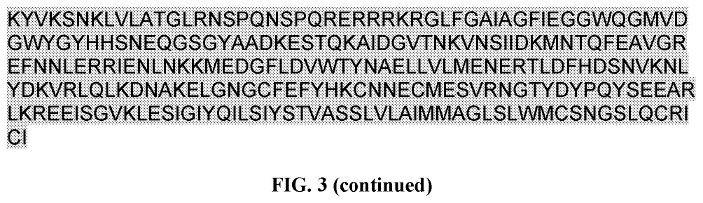

Provided by the described methods are non-naturally occurring, broadly reactive, pan-epitopic influenza polypeptides (immunogens) and influenza virus-like particles (VLPs) comprising an influenza immunogen containing diverse epitopes (antigenic determinants) that endow the antigen with the ability to generate a broadly active immune response against influenza and its symptoms, either prophylactic or therapeutic, following administration and delivery to a susceptible subject. By way of example, representative HA antigen sequences generated by the practice of methods described herein are presented in FIG. 3. In particular examples, the broadly reactive, pan-epitopic HA and/or NA polypeptides are administered as part of a VLP.

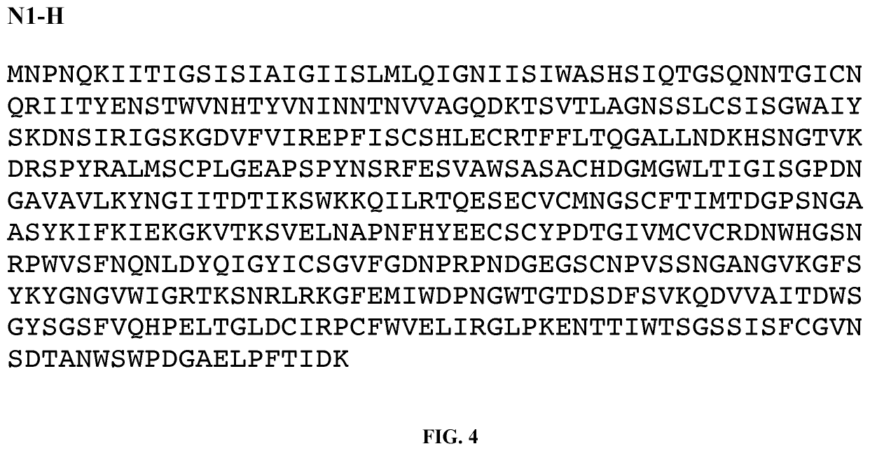

By way of further example, representative NA immunogenic antigen sequences generated by the practice of methods described herein are presented in FIG. 4.

It will be understood that the influenza virus immunogens and sequences described and provided herein are non-naturally occurring, broadly reactive and pan-epitopic, whether or not these characteristics and features are explicitly stated. It will be further understood that the antigen proteins generated by the methods described herein and used as immunogens are non-naturally occurring or synthetic antigens that elicit an immune response, e.g., neutralizing antibodies, in a subject.

The influenza VLPs include the viral HA, NA and M1 proteins. The production of influenza VLPs has been described in the art and is within the skill and expertise of one of ordinary skill in the art. Briefly, and as described, influenza VLPs can be produced by transfection of host cells with one or more plasmids containing polynucleotide sequences that encode the HA, NA and M1 proteins. After incubation of the transfected cells for an appropriate time to allow for protein expression (such as for approximately 72 hours), VLPs can be isolated from cell culture supernatants. Influenza VLPs can be purified from cell supernatants using procedures practiced in the art, for example, VLPs can isolated by low speed centrifugation (to remove cell debris), vacuum filtration and ultracentrifugation through 20% glycerol. In an embodiment, VLPs containing broadly reactive antigens derived from other pathogens can also be produced, isolated and used as immunogens or in immunogenic compositions.

The influenza VLPs can be used as influenza vaccines to elicit an immune response against H5N1 influenza viruses. In particular, the component, broadly reactive, pan-epitopic influenza HA polypeptides of the vaccines (or VLPs) contain antigenic (pan-epitopic) determinants that are broadly reactive and serve to elicit an immune response in a subject (e.g., the production of neutralizing antibodies and/or activated T-cells) that can treat a virus-infected subject (e.g., neutralize the infecting virus) and/or protect a subject against full-blown virus infection or the signs and symptoms thereof.

Methods of Generating Non-Naturally Occurring, Pathogen-Derived, Immunogenic Antigens which Elicit a Broadly Active Immune Response in a Subject

Described herein are methods for generating a non-naturally occurring, broadly reactive and immunogenic antigen of a pathogen, such as a virus or bacterium, in which the sequence of the antigen contains a diverse repertoire of epitopic determinants that can reflect antigenic drift and sequence variability in the pathogens' antigenic proteins, for example, over seasons (time) and in different geographic locations. In particular, the methods described herein are used to generate an antigen, such as the HA or NA viral antigens, having an amino acid sequence that contains antigenic determinants (epitopes) derived from sequence diverse virus strains, including drift variants, against which broadly reactive neutralizing antibodies can be raised, especially when the antigen is used as an immunogenic product, (an immunogen), e.g., an antiviral vaccine, that is introduced into a subject. The methods described herein take into account both antigen sequence identities and variabilities in clustering sequences to produce secondary and tertiary sequences, as well as build on sequence identity and diversity over seasons and geography to improve immunogenicity, in generating broadly reactive and pan-epitopic immunogens, thus providing a next stage technology relative to the approach described in U.S. Pat. Nos. 8,883,171, 9,212,207, 9,309,290, 9,555,095, 9,566,327, 9,566,328 and 9,580,475.

The methods described herein provide a next generation technology for generating non-naturally occurring, immunogen sequences that can be derived from a variety of pathogens, for example, virus and bacterial pathogens. For influenza virus, relevant antigens for providing a highly immunogenic sequence include, without limitation, the surface proteins hemagglutinin (HA), which is responsible for binding and entry into host epithelial cells, and neuraminidase (NA), which is involved in the process of budding new virions from host cells. The antigenic determinants or sites recognized on the hemagglutinin and neuraminidase proteins by host immune systems are under constant selective pressure. Antigenic drift allows for evasion of these host immune systems by small mutations in the hemagglutinin and neuraminidase genes that make the proteins unrecognizable to pre-existing host immunity. In particular, antigenic drift and the resulting drift variants involve a continuous process of genetic and antigenic change among flu strains. Illustratively and without limitation, the method is useful for generating immunogens, such as HA and NA proteins, from various influenza virus strains, such as HA derived from H1, H5, H7 and H9 influenza strains. The method is also useful for generating immunogens from other virus types, such as Dengue virus, Foot and Mouth Disease virus, Chikungunya virus, Zika and Rift Valley Fever virus.

The methods described herein include the evaluation of relevant parameters for analyzing and generating a composite viral antigen sequence that comprises epitopes reflecting sequence variability among many viruses, and involve a more comprehensive analysis and repeated clustering of different populations of antigen sequences of viruses present in different seasons or of different virus clades or types. The methods described herein achieve a composite viral antigen amino acid sequence that includes epitopic determinants ultimately derivable from both past and more recent seasons of virus infection or disease, and/or from viruses in different geographical locales, and/or from viruses in different clades or types, i.e., a “pan-epitopic” antigen that elicits a broadly reactive immune response when used as an immunogen.

The present methods provide the generation of a non-naturally occurring and pan-epitopic viral antigen that can elicit a broad immunogenic response when used as an immunogen, such as a vaccine (or as a component of a virus-like particle), and can involve the assessment of one or more of the following parameters: seasonality, (i.e., sequences of viral HA (or NA) antigens from past seasons and more recent seasons are assessed); geographical location, (i.e., sequences of viral HA (or NA) antigens from viruses in different geographic areas or regions of the world where virus outbreaks and infection have occurred are assessed); or different clades or subtypes (i.e., sequences of viral HA (or NA) antigens derived from viruses of different clades, types, or subtypes are assessed).

In general, the methods described herein provide a non-naturally occurring, broadly reactive, pan-epitopic pathogen-derived antigen sequence that is enhanced in the repertoire of epitopes that it contains. By way of example, a viral HA (or NA) protein antigen generated by the methods is ultimately derived from a vast number of sequences that are clustered and ordered based on their sequence identity, degree of genetic relatedness and degree of genetic variability, so as to arrive at a final protein sequence, following successive (iterative) rounds of sequence clustering and sequence identity analysis, that includes a greater number of diverse antigenic determinants or epitopes per single protein antigen generated as a result of the methodology, for example, when compared with previous approaches or techniques. The antigen sequences of immunogens generated by the methods can encompass epitopes that result from antigenic changes in the sequences of surface antigens of pathogens, such as influenza viruses, for example, that arise from point mutations during viral replication, giving rise to new influenza variants. As a result, the administration to a subject of an immunogen generated by the methods described herein can elicit an immune response in the subject that is directed against epitopes reflecting such antigenic changes.

A broadly reactive HA (or NA) antigen obtained by the described methods and used as an immunogen or immunogenic composition, such as a vaccine, elicits a broadly reactive immune response in an immunocompetent subject. Thus, the present methodology of generating a pan-epitopic and immunogenic protein antigen (e.g., influenza HA protein) provides a superior vaccine that captures the antigenic epitopes of many different influenza isolates, against which broadly active immune responses (e.g., broadly active neutralizing antibodies) are generated. It is noted that the terms “broadly active” and “broadly reactive” are used synonymously herein.

In an embodiment, a method of generating an antigen (e.g., a glycoprotein antigen) of a disease-causing pathogen, such as a virus, e.g., influenza virus is provided, in which the amino acid sequence of the antigen comprises a composite sequence representing a high proportion of the major and minor epitopes/antigenic determinants derived from the assessment of many amino acid sequences of the antigen by clustering the sequences based on sequence identities and differences, and selecting the most representative antigen sequences of the pathogen, taking into account different time periods (seasons in which disease or infection caused by the pathogen are prevalent), e.g., linear time periods; geographic areas (where disease or infection caused by the pathogen are prevalent), and/or clades, types, or subtypes of the pathogen. Such a pan-epitopic antigen serves as a potent immunogen and comprises a vaccine that is recognized and bound by broadly active neutralizing antibodies in a subject exposed to the pathogen.

Methods of generating a pathogen-derived, antigen sequence and expressed immunogenic antigen (e.g., a polypeptide, peptide, or a polynucleotide) are described herein. The pathogen-derived antigen generated by the methods elicits an immune response after introduction into a subject, in particular, an immune response in which broadly reactive neutralizing antibodies directed against the antigen are produced. In an embodiment, the antigen is a polypeptide or peptide antigen of a pathogen which currently causes disease or infection and its symptoms, such as a seasonal pathogen (e.g., influenza) or a pathogen native to certain geographical locales. In another embodiment, the antigen is a polypeptide or peptide antigen of a pathogen which will, in future, cause disease and its symptoms, such as a seasonal pathogen (e.g., influenza) or a pathogen native to certain geographical locales. In an embodiment, the pathogen-derived antigen is a polynucleotide sequence. In certain embodiments, the pathogen is a virus, in particular, influenza virus, and the antigen is the virus HA or NA protein. By way of example, representative HA and NA broadly reactive antigens generated by the methods described herein are shown in FIGS. 3 and 4.

As described infra, the methods, particularly those pertaining to the generation of antigens from virus sequences, may further involve a consideration of certain parameters, such as clade, species, season, or geography, depending on the virus type or strain, as described herein.

In one embodiment, a method of generating a non-naturally occurring, broadly reactive, pan-epitopic immunogen capable of generating an immune response in a subject is provided, in which the method comprises (a) generating a phylogenetic tree comprising full length related antigen sequences derived from one or more pathogen strains; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; and (f) generating a phylogenetic tree comprising the secondary sequences, wherein branches of the tree are combined if the substitution rate per amino acid site distance is less than about 0.001, thereby generating a non-naturally occurring immunogen comprising a pan-epitopic immunogen sequence. In an embodiment of the method, the pathogen is a virus, bacterium, fungus, protozoan, prion, and the like. In an embodiment, the bacterium is a Gram positive or Gram negative bacterium. In another embodiment of the method, the pathogen is a retrovirus or a DNA virus. In embodiments, the virus is Chikungunya, Dengue, Foot and Mouth disease, Influenza virus, Zika, or Rift Valley Fever Virus. In a particular embodiment, the virus is influenza (flu) virus, which may be present in the Northern or Southern hemispheres. In an embodiment, each cluster of antigen sequences in step (b) has at least 95%-98% sequence identity. In an embodiment, each cluster of antigen sequences in step (b) has at equal to or greater than 98% sequence identity.

In another embodiment, a method of generating a non-naturally occurring, broadly reactive, pan-epitopic immunogen capable of generating an immune response against present and future pathogens in a subject is provided, in which the method comprises (a) generating a phylogenetic tree comprising full length related antigen sequences derived from one or more pathogen strains, wherein each pathogenic strain present in two or more selected geographic regions over one or more selected periods of time; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; and (f) generating a phylogenetic tree comprising the secondary sequences, wherein branches of the tree are combined if the substitution rate per amino acid site distance is less than about 0.001, thereby generating a non-naturally occurring immunogen comprising a pan-epitopic immunogen sequence. In an embodiment of the method, the pathogen is a virus, bacterium, fungus, protozoan, prion, and the like. In an embodiment, the bacteria are Gram positive or Gram negative. In another embodiment of the method, the pathogen is a retrovirus or a DNA virus. In embodiments, the virus is Chikungunya, Dengue, Foot and Mouth disease, Influenza virus, Zika, or Rift Valley Fever Virus. In a particular embodiment, the virus is Influenza, which may be present in the Northern or Southern hemispheres. In an embodiment, each cluster of antigen sequences in step (b) has at least 95%-98% sequence identity. In an embodiment, each cluster of antigen sequences in step (b) has at equal to or greater than 98% sequence identity.

In another embodiment, a method of generating a non-naturally occurring, broadly reactive, pan-epitopic immunogen capable of generating an immune response against present and future influenza virus strains in a subject is provided, in which the method comprises (a) generating a phylogenetic tree comprising full length related antigen sequences derived from one or more influenza virus strains, wherein each influenza virus strain is present in two or more selected geographic regions over one or more flu seasons; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; and (f) generating a phylogenetic tree comprising the secondary sequences, wherein branches of the tree are combined if the substitution rate per amino acid site distance is less than about 0.001, thereby generating a non-naturally occurring immunogen comprising a pan-epitopic immunogen sequence. In an embodiment, each cluster of antigen sequences in step (b) has at least 95%-98% sequence identity. In an embodiment, each cluster of antigen sequences in step (b) has at equal to or greater than 98% sequence identity.

In another embodiment, a method of obtaining a broadly reactive and immunogenic HA or NA antigen of an influenza H1 virus and viruses related thereto is provided. In the practice of the method, the parameters of HA or NA sequences of H1 viruses from a linear time range, such as a linear time range or span (e.g., from the last 5, 10, 15, 20, 25, 30, 35, 40, 45, 50, 55, 60, 65, 70, 75, 80, 85, 90, 95, or 100 years) are assessed. In the practice of the method related to H1 viruses, the parameters of geography and species of the virus need not be assessed. A sequence identity range (usually from 2-5%) is employed, as described below.

Accordingly, in this embodiment, a method of generating a non-naturally occurring, broadly reactive, pan-epitopic immunogen capable of generating an immune response against present and future H1 strains in a subject is provided, in which the method comprises (a) generating a phylogenetic tree comprising full length related antigen sequences derived from H1 strains present during two or more consecutive flu seasons; identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; and (f) generating a phylogenetic tree comprising the secondary sequences, wherein branches of the tree are combined if the substitution rate per amino acid site distance is less than about 0.001, thereby generating a non-naturally occurring immunogen comprising a pan-epitopic immunogen sequence. In an embodiment, each cluster of antigen sequences in step (b) has at least 95%-98% sequence identity. In a certain embodiment of the method, the phylogenetic tree comprises sequences derived from flu strains present during the past 1-100 years. In a certain embodiment of the method, the phylogenetic tree comprises sequences derived from flu strains present during the past 5, 10, 20, 30, 40, 50, or 100 years.

In another embodiment, a method of obtaining a broadly reactive and immunogenic HA or NA antigen of an influenza H5 virus and viruses related thereto is provided. In the practice of the method, the parameters of HA or NA sequences obtained from a clade, geography, and species of virus are assessed. A sequence identity range (usually from 2-5%) is employed, as described below.

Accordingly, in this embodiment, a method of generating a non-naturally occurring, broadly reactive, pan-epitopic immunogen capable of generating an immune response against present and future H5 strains in a subject is provided, in which the method comprises (a) generating a phylogenetic tree comprising full length related antigen sequences derived from two or more H5 clades, or two or more H5 species, present in two or more selected geographic regions over one or more flu seasons; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; and (f) generating a phylogenetic tree comprising the secondary sequences, wherein branches of the tree are combined if the substitution rate per amino acid site distance is less than about 0.001, thereby generating a non-naturally occurring immunogen comprising a pan-epitopic immunogen sequence. In an embodiment, each cluster of antigen sequences in step (b) has at least 95%-98% sequence identity. In an embodiment, each cluster of antigen sequences in step (b) has at equal to or greater than 98% sequence identity.

In another embodiment, the method involves a consideration of the parameters of influenza virus antigen sequences, such as HA antigen sequences, from a time span or range (e.g., a linear time range), such as one or more flu seasons, and geographical location(s) in which the influenza virus was isolated, such as, for example, the Southern or Northern Hemisphere, wherein the steps of the method are as described hereinabove.

In an embodiment of the methods in the foregoing aspects, the antigen sequences are influenza virus HA, HA1, HA2, or NA antigen sequences.

In another aspect, a method of generating a non-naturally occurring, broadly reactive, pan-epitopic immunogen capable of generating an immune response against present and future Dengue virus strains in a subject is provided, in which the method comprises (a) generating a phylogenetic tree comprising full length related antigen sequences derived from Dengue strains present in the Americas or Asia during a selected period of time; (b) identifying clusters of antigen sequences within the tree, each cluster having at least 95% identity and at least about 0.001 substitution per site relative to the other sequences within the cluster; (c) generating for each cluster a non-naturally occurring primary sequence comprising amino acids that are conserved or identical within the cluster; (d) generating a phylogenetic tree comprising the primary sequences of step (c); (e) selecting three or more clusters of antigen sequences within the primary sequences, each selected cluster comprising at least about 0.001 amino acid substitutions per site and generating secondary sequences comprising amino acids that are conserved or identical within the three or more clusters; and (f) generating a phylogenetic tree comprising the secondary sequences, wherein branches of the tree are combined if the substitution rate per amino acid site distance is less than about 0.001, thereby generating a non-naturally occurring immunogen comprising a pan-epitopic immunogen sequence. In an embodiment, each cluster of antigen sequences in step (b) has at least 95%-98% sequence identity. In an embodiment, each cluster of antigen sequences in step (b) has at equal to or greater than 98% sequence identity.

In an embodiment of the above methods, step (d) and step (e) are optional. In an embodiment of the above methods, steps (a)-(c) are repeated two or more times. In an embodiment of the above methods, steps (b) and (c) are repeated two or more times prior to step (d). In an embodiment of the above methods, steps (e) and (f) are repeated two or more times. In another embodiment of the above methods, steps (a) through (e) are repeated two or more times prior to performing step (f).

In an embodiment of the above methods, three or more clusters of antigen sequences are identified in step (b). In a particular embodiment of the methods, 5 or 15 clusters of antigen sequences are identified in step (b).

In an embodiment of the above methods, the results or output generated by the practice of the methods, such as primary, secondary and tertiary sequences, sequence clusters, sequence alignments, phylogenetic trees, or any combinations thereof, are displayed in a visual form, such as displaying the results or output on a display device. In embodiments, the methods comprise displaying the generation of clusters of antigen sequences in a visual form, such as displaying on a display device. In other embodiments, the methods comprise displaying the generation of a phylogenetic tree comprising antigen sequences, such as the primary or secondary antigen sequences, in a visual form, such as displaying on a display device. In embodiments, the display device is a desktop computer, a laptop computer, a hand-held computer, a smart phone, a cellular telephone, a tablet computer, or a personal digital assistant.

In an embodiment of the above methods, the immunogen sequence generated by the practice of the method is expressed in a cell as a polypeptide, protein, or peptide. In an embodiment of the above methods, the immunogen generated by the practice of the method is isolated and/or purified. In an embodiment of the methods, the immunogen is formulated for administration to a subject in need. In an embodiment of the methods, the immunogen is administered to a subject in need thereof in an effective amount to elicit an immune response in the subject. In an embodiment of the described methods, the immune response elicits neutralizing antibodies. In an embodiment, the immune response is prophylactic or therapeutic.

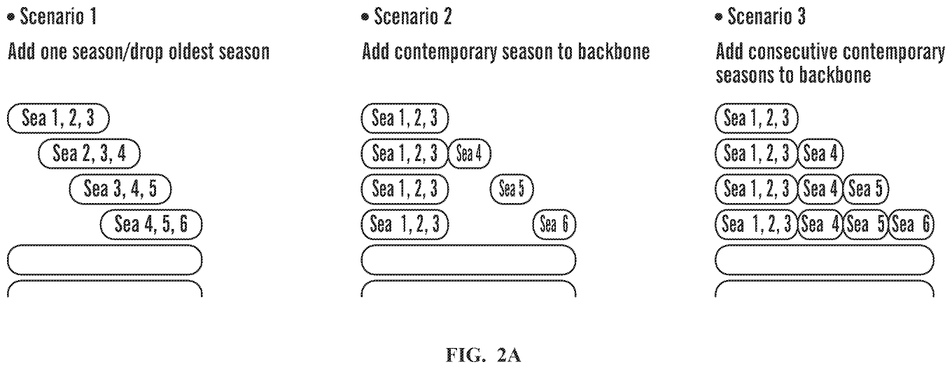

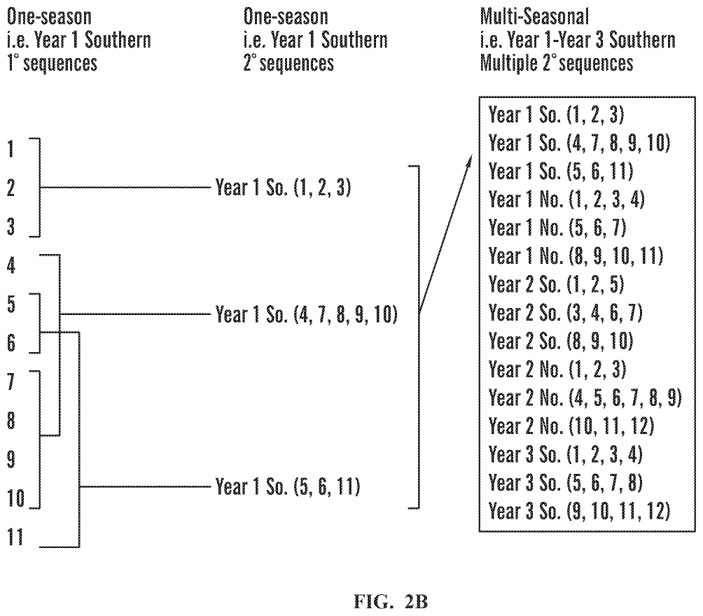

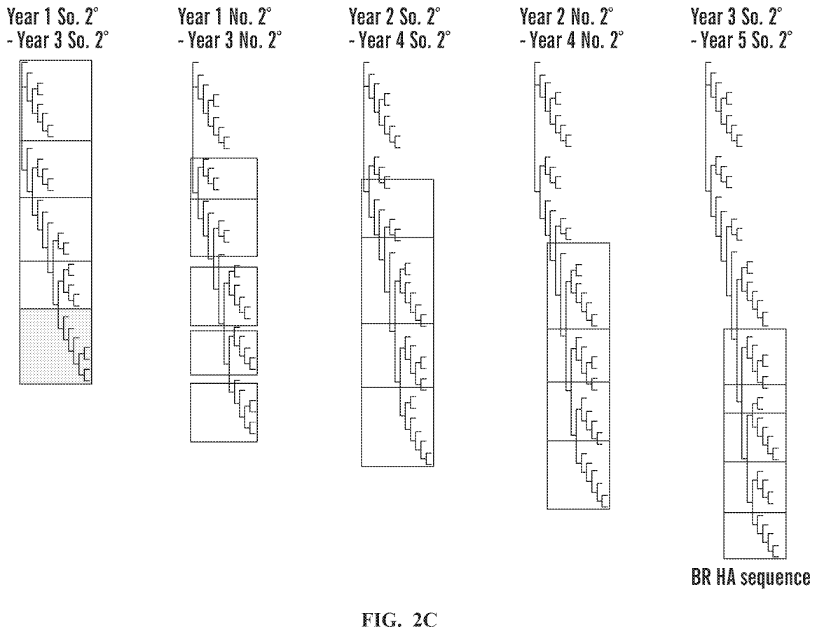

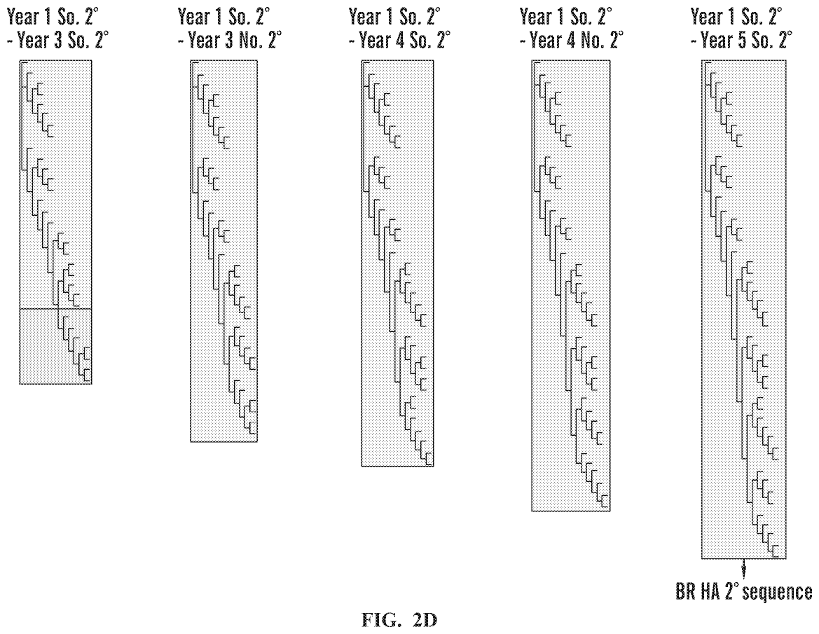

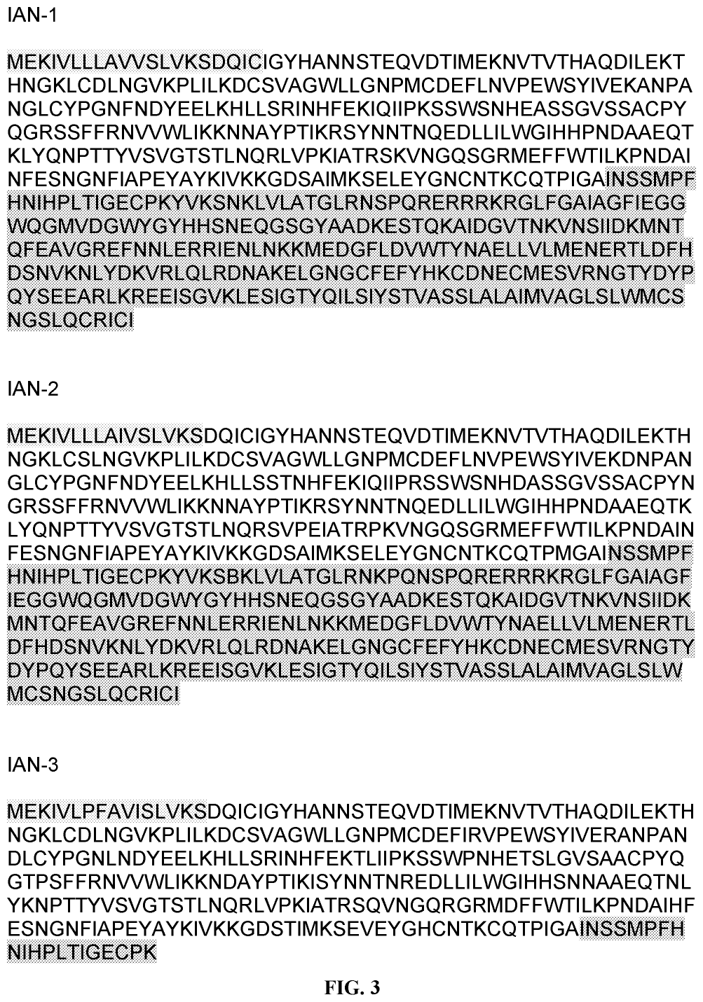

In other embodiments, the method may further involve a process to assess and include HA sequences from viruses (and virus clades or strains) in different geographical locations (e.g., Northern or Southern Hemisphere) collected over a given time period (e.g., spans of years, such as, without limitation, 3, 4, 5, 6, 7, 8 years, or longer, time spans, for example, as indicated in Step L of the method described in Example 1 herein. The further analysis of protein sequences over time spans and seasons, and the inclusion of antigen sequence variability in the analysis, provide a “rolling” or “seasonal rolling” effect, which augments the steps of the broadly reactive antigen production method and captures past and present antigen sequence similarity and variability. (FIG. 2A). Thus, in a “rolling” aspect of the method, HA protein sequences, for example, are included from past and present viruses having diversity in their HA sequences that are assessed, for example, over temporal spans, such as seasons of infection, in different geographic locations, thus allowing the inclusion of antigenic determinants that can result from virus drift variants and can capture rare or drifted antigenic determinants or epitopes within the resulting “pan-epitopic” antigen sequence generated by the method.

In one approach to a seasonal rolling methodology (Scenario 1), (FIGS. 2A-2C), sequence diversity is analyzed via phylogenetic analysis. By way of example, the process involves analyzing influenza virus HAI sequences between 20-300 amino acids in length, taking into account, for example, Southern Hemisphere season: May 1 XX-September 30 XX; Northern Hemisphere season: October 1 XX-April 30 XY; aligning the sequences; constructing Jukes-Cantor NJ phylogenetic tree and assigning clusters that contain 98-99% sequence identity such that each cluster yields a primary (1°) sequence, in which 12-20 clusters for each season are optimal; aligning the 1° sequences and constructing a tree to assess sequence similarity between clusters, in which identical 1° sequences are grouped together to make a single 2° sequence, wherein a minimum of 4-8 2° sequences is optimal; aligning the 2° sequences from multiple seasons (e.g., 5 seasons) into a phylogenetic tree and dividing the sequences in the tree into clusters, with a minimum of 3-2 2° sequences; and obtaining the sequences with similarity and/or identity to yield a tertiary (3°) sequence. (FIGS. 2B and 2C; Examples 1 and 2). It will be understood that the steps of the method, and/or the parameters and/or outcomes of the steps of the method, such as sequence clusters, phylogenetic trees, primary, secondary and tertiary sequence alignments generated during the practice of the method, may be displayed in a visual form, such as on a display device.

In some embodiments of any of the foregoing aspects, the method further includes (i) reverse translating the antigen sequence (e.g., virus protein antigen) generated by the method to generate a coding sequence; and (ii) optimizing the coding sequence for expression in mammalian cells.

In another embodiment of any of the foregoing methods, the immune response elicits neutralizing antibodies. In another embodiment of any of the foregoing methods, the immune response is prophylactic or therapeutic.

In embodiments of the foregoing methods, each identified cluster of antigen (amino acid) sequences within the tree has at least or equal to 93%, at least or equal to 94%, at least or equal to 95%, at least or equal to 96%, at least or equal to 97%, at least or equal to 98%, or at least or equal to 99% sequence identity and at least about 0.001 substitution per site relative to the other sequences within the cluster. In other embodiments, the sequence identity range is from about or equal to 94% to about or equal to 99%, or from about or equal to 95% to about or equal to 98%, or from about or equal to 96% to about or equal to 98%.

Display of Results and Output, and/or Parameters, of Method Steps in a Visual Form, Such as on a Display Device

In an aspect related to the described methods, a non-transitory computer readable medium containing program instructions executable by a processor is provided, in which the computer readable medium contains program instructions that provide criteria that relate one or more parameters to each other, the parameters including one or more selected from the group consisting of: sequence gathering parameters, sequence alignment parameters, computationally-derived parameters of clustered sequence output and phylogenetic tree generation (related to type/subtype of pathogen, sequence identity and variability); and program instructions that input parameters of the method steps into given criteria and relate observations for one or more acquired parameters; and program instructions that converge the given criteria so as to provide an output representative of sequences containing both similarity and variability, so as to represent broadly reactive epitopes/antigenic determinants.

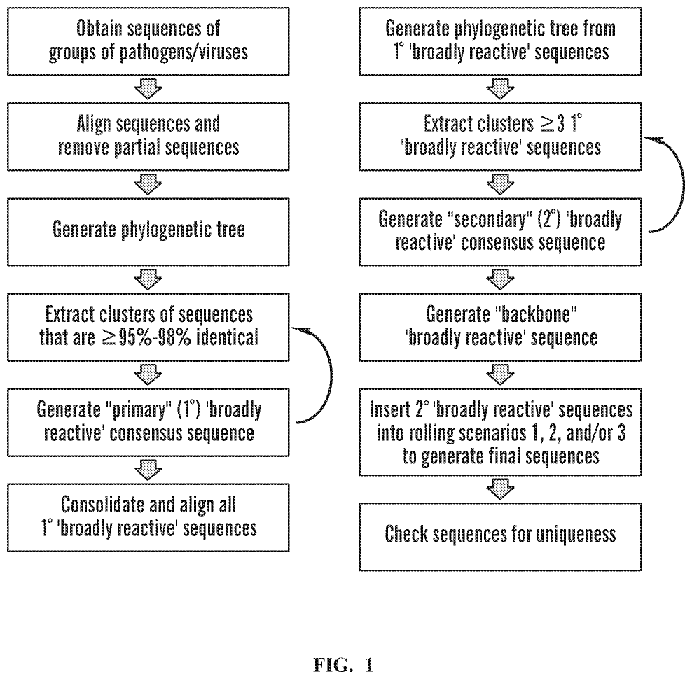

As described herein, FIG. 1 depicts a schematic flow diagram of the described method for generating non-naturally occurring, broadly reactive, pan-epitopic antigen sequences derived from a pathogen such as a virus, e.g., influenza virus of different types and subtypes. In an aspect, the method continues with displaying the input, output, and results of inputted data in one or more, or all, of the steps of the method so that one can visualize antigen sequence relationships (sequence identity and sequence variability), clustering of antigen sequences from pathogens (e.g., different clades, types, or subtypes) present in different time spans or periods (e.g., seasons) and different geographies (e.g., Northern or Southern Hemisphere). The flow chart also illustrates the structure of the logic of the different methods, which can be embodied in computer program software for execution on a computer, digital processor, or microprocessor. Those skilled in the art will appreciate that the flow chart illustrates the structure of the computer program code elements, including logic circuits on an integrated circuit that function according to the described methods. As such, one or more, or all, of the steps of the method may be practiced, with human input and involvement, by a machine component that renders the program code elements in a form that instructs a digital processing apparatus (e.g., computer) to perform a sequence of function step(s) corresponding to those shown in the flow diagram.

Accordingly, the methods as described herein are suitable for use in combination with any of a number of computer systems as are known to those skilled in the art or hereinafter developed. Such a computer system typically includes a computer, a display, and one or more input device(s). The display is any of a number of devices known to those skilled in the art for displaying images responsive to output signals from the computer, including but not limited to cathode ray tubes (CRT), liquid crystal displays (LCDS), plasma screens and the like. The signals being outputted from the computer can originate from any of a number of devices including PCI or AGP video boards or cards mounted with the housing of the computer that are operably coupled to the computer's microprocessor and the display.

The one or more input device(s) are any of a number of devices known to those skilled in the art that can be used to provide input signals to the computer for control of applications programs and other programs such as the operating system (OS) being executed within the computer. Illustratively, the input device may comprise a switch, a slide, a mouse, a track ball, a glide point or a joystick, or other such device (e.g., a keyboard having an integrally mounted glide point or mouse) by which a user can input control signals other than by means of a keyboard.

The computer typically includes a central processing unit (CPU) including one or more microprocessors such as those manufactured by Intel or AMD, Motorola or the like, random access memory (RAM), mechanisms and structures for performing I/O operations, a storage medium such as a magnetic hard disk drive(s) or other drives (fixed or removable) for storage of data, operating systems or the applications or software programs associated with the methods, including an applications program and a device for reading from and/or writing to a removable computer readable medium, such as, for example, an optical disk reader capable of reading CDROM, DVD or optical disks and readers of other types of nonvolatile memory, such as flash drives, jump drives, or spin memory that embody one or more types of non-volatile memory or storage devices.

Such a hard disk drive serves to boot or store the operating system, other applications, or systems that are to be executed on the computer, paging and swapping between the hard disk and the RAM and the like. Such data also can be stored in a removable computer readable medium such as a CD or DVD type of medium that is inserted into a device for reading and/or writing to the removable computer readable media. Alternatively, such a computer system also includes a network based computer system that includes a server, an external storage device and a network infrastructure that operably couples a plurality or more of client computer systems to the server.

The server is any of a number of servers known to those skilled in the art that are intended to be operably connected to a network so as to operably link a plurality of client computers via the network to the server and thus also to the external storage device. Such a server typically includes a central processing unit including one or more microprocessors such as those manufactured by Intel or AMD, random access memory (RAM), mechanisms and structures for performing I/O operations, a storage medium such as a magnetic hard disk drive(s), and an operating system for execution on the central processing unit.

Devices suitable for the display of data generated by the described methods include desktop computers, laptop computers, hand held computer devices, smart phone, cellular telephone, tablet computer, or personal digital assistant. Such devices typically have an OS capable of running application software (e.g., Apps) and also typically provide for wireless connection to the Internet (e.g., WI-FI, Bluetooth).

### Immunogenic Antigens Obtained Through the Practice of the Methods

The practice of the described methods generates a pathogen-derived, pan-epitopic antigen sequence (antigen) which is a non-naturally occurring immunogen that elicits a broadly reactive immune response in a subject following introduction, administration, or delivery of the immunogen to the subject. The route of introduction, administration, or delivery is not limited and may include, for example, intravenous, subcutaneous, intramuscular, oral, etc. routes. In an embodiment, a vaccine comprising the non-naturally occurring immunogen generated by the practice of any of the foregoing methods is provided. The vaccine may be therapeutic (e.g., administered to a subject following a symptom of disease caused by a pathogen) or prophylactic (protective), (e.g., administered to a subject prior to the subject having or expressing a symptom of disease, or full-blown disease, caused by a pathogen).

In an embodiment, the final amino acid sequence of the antigen, e.g., HA or NA, arrived at through the practice of the methods is reverse translated and optimized for expression in mammalian cells. As will be appreciated by the skilled practitioner in the art, optimization of the nucleic acid sequence includes optimization of the codons for expression of a sequence in mammalian cells and RNA optimization (such as RNA stability).

In an embodiment, an isolated nucleic acid molecule (polynucleotide) comprising a nucleotide sequence encoding a polypeptide or peptide antigen, such as an influenza HA polypeptide (or HAI or HA2 polypeptide), generated by the described methods is provided. In certain embodiments, the nucleotide sequence encoding the HA polypeptide is at least 94%, at least 95%, at least 96%, at least 97%, at least 98% or at least 99% identical to a polynucleotide encoding an HA polypeptide (or HAI or HA2 polypeptide) sequence shown in FIG. 3.

In other embodiments, the nucleotide sequence encoding the influenza HA polypeptide (or HAI or HA2 polypeptide) that is at least 90%, at least 91%, at least 92%, at least 93%, at least 94%, at least 95%, at least 96%, at least 97%, at least 98%, or at least 99% identical to a polynucleotide encoding an HA polypeptide (or HAI or HA2 polypeptide) sequence shown in FIG. 3 lacks the start codon encoding an N-terminal methionine.

Vectors containing a nucleotide sequence encoding a non-naturally occurring, broadly reactive polypeptide or peptide antigen, such as an influenza HA polypeptide, (or HAI or HA2 polypeptide), obtained by the described methods are provided. In some embodiments, the vectors comprise a nucleotide sequence encoding the polypeptide or peptide antigen, such as an influenza HA polypeptide antigen, that is at least 94%, at least 95%, at least 96%, at least 97%, at least 98% or at least 99% identical to a polynucleotide encoding an HA polypeptide (or HAI or HA2 polypeptide) sequence shown in FIG. 3. In some embodiments, the vector further includes a promoter operably linked to the nucleotide sequence encoding the HA polypeptide (or HAI or HA2 polypeptide). In a particular embodiment, the promoter is a cytomegalovirus (CMV) promoter. In some embodiments, the nucleotide sequence of the vector is at least 85%, at least 90%, at least 95%, at least 98% or at least 99% identical to a polynucleotide encoding an HA polypeptide (or HAI or HA2 polypeptide) sequence shown in FIG. 3. In particular embodiments, the nucleotide sequence of the vector comprises the polynucleotide encoding an HA polypeptide (or HAI or HA2 polypeptide) sequence shown in FIG. 3. In embodiments, the vector is a prokaryotic or eukaryotic vector. In an embodiment, the vector is an expression vector, such as a eukaryotic (e.g., mammalian) expression vector. In another embodiment, the vector is a plasmid (prokaryotic or bacterial) vector. In another embodiment, the vector is a viral vector.

The vectors used to express antigens generated by the described methods, e.g., viral proteins, such as the HA, NA and M1 proteins, may be any suitable expression vectors known and used in the art. The vectors can be, for example, mammalian expression vectors or viral vectors. In some embodiments, the vector is the pTR600 expression vector (U.S. Patent Application Publication No. 2002/0106798, herein incorporated by reference; Ross et al., 2000, Nat Immunol 1(2):102-103; and Green et al., 2001, Vaccine 20:242-248).

Provided are pathogen-derived, non-naturally occurring polypeptide antigens, e.g., influenza HA polypeptide antigens, or HAI or HA2 polypeptide antigens, obtained by the described methods and produced by transfecting a host cell with an expression vector as known and used in the art under conditions sufficient to allow for expression of the polypeptide, e.g., an HA, HA1, or HA2 polypeptide, in the cell. Isolated cells containing the vectors are also provided.

Also provided are non-naturally occurring, broadly reactive, pan-epitopic antigen polypeptides generated by the methods described herein, such as pan-epitopic, broadly reactive influenza HA (or NA) polypeptides. In certain embodiments, the amino acid sequence of the polypeptide is at least 95% to 99% (inclusive) identical to the amino acid sequence of an HA, HA1, or HA2 polypeptide produced by the described methods and shown in FIG. 3. In particular embodiments, the amino acid sequence of the influenza HA, HA1, or HA2 polypeptide that is at least 95% to 99% (inclusive) identical to the amino acid sequence of an HA, HA1, or HA2 polypeptide shown in FIG. 3 lacks the N-terminal methionine residue. In a particular embodiment, the amino acid sequence of the influenza HA polypeptide is at least 95% to 99% (inclusive) identical to amino acids 1-566 of the HA, HA1, or HA2 polypeptides shown in FIG. 3.

In some embodiments, fusion proteins comprising the broadly reactive, pan-epitopic antigen polypeptides generated by the methods described herein, e.g., without limitation, the influenza HA polypeptides disclosed herein, are also provided. In some embodiments, the influenza HA polypeptide can be fused to any heterologous amino acid sequence to form the fusion protein. By way of example, HAI and HA2 polypeptides may be generated independently and then fused together to produce an HA polypeptide antigen, e.g., comprising 566 amino acids.

Also provided are virus-like particles (VLPs), in particular, influenza VLPs, containing a pan-epitopic, broadly reactive protein antigen, e.g., influenza HA, HA1, or HA2 protein, as described herein. In certain embodiments, the HA protein of the VLP is at least or equal to 94%, at least or equal to 95%, at least or equal to 96%, at least or equal to 97%, at least or equal to 98%, at least or equal to 99% or 100% identical to the HA, HA1, or HA2 proteins as shown in FIG. 3. The virus or influenza VLPs can further include any additional viral or influenza proteins necessary to form the virus particle. In certain embodiments, the virus or influenza VLPs further include influenza neuraminidase (NA) protein, influenza matrix (M1) protein, or both.

In some embodiments, the amino acid sequence of the virus or influenza NA protein is at least or equal to 85%, at least or equal to 90%, at least or equal to 95%, at least or equal to 98% or at least or equal to 99% identical to an NA protein sequence shown in FIG. 4. In some embodiments, the amino acid sequence of the influenza NA protein comprises an NA protein sequence shown in FIG. 4.

Also provided is an influenza VLP containing an influenza HA, HA1, or HA2 polypeptide as described herein, produced by transfecting a host cell with a vector encoding the HA, HA1, or HA2 polypeptide. Also provided in an embodiment is an influenza VLP containing an influenza HA polypeptide, or HAI or HA2 polypeptide, as described herein, produced by transfecting a host cell with a vector encoding the HA, HA1, or HA2 polypeptide, a vector encoding an influenza NA protein and a vector encoding an influenza M1 protein, under conditions sufficient to allow for expression of the HA, NA and M1 proteins.

Collections of plasmids (vectors) are also contemplated. In certain embodiments, the collection of plasmids includes a plasmid encoding an influenza NA, a plasmid encoding an influenza MA, and a plasmid encoding a pan-epitopic, broadly reactive HA, HAI or HA2 protein produced by the methods as described herein. In some embodiments, the nucleotide sequence encoding a codon-optimized influenza HA, HA1, or HA2 of the HA-encoding plasmid is at least 94%, at least 95%, at least 96%, at least 97%, at least 98% or at least 99% identical to a polynucleotide encoding an HA, HA1, or HA2 amino acid sequence as shown in FIG. 3.

In some embodiments, the influenza NA is codon-optimized and/or the influenza M1 is codon-optimized in the collections of plasmids. In some examples, the nucleotide sequence encoding the codon-optimized influenza NA is at least or equal to 85%, at least or equal to 90%, at least or equal to 95%, at least or equal to 96%, at least or equal to 97%, at least or equal to 98% or at least or equal to 99% identical to a polynucleotide sequence encoding an NA polypeptide sequence as shown in FIG. 4. In particular examples, the nucleotide sequence encoding the codon-optimized influenza NA antigen protein comprises, or consists of, the polynucleotide sequence encoding the NA proteins shown in FIG. 4.

### Methods of Producing a Pan-Epitopic, Broadly Reactive Pathogen (e.g., Influenza Virus)-Derived Protein Sequence, Such as an Influenza Protein Sequence

In an embodiment, a method of producing a pan-epitopic, broadly reactive pathogen-derived protein sequence, such as an influenza protein sequence, to elicit a broadly reactive immune response in a subject is provided. In the context of the present disclosure, “broadly reactive” or “broadly active” means that the protein (e.g., the protein sequence, such as an HA, HA1, HA2, or NA protein) is immunogenic and contains a diversity of epitopes (antigenic determinants; pan-epitopic) that elicit in a subject an immune response (e.g., neutralizing antibodies directed against the diversity of epitopes, frequently accompanied by a T-cell response) sufficient to treat disease or infection, and/or to inhibit, neutralize, or prevent infection, caused by a pathogen, e.g., caused by a broad range of influenza viruses (such as most or all influenza viruses within a specific subtype). In some embodiments, the influenza protein is influenza HA or influenza NA. In embodiments, the broadly reactive, pathogen-derived antigen protein, e.g., influenza protein, is capable of eliciting a protective immune response against most or all known influenza virus isolates, such as about 80%, about 85%, about 90%, about 95%, or about 96%-99% of known influenza virus isolates, e.g., the H1, H5, H5N1, H7, H9 isolates.

In some embodiments, the method of generating a broadly reactive, pan-epitopic protein sequence derived from a pathogen, for example, an influenza protein sequence, includes obtaining the amino acid sequences of a group of pathogens, e.g., influenza virus, isolates. In the case of influenza, for example, the group can contain influenza virus isolates from a specific subtype (such as, for example, H5N1 or H1N1), and/or from one or more clades/sub-clades of a specific influenza subtype (for example, one or more of clades/sub-clades 0, 1, 2.1, 2.2, 2.3, 2.4, 3, 4, 5, 6, 7, 8 and 9 of H5N1). In some embodiments, and in accordance with the methods described supra, amino acid sequences of the group of influenza viruses are first organized by clade or sub-clade and then by geographic location within each clade or sub-clade.

The amino acid sequences having sequence similarity and/or identity for each geographic location are aligned to generate a primary sequence for each geographical region. Grouping virus isolates by geographical region controls for single outbreak dominance and incomplete reporting and sequencing. A primary sequence can be generated, for example, by performing sequence analysis using AlignX (Vector NTI), or by any other method known in the art. The primary geographically-based sequences (e.g., for each clade or sub-clade) are then aligned, phylogenetic clusters of sequences are generated, followed by the generation of a secondary sequence, e.g., for each clade or sub-clade. The secondary sequences (e.g., for each virus clade or sub-clade, if applicable) are then aligned to generate the pan-epitopic, broadly reactive, sequence in accordance with the method (see, e.g., FIG. 1). In a particular embodiment, a broadly reactive, pan-epitopic influenza virus polypeptide sequence is optimized for expression in mammalian cells, for example, by reverse translation of the broadly reactive influenza virus polypeptide sequence to generate a coding sequence, followed by codon-optimization and/or optimization of the RNA (such as for stability).

### Compositions and Pharmaceutical Compositions for Administration

Broadly reactive, pan-epitopic antigen proteins derived from a number of different pathogens, or a composition comprising such a protein produced according to the methods described herein is provided. In a certain embodiment, a broadly reactive, pan-epitopic influenza HA (or NA) protein, or a fusion protein or VLP comprising such an influenza HA protein produced by the methods described herein, or compositions comprising the foregoing, is provided. In some embodiments, the compositions further comprise a pharmaceutically acceptable carrier, excipient, or vehicle. In some embodiments, an adjuvant (a pharmacological or immunological agent that modifies or boosts an immune response, e.g. to produce more antibodies that are longer-lasting) is also employed. For example, without limitation, the adjuvant can be an inorganic compound, such as alum, aluminum hydroxide, or aluminum phosphate; mineral or paraffin oil; squalene; detergents such as Quil A; plant saponins; Freund's complete or incomplete adjuvant, a biological adjuvant (e.g., cytokines such as IL-1, IL-2, or IL-12); bacterial products such as killed Bordetella pertussis, or toxoids; or immunostimulatory oligonucleotides (such as CpG oligonucleotides).

Compositions and preparations (e.g., physiologically or pharmaceutically acceptable compositions) containing the non-naturally occurring, broadly reactive, pan-epitopic, pathogen-derived antigen polypeptides, such as influenza HA or NA polypeptides and influenza virus-like particles (VLPs), for parenteral administration include, without limitation, sterile aqueous or non-aqueous solutions, suspensions, and emulsions, such as used in the art. See, e.g., Remington, Essentials of Pharmaceutics, Royal Pharmaceutical Society, Pharmaceutical Press, 2013 (or later edition). Nonlimiting examples of non-aqueous solvents include propylene glycol, polyethylene glycol, vegetable oils, such as olive oil and canola oil, and injectable organic esters, such as ethyl oleate. Aqueous carriers, excipients, diluents, or vehicles include water, alcoholic/aqueous solutions, emulsions or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanol amines.

Provided herein are pharmaceutically acceptable compositions which include a therapeutically effective amount of a non-naturally occurring, broadly reactive, pan-epitopic, pathogen-derived antigen polypeptides generated by the described methods, such as virus protein antigens, or influenza VLPs, alone or in combination with a pharmaceutically acceptable carrier, excipient, diluent, or vehicle. Pharmaceutically acceptable carriers, excipient, diluents, or vehicles include, but are not limited to, saline, buffered saline, dextrose, water, glycerol, ethanol, and combinations thereof. The carrier, etc. and composition can be sterile, and the formulation is prepared using commonly known techniques to suit the mode of administration. The compositions can also contain minor amounts of wetting or emulsifying agents, or pH buffering agents. The compositions can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used in the compositions and administration methods as described are normal saline and sesame oil.

### Methods of Treatment, Administration and Delivery

Methods of treating a disease or infection, or symptoms thereof, caused by a pathogenic microorganism such as a bacterium or virus (e.g., influenza virus) are provided. The methods comprise administering a therapeutically effective amount of a broadly reactive, pan-epitopic immunogen produced by the methods described herein, or a pharmaceutical composition comprising the immunogen, or a vaccine (e.g., a VLP vaccine) as described herein, to a subject (e.g., a mammal), in particular, a human subject. In an embodiment, more than one broadly reactive immunogen may be components in an immunogenic composition. One embodiment involves a method of treating a subject suffering from, or at risk of or susceptible to disease, infection, or a symptom thereof, caused by a pathogen, e.g., bacterial or virus infection, particularly influenza virus infection. The method includes administering to the subject (e.g., a mammalian subject), an amount or a therapeutic amount of an immunogenic composition or a vaccine comprising a non-naturally occurring, broadly reactive, pan-epitopic, pathogen-derived antigen polypeptide, such as an influenza HA, HA1, HA2, or NA polypeptide, or such polypeptide VLPs, sufficient to treat the disease, infection, or symptoms thereof, caused by the pathogen, such as influenza virus, under conditions in which the pathogen-associated or virus-associated disease, infection, and/or the symptoms thereof are treated.

In an embodiment, the methods herein include administering to the subject (including a human subject identified as being in need of such treatment) an effective amount of a non-naturally occurring, broadly reactive, pan-epitopic, pathogen-derived antigen polypeptide, such as virus HA or NA polypeptide, or a vaccine, or a composition as described herein to produce such effect. The treatment methods are suitably administered to subjects, particularly humans, suffering from, having, susceptible to, or at risk of having a disease, disorder, infection, or symptom thereof. Identifying a subject in need of such treatment can be based on the judgment of the subject or of a health care professional and can be subjective (e.g. opinion) or objective (e.g. measurable by a test or diagnostic method). Briefly, the determination of those subjects who are in need of treatment or who are “at risk” or “susceptible” can be made by any objective or subjective determination by a diagnostic test (e.g., genetic test, enzyme or protein marker assay), marker analysis, family history, and the like, including an opinion of the subject or a health care provider. The non-naturally occurring, broadly reactive, pan-epitopic, pathogen-derived antigen polypeptides, such as influenza virus HA or NA polypeptides and vaccines as described herein, may also be used in the treatment of any other disorders in which infection or disease caused by the pathogenic microorganism, in particular, a virus such as influenza virus, may be implicated. A subject undergoing treatment can be a non-human mammal, such as a veterinary subject, or a human subject (also referred to as a “patient”).

In addition, prophylactic methods of preventing or protecting against a disease or infection, or symptoms thereof, caused by a pathogenic microorganism, such as a virus (e.g., influenza virus) are provided. Such methods comprise administering a therapeutically effective amount of a pharmaceutical composition comprising a vaccine (e.g., a VLP vaccine) as described herein to a subject (e.g., a mammal such as a human), in particular, prior to infection of the subject, or prior to onset of the disease, such as pathogen-associated disease, or virus-associated disease.

In another embodiment, a method of monitoring the progress of a disease or infection associated with or caused by a pathogen (such as a microorganism pathogen), or by a virus, or monitoring treatment of the disease or infection is provided. The method includes determining a level of a diagnostic marker or biomarker (e.g., a pathogen surface protein or a virus protein such as HA or NA), or a diagnostic measurement (e.g., screening assay or detection assay) in a sample from a subject suffering from or susceptible to infection, disease, or symptoms thereof associated with a pathogen, such as a virus, e.g., influenza, in which the subject has been administered an amount (e.g., a therapeutic amount) of a non-naturally occurring, broadly reactive, pan-epitopic, pathogen-derived antigen polypeptide or virus HA protein generated according to the methods described herein, or a vaccine as described herein sufficient to treat the infection, disease, or symptoms thereof. The level or amount of the marker or biomarker (e.g., protein) determined in the method can be compared to known levels of the marker or biomarker in samples from healthy, normal controls; in a pre-infection or pre-disease sample of the subject; or in other afflicted/infected/diseased patients to establish the treated subject's disease status. For monitoring, a second level or amount of the marker or biomarker in in a sample obtained from the subject is determined at a time point later than the determination of the first level or amount, and the two marker or biomarker levels or amounts can be compared to monitor the course of disease or infection, or the efficacy of the therapy/treatment. In certain embodiments, a pre-treatment level of the marker or biomarker in the subject (e.g., in a sample obtained from the subject) is determined prior to beginning treatment as described; this pre-treatment level of marker or biomarker can then be compared to the level of the marker or biomarker in the subject after the treatment commences and/or during the course of treatment to determine the efficacy of (monitor the efficacy of) the disease treatment.

The non-naturally occurring, broadly reactive, pan-epitopic, pathogen-derived antigen polypeptides, such as influenza HA polypeptides generated in accordance with the described methods, and VLPs comprising HA polypeptides, or compositions thereof, can be administered to a subject by any of the routes normally used for introducing a recombinant protein, composition containing the recombinant protein, or recombinant virus into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, or oral. Parenteral administration, such as subcutaneous, intravenous or intramuscular administration, is generally achieved by injection. Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local.

The non-naturally occurring, broadly reactive, pan-epitopic, pathogen-derived antigen polypeptides, such as influenza HA polypeptides generated in accordance with the described methods, and VLPs comprising HA polypeptides, or compositions thereof, can be administered in any suitable manner, such as with pharmaceutically acceptable carriers as described supra. Pharmaceutically acceptable carriers are determined in part by the particular immunogen or composition being administered, as well as by the particular method used to administer the composition. Accordingly, a pharmaceutical composition comprising the non-naturally occurring, broadly reactive, pan-epitopic, pathogen-derived antigen polypeptides, such as influenza HA polypeptides, and VLPs comprising HA polypeptides, or compositions thereof, can be prepared using a wide variety of suitable and physiologically and pharmaceutically acceptable formulations.

Administration of the broadly reactive, pan-epitopic, pathogen-derived antigen polypeptides, such as influenza HA polypeptides, generated in accordance with the described methods, and VLPs comprising HA polypeptides, or compositions thereof, can be accomplished by single or multiple doses. The dose administered to a subject should be sufficient to induce a beneficial therapeutic response in a subject over time, such as to inhibit, reduce, ameliorate, or prevent disease or infection by a pathogen, such as a bacterial or virus, e.g., H1, H5, H7, or H9 influenza virus, infection. The dose required will vary from subject to subject depending on the species, age, weight and general condition of the subject, by the severity of the infection being treated, by the particular composition being used and by the mode of administration. An appropriate dose can be determined by a person skilled in the art, such as a clinician or medical practitioner, using only routine experimentation.

Further provided is a method of eliciting an immune response to a pathogen, such as influenza virus, in a subject, by administering to the subject a non-naturally occurring, broadly reactive, pan-epitopic, pathogen-derived antigen polypeptide generated in accordance with the described methods as immunogen, or a composition comprising the immunogen. In a particular embodiment, influenza HA polypeptides generated in accordance with the described methods, and VLPs comprising HA polypeptides, or compositions thereof are administered to the subject. In the case of influenza virus, by way of example, the influenza virus is an H1, H5, H7, or H9 type of influenza virus. In particular embodiments, the influenza virus is H1N1 or H5N1 virus. In some embodiments, the HA protein, HA fusion protein or VLP can be administered using any suitable route of administration, such as, for example, by intramuscular injection. In some embodiments, the HA protein, fusion protein, or VLP is administered as a composition comprising a pharmaceutically acceptable carrier. In some embodiments the composition comprises an adjuvant selected from, for example, alum, Freund's complete or incomplete adjuvant, a biological adjuvant or immunostimulatory oligonucleotides (such as CpG oligonucleotides). In other embodiments, the composition may be administered in combination with another therapeutic agent or molecule.

Also provided is a method of immunizing a subject against infection or disease or the symptoms thereof caused by a pathogen, such as influenza virus. In some cases, the method involves administering to the subject VLPs containing a non-naturally occurring, pan-epitopic, broadly reactive influenza HA protein generated according to the described methods, or administering a composition thereof. In some embodiments of the method, the composition further comprises a pharmaceutically acceptable carrier and/or an adjuvant. For example, the adjuvant can be alum, Freund's complete or incomplete adjuvant, a biological adjuvant or immunostimulatory oligonucleotides (such as CpG oligonucleotides). In an embodiment, the VLPs (or compositions thereof) are administered intramuscularly.

In some embodiments of the methods of eliciting an immune response or immunizing a subject against infection or disease caused by or associated with a pathogen, such as a virus, e.g., influenza virus, the subject is administered at least 1 μg of the VLPs containing a non-naturally occurring, broadly reactive, pan-epitopic HA or NA protein, such as at least 5 at least 10 at least 15 at least 20 at least 25 at least 30 at least 40 or at least 50 μg of the VLPs containing the HA or NA protein, for example about 1 to about 50 μg or about 1 to about 25 μg of the VLPs containing the non-naturally occurring, broadly reactive, pan-epitopic HA or NA protein. In particular examples, the subject is administered about 5 to about 20 μg of the VLPs, or about 10 to about 15 μg of the VLPs. In a specific, yet nonlimiting example, the subject is administered about 15 μg of the VLPs. However, one of skill in the art is capable of determining a therapeutically effective amount of VLPs (for example, an amount that provides a therapeutic effect or protection against the pathogen, e.g., H1N1 or H5N1 influenza virus infection) suitable for administering to a subject in need of treatment or protection from virus infection.

It is expected that the administration of immunogens, immunogenic compositions and VLPs comprising a non-naturally occurring, broadly reactive, pan-epitopic HA or NA protein generated by the methods described herein will elicit high titers of neutralizing antibodies directed against the diverse repertoire of epitopic determinants on the HA or NA protein immunogen, as well as protective levels of HA- or NA-inhibiting (such as HAI) antibodies that are directed against a number of representative virus clade isolates and will provide complete protection against lethal challenge with, for example, an H1N1 or H5N1 virus of different clades. By way of nonlimiting example, in some embodiments, the administration of VLPs containing a broadly reactive, pan-epitopic influenza HA protein results in the production of high HAI antibody titers (1:40) to H1, H5, H7, or H9 viruses, such as H5N1 clade 1, clade 2.1, clade 2.2 and clade 2.3 isolates. In some cases, the VLPs containing a broadly reactive, pan-epitopic influenza HA protein elicit high HAI titers against clade 1 and/or clade 7 viruses. The immunogens, immunogenic compositions and VLPs containing a non-naturally occurring, broadly reactive, pan-epitopic influenza HA protein (immunogen) generated by the methods as described herein elicit a broader immune response (e.g., elicit neutralizing antibodies directed against a broader range of virus isolates of a given virus type, e.g., without limitation, H5N1 virus from clade 1, sub-clades of clade 2, and clade 7) compared to the immune response elicited by a polyvalent influenza virus vaccine, which typically contains a preparation of two or more strains, types, subtypes, or isolates of the virus.

Accordingly, the non-naturally occurring, broadly reactive, pan-epitopic immunogens provided by the methods described herein provide a beneficial advantage over polyvalent vaccine preparations, both in terms of treatment and cost effectiveness. By their pan-epitopic nature, the broadly reactive pathogen-derived or virus-derived antigens and antigen sequences generated by the methods described herein provide a specific, unique immunogen, immunogenic composition, or vaccine for eliciting an immune response against different pathogen or virus types and subtypes (and related pathogens or viruses) in a recipient subject. This stands in contrast to polyvalent or multivalent vaccines which are typically prepared from two or more (multiple) strains, isolates, or subtypes of pathogens, such as viruses, e.g., H1, H5, H7, or H9, which do not provide the diversity and universality of antigenic determinants (epitopes) as provided by a specifically-generated immunogen described herein, and which are more labor intensive and costly to produce compared with an immunogen generated by the presently described methods.

### Adjuvants and Combination Therapies

The immunogens or immunogenic compositions containing a pathogen-derived antigen generated by the described methods, or containing influenza VLPs as described herein, can be administered alone or in combination with other therapeutic agents, drugs, or compounds, to enhance antigenicity or immunogenicity, i.e., to increase an immune response, such as the elicitation of specific antibodies or a memory response, in a subject. For example, the compositions or influenza VLPs can be administered with an adjuvant, such as alum, Freund's incomplete adjuvant, Freund's complete adjuvant, a biological adjuvant, or an immunostimulatory oligonucleotides (such as CpG oligonucleotides).

One or more cytokines, such as interleukin-1 (IL-2), interleukin-6 (IL-6), interleukin-12 (IL-12), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), or interferon-gamma (IFN-γ); one or more growth factors, such as GM-CSF or granulocyte-colony stimulation factor (G-CSF); one or more molecules such as the TNF ligand superfamily member 4 ligand (OX40L) or the type 2 transmembrane glycoprotein receptor belonging to the TNF superfamily (4-1BBL), or combinations of these molecules, can be used as biological adjuvants, if desired or warranted (see, e.g., Salgaller et al., 1998, J. Surg. Oncol. 68(2):122-38; Lotze et al., 2000, Cancer J. Sci. Am. 6(Suppl 1):S61-6; Cao et al., 1998, Stem Cells 16(Suppl 1):251-60; Kuiper et al., 2000, Adv. Exp. Med. Biol. 465:381-90). These molecules can be administered systemically (or locally) to a subject.

Several ways of inducing cellular responses, both in vitro and in vivo, are known and practiced in the art. Lipids have been identified as agents capable of assisting in priming cytotoxic lymphocytes (CTL) in vivo against various antigens. For example, palmitic acid residues can be attached to the alpha and epsilon amino groups of a lysine residue and then linked (for example, via one or more linking residues, such as glycine, glycine-glycine, serine, serine-serine, or the like) to an immunogenic peptide (U.S. Pat. No. 5,662,907). The lipidated peptide can then be injected directly in a micellar form, incorporated in a liposome, or emulsified in an adjuvant. As another example, E. coli lipoproteins, such as tripalmitoyl-S-glycerylcysteinlyseryl-serine can be used to prime tumor-specific CTL when covalently attached to an appropriate peptide (see, e.g., Deres et al., 1989, Nature 342:561). Moreover, the induction of neutralizing antibodies can also be primed with the same molecule conjugated to a peptide which displays an appropriate epitope, and two compositions can be combined to elicit both humoral and cell-mediated responses where such a combination is deemed desirable.

While treatment methods may involve the administration of VLPs containing a non-naturally occurring, broadly reactive, pan-epitopic HA or NA immunogenic protein generated by the methods described herein, one skilled in the art will appreciate that the non-naturally occurring, broadly reactive, pan-epitopic influenza HA or NA protein itself (in the absence of a viral particle), as a component of a pharmaceutically acceptable composition, or as a fusion protein, can be administered to a subject in need thereof to elicit an immune response in the subject.

### Kits

Also provided are kits containing a pathogen-derived, non-naturally occurring, broadly reactive immunogen generated by the described methods, or a vaccine, or a pharmaceutically acceptable composition containing the immunogen and a pharmaceutically acceptable carrier, diluent, or excipient, for administering to a subject, for example. Kits containing one or more of the plasmids, or a collection of plasmids as described herein, are also provided. As will be appreciated by the skilled practitioner in the art, such a kit may contain one or more containers that house the immunogen, vaccine, or composition, diluents or excipients, as necessary, and instructions for use.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

The following examples are provided to illustrate certain particular features and/or embodiments. The examples should not be construed to limit the disclosure to the particular features or embodiments described.

### Example 1

### Method for Generating a Seasonal, Broadly Reactive, Pan-Epitopic Influenza Virus HA Antigen Based on Drift Variants

The steps of the method of generating a non-naturally occurring, broadly reactive, immunogenic influenza HA antigen based on human flu seasons as described herein are summarized below. A general depiction of the method is shown in FIG. 1. Optimally, the results of some or all of the steps of the method may be displayed in a visual form on a display device.

A. Obtain antigen (e.g., HA) sequences from an online database (flu database), e.g., via the Global Initiative on Sharing All Influenza Data (“GISAID”) or the Influenza Research Database (“FluDB”), saving as FASTA amino acid sequences with accession ID information, and analyze HA (or NA) amino acid sequences from numerous virus strains (e.g., H1, H5, H7, etc.), (e.g., 6,430 HA amino acid sequences) over a given time period (e.g., 1968-2013). For example, such a search query can include “Northern Hemisphere: 10/1/xx (month/date/year)-4/30/xx+1 (month/date/year+1);” “Southern Hemisphere: 5/1/xx-9/31/xx;” “Yearly: 1/1/xx-12/31/xx.”

B. Assemble the multiple sequences and perform alignments for the sequences over the demarcated time period and geographical location using MUSCLE (MUltiple Sequence Comparison by LogExpectation) alignment (Clustering NJ, Sequence weighting scheme CLUSTALW), which is faster than Clustal and provides accurate alignments.

C. Eliminate incomplete HA sequences (retain HAI (HA head region sequences), amino acids 20-300 of the complete HA amino acid sequence) and remove partial sequences, for example, sequences of less than 280 residues, or sequences with gaps or ambiguities.

D. Assemble a phylogenetic tree by performing sequence alignments, based on the parameters: Jukes-Cantor genetic distance model, Neighbor joining phylogenetic tree building method, and no outgroup. For the phylogenetic tree, select the farthest upstream node to each defined clade or branch. While “node” and “cluster” are selected, color the node with a unique color. The parameters, phylogenetic trees and/or sequences therein are optimally displayed in a visual form on a display device.

E. Transform branches to “cladogram,” max characters “30,” and “Enable all layouts for unrooted trees.” The cladogram can be displayed in a visual form on a display device.

F. Select branches comprising multiple aligned sequences from those phylogenetic trees that have >98% sequence identity and demarcate the tree branches with color/cluster ID. Note whether substitution per site rate distance is >0.001. Clustering parameters are revised to have more stringent criteria, i.e., sequence similarity within the antigenic regions or specific antigenic sites.

G. Extract cluster(s) with substitution per site of >0.001 and use the cluster(s) to generate a primary influenza virus sequence of sequence similarity and/or identity for building future secondary sequences having sequence similarity and/or identity. Repeat steps F and G until primary influenza virus sequences have been generated for an entire tree from step E.

H. Generate an alignment and phylogenetic tree of all influenza virus primary sequences from step G (repeat as necessary). The radial phylogenetic tree generated to identify clusters of influenza virus antigen (e.g., HA) sequences or clusters of influenza virus HA sequences in a given geographic location (e.g., Southern Hemisphere) in a season (e.g., 2002), and/or selected branches of sequences from the phylogenetic tree can be displayed in a visual form on a display device.

I. Designate clusters of +5 primary sequences and extract sequences of similarly and/or identity (redefine/color) as a secondary cluster;

J. Generate a phylogenetic tree of influenza virus secondary sequences of similarity and/or identity from step I and assess whether multiple branches with substitution per site of >0.001 to assess variability among the sequences;

K. Combine multiple branches if the substitution rate per site distance is <0.001 and generate a sequence having sequences of similarity and/or identity from the cluster to produce an influenza virus tertiary sequence; L. Repeat for each time period of primary sequences, i.e., Scenario 1: 2009-2012, 2009-2013 (or 2010-2013), 2009-2014 (or 2011-2014), or Scenario 2: 2009-2012, 2010-2013 (or 2009-2013), 2011-2014 (or 2009-2014).

FIGS. 2A-2D depict aspects of the described method.

The method further includes translating the output of one or more of the various steps of the method into a visual form such as displaying the output on a display device.

### Example 2

### Construction and Synthesis of Pan-Epitopic HA Antigens for Use as Immunogens to Elicit Broadly Reactive Antibodies

This Example describes the generation of broadly reactive, pan-epitopic HA antigen sequences using the method as described herein. In general, a backbone sequence representing HA amino acid sequences from a 2002-2005 time period was generated by the method, followed by the “rolling” method as described herein to arrive at a broadly reactive, pan-epitopic sequence that captures HA epitopes from past and present seasons, e.g., 2005-2007, in this Example.

For H5 virus, influenza HA protein sequences from 2,456 H5Nx infections isolated from Jan. 1, 2011-Dec. 31, 2013 were downloaded from the Global Initiative on Sharing Avian Influenza Data (GISAID) database by date of isolation and categorized by each calendar year. For each round of sequence similarity/identity generation, multiple sequence alignment was performed using the GENEIOUS® MUSCLE alignment method, and Jukes-Cantor phylogenetic neighbor-joining, no out-grouping, unrooted circular trees (sequence clusters) were constructed.

HA sequences representing amino acids 17-338 of the HAI head region were aligned for each cluster, and the most common residue found among a designated set of virus HA sequences was used to yield the primary sequence. Ambiguities were avoided through alignment of ≥3 sequences. Partial sequences were excluded from the analysis.

Multiple rounds of primary sequence assembly from within a year were layered together to yield secondary sequences for that specific year. This methodology was used for each year during the time period (Jan. 1, 2011-Dec. 31, 2013), which yielded 40 sequences. These secondary HAI sequences were aligned in a GENEIOUS® MUSCLE alignment method, and Jukes-Cantor phylogenetic neighbor-joining, no out-grouping, unrooted linear trees were constructed. A five tertiary (3°) HAI sequence was determined and was termed the backbone sequence comprising the 2011 to 2013 time period. A flowchart of the method is shown in FIG. 1.

Additional next generation HAI head region sequences (i.e., HAI sequences expected to reflect a composite of epitopes (antigenic determinants)) from viral antigen sequences representing viruses from different seasons and from different geographic locations) were developed by one of three scenarios (Scenario #1, #2, or #3). 40 HA sequences isolated during the years (Jan. 1, 2014-Dec. 31, 2014) were downloaded. The same methodology that resulted in an unrooted circular tree alignment with 12 primary clusters, which, when layered together, produced 12 new, secondary HA sequences from this year.

In Scenario #1, these 12 new, secondary sequences were added to the previously generated secondary backbone linear phylogenetic alignment, and the 12 oldest secondary sequences that were produced from the 2011 year (Jan. 1, 2011-Dec. 31, 2011) were eliminated from the alignment. All 40 secondary HA sequences from each year were weighted equally and resulted in a 4 tertiary HA sequence. (FIGS. 2A-2C).

For Scenario #3, the same 12 new, secondary sequences were added to the previously generated secondary backbone linear phylogenetic alignment, but unlike Scenario #1, none of the older secondary sequences (Jan. 1, 2011-Dec. 31, 2011) were eliminated from the alignment; instead, the older secondary sequences extended the era included to capture an additional season, and increased the number of secondary sequences used to generate the final tertiary HA sequence to 3. (FIGS. 2A and 2D).

Both Scenarios were used to include sequences from influenza season through the 2014. Scenario #1 produced 4 HA sequences, 3 of which was unique, and Scenario #3 produced 3 HA sequences; 2 of these tertiary HA sequences was unique. In the end, the process resulted in the generation of a total of 5 new tertiary HA sequences that were produced.

These tertiary sequences were aligned with the wild-type vaccine stains, as well as HA sequences from co-circulating variants, in a final phylogenetic tree. The 5 H5Nx HA constructs were synthesized and inserted into the pTR600 expression vector, as previously described and referenced supra. Examples of H5 HA sequences (571 amino acids), generated using described method are presented in FIG. 3.

To generate the HA2 stalk region, sequences were designed for the 2011-2017 time frame, the leader sequence (AA 538-571) from various viruses was used in combination with the final broadly reactive antigen HAI and HA2 antigen protein sequences to generate a full length sequence (571 amino acids (AA); 1,617 nucleotides). For the leader sequence, (AA 1-16) were derived from A/Chicken/Ghana/15BIR5480-27/2015 influenza virus and was used in combination with the final broadly reactive antigen HAI and HA2 antigen protein sequences to generate a full length (571 AA sequence).

### Example 3

### Hemagglutination-Inhibition (HAI) Assay

The hemagglutination inhibition (HAI) assay was used to assess functional antibodies to the HA protein that are able to inhibit agglutination of guinea pig, horse, or turkey erythrocytes (red blood cells (RBCs)). The protocols were adapted from the WHO laboratory influenza surveillance manual (Gillim-Ross and Subbarao, 2006, Clin Microbiol Rev 19(4):614-636) and use the host-species that is frequently used to characterize contemporary H3N2 strains that have preferential binding to alpha (2, 6) linked sialic acid receptors. Turkey or guinea pig erythrocytes were used to compare whether there was a difference in HAI depending on the type of erythrocyte that was used.

To inactivate nonspecific inhibitors, sera were treated with receptor-destroying enzyme (RDE) (Denka Seiken, Co., Japan) prior to being tested. (Bright et al., 2005, Lancet 366(9492):1175-1181; Bright et al., 2003, Virology 308(2):270-278; Bright et al., 2006, JAMA 295(8):891-894; Mitchell et al., 2004, Vaccine 21(9-10):902-914; Ross et al., 2000, Nat Immunol 1(2):127-131). Briefly, three parts of RDE was added to one part of sera and incubated overnight at 37° C. RDE was inactivated by incubation at 56° C. for approximately 30 minutes (˜30 min.). RDE-treated sera were diluted in a series of two-fold serial dilutions in v-bottom microtiter (multi-well) plates. An equal volume of each virus, e.g., H3N2 virus, adjusted to approximately 8 hemagglutination units (HAU)/50 μl, was added to each well. The plates were covered and incubated at room temperature for 20 minutes, followed by the addition of 0.8% guinea pig erythrocytes (Lampire Biologicals, Pipersville, Pa., USA) in phosphate buffered saline (PBS). Red blood cells were stored at 4° C. and used within 72 hours of preparation.

The plates were mixed by agitation and covered, and the RBCs were allowed to settle for 1 hour at room temperature. The HAI titer was determined by the reciprocal dilution of the last well that contained non-agglutinated RBCs. Positive and negative serum controls were included for each plate. All mice were negative (HAI ≤1:10) for preexisting antibodies to currently circulating human influenza viruses prior to vaccination. Seroprotection was defined as HAI titer >1:40, and seroconversion was defined as a 4-fold increase in titer compared to baseline, as per the WHO and European Committee for Medicinal Products to evaluate influenza vaccines. A more stringent threshold of >1:80 was often examined. Because mice are naïve and seronegative at the time of vaccination, seroconversion and seroprotection rates are interchangeable in the experiments.

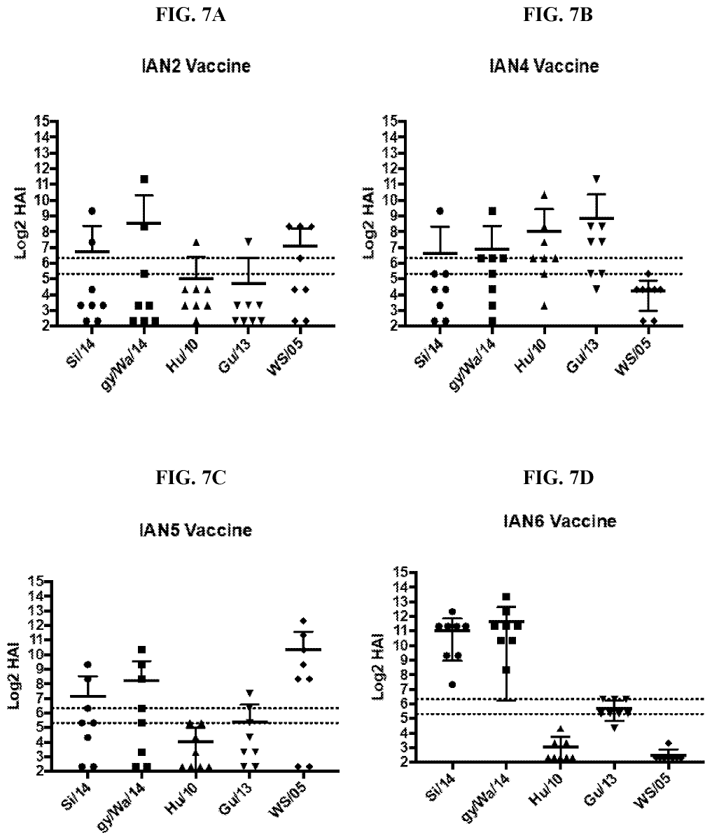

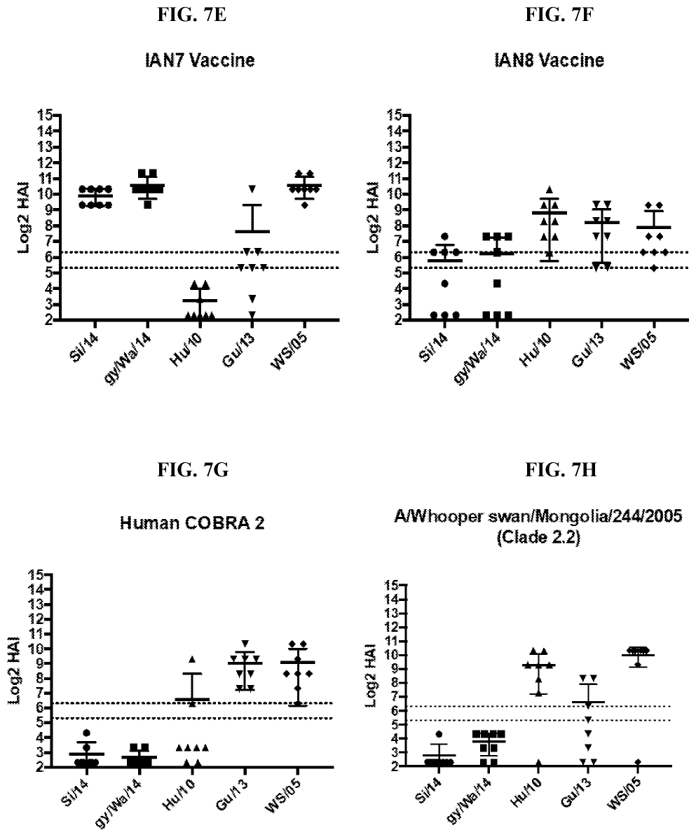

Results of HAI assays using sera from animals immunized with virus derived H5 HA immunogens (VLPs), i.e., IAN2, IAN4, IAN5, IAN6, IAN7 and IAN8, versus controls, are shown in FIGS. 7A-71I, as described supra. Results of HAI assays using sera from animals immunized with virus derived HA immunogens (VLPs) and H1 V9 HA immunogen (VLP), (FIG. 11) are shown in FIGS. 12A-12D.

### Example 4

### Enzyme-Linked Lectin Assay (ELLA)

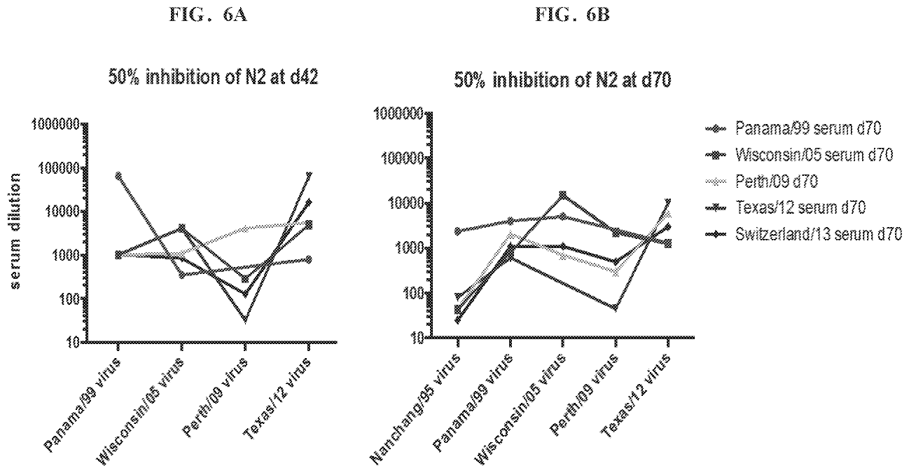

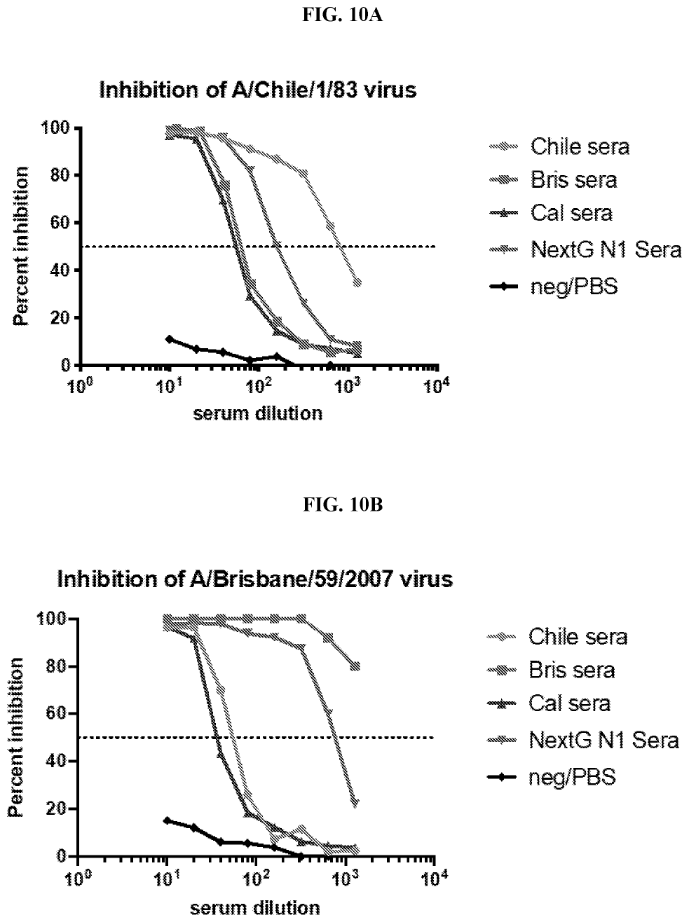

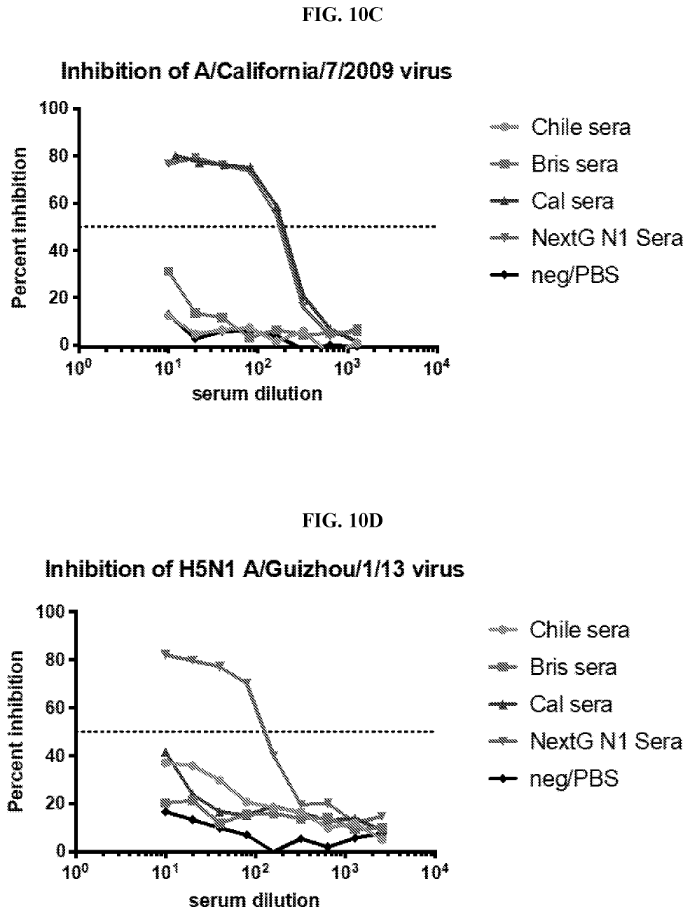

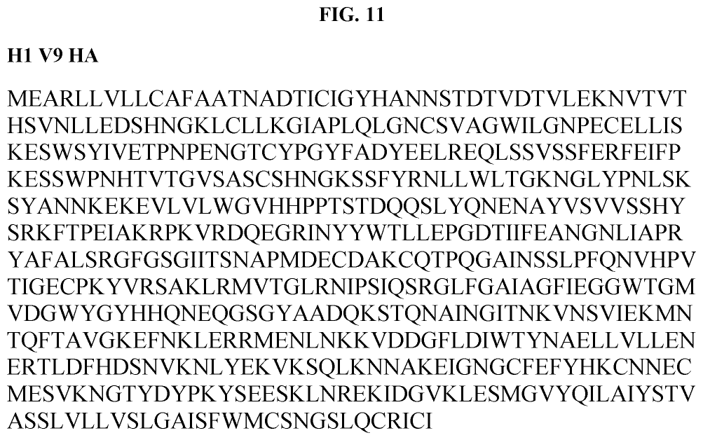

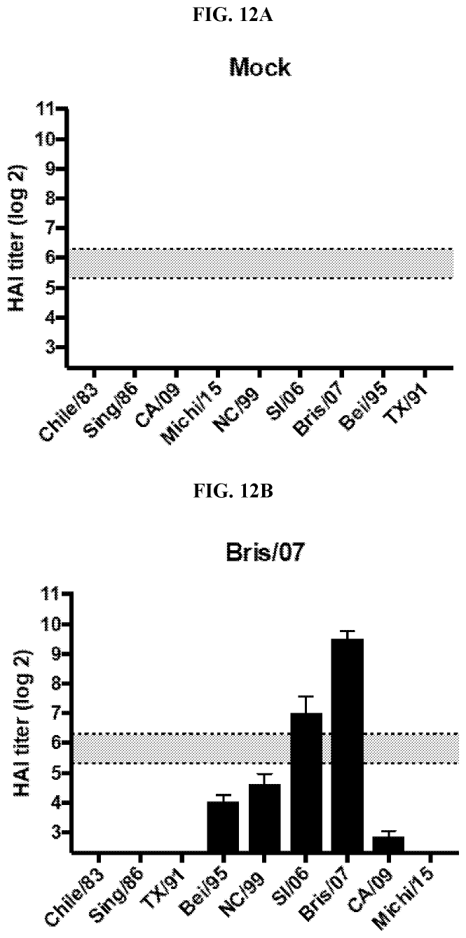

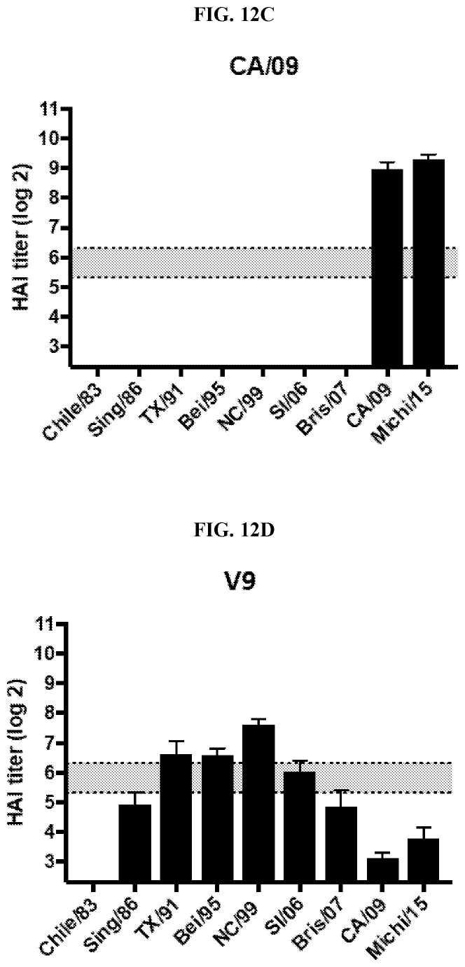

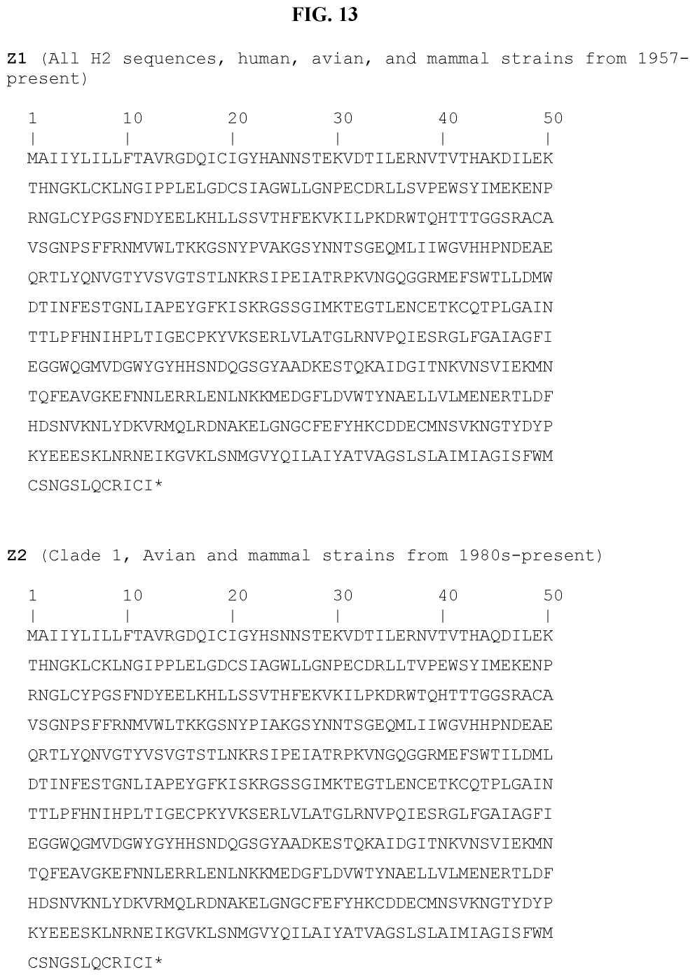

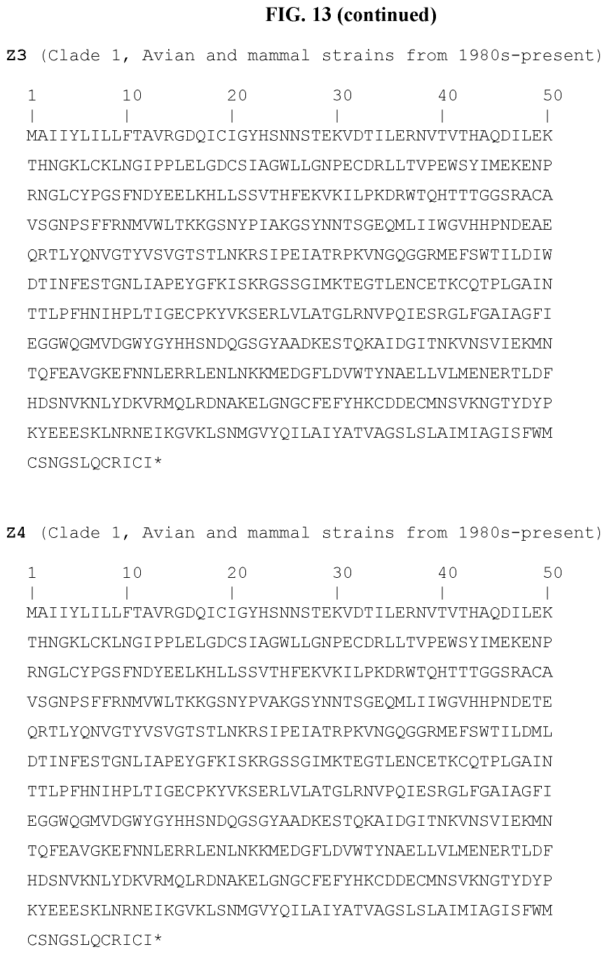

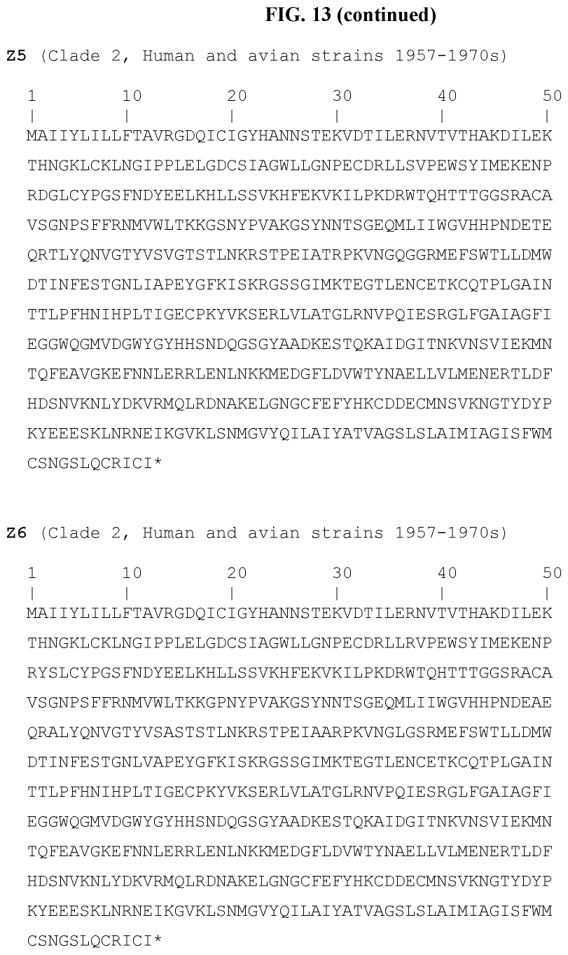

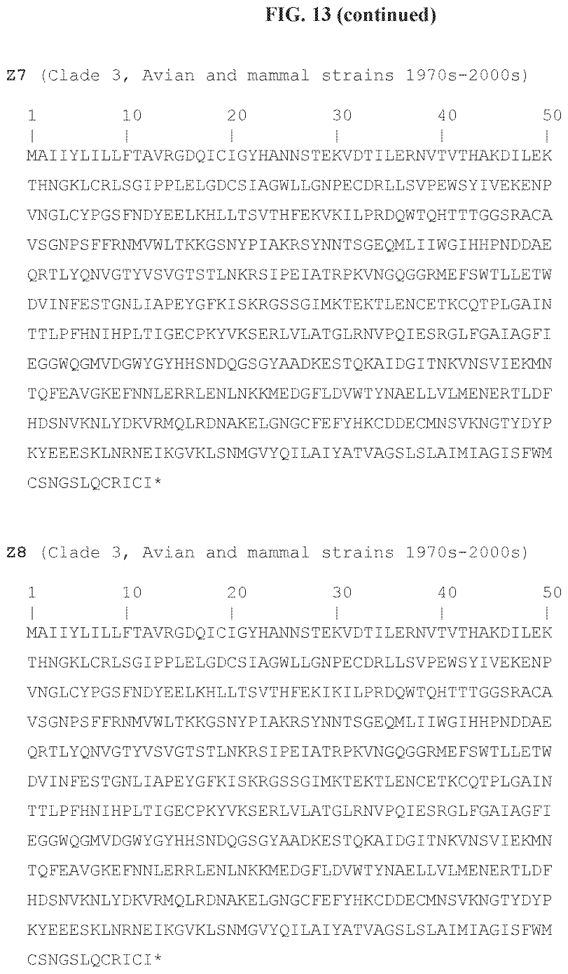

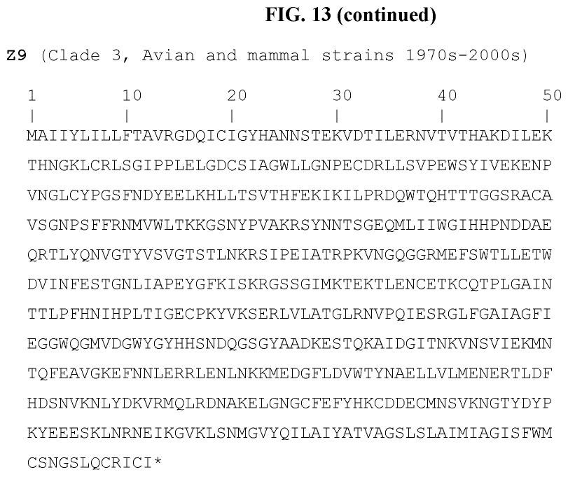

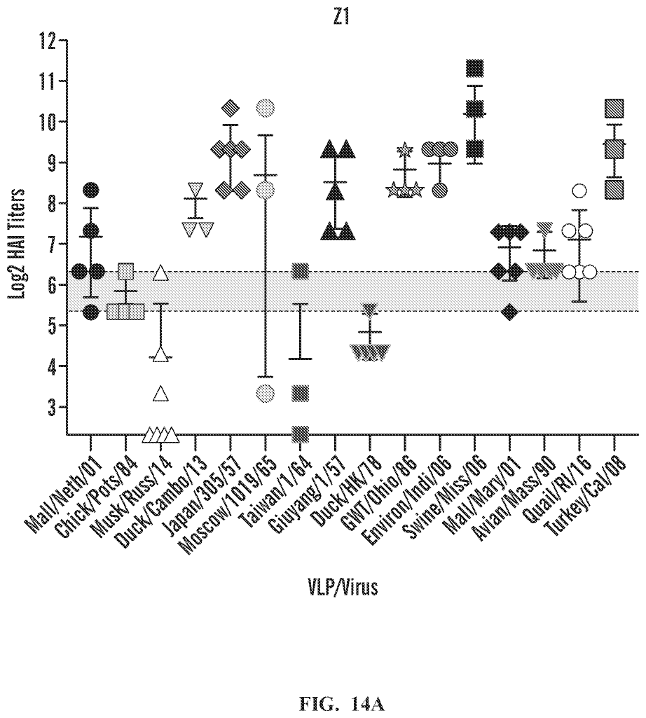

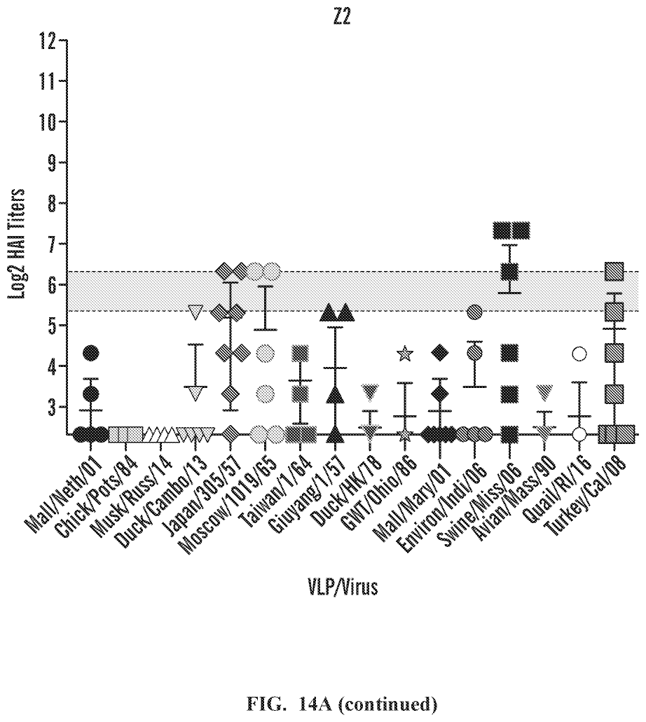

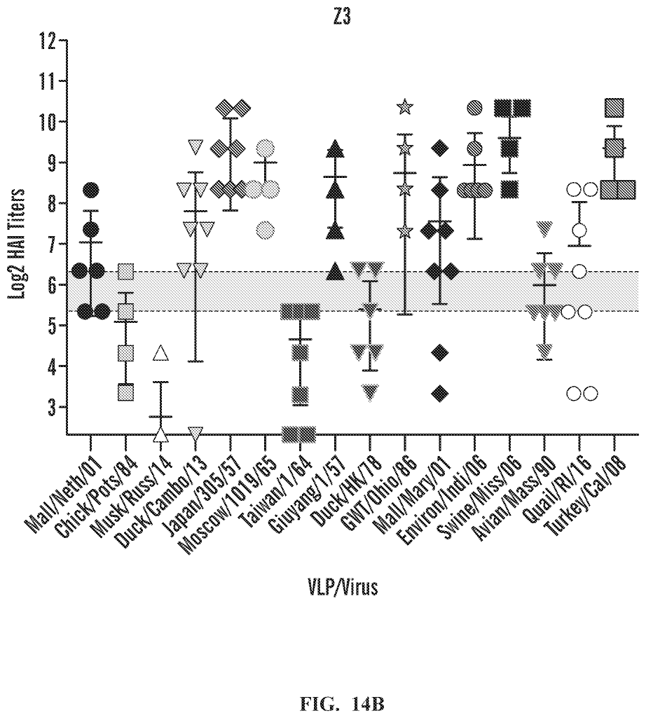

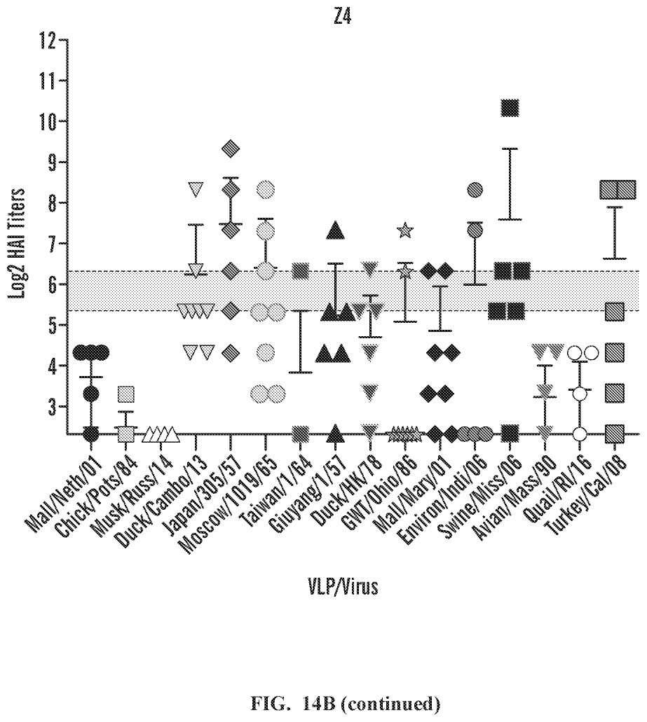

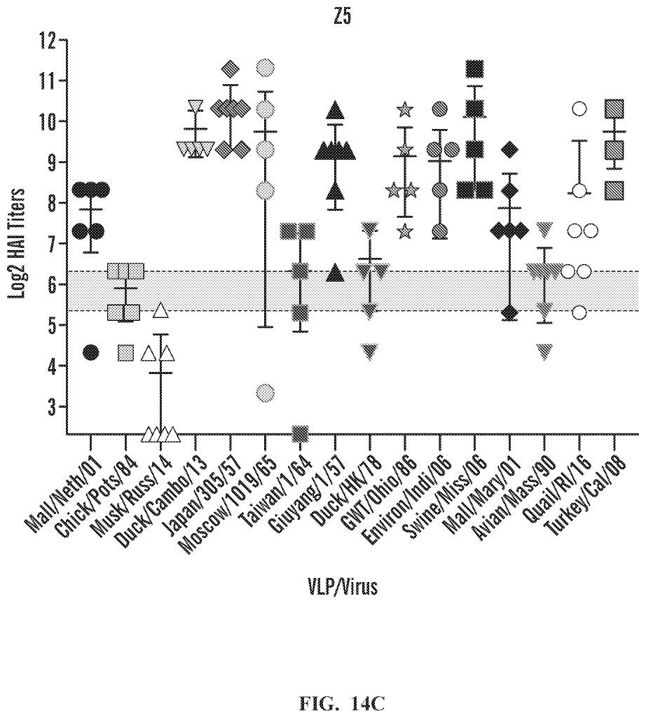

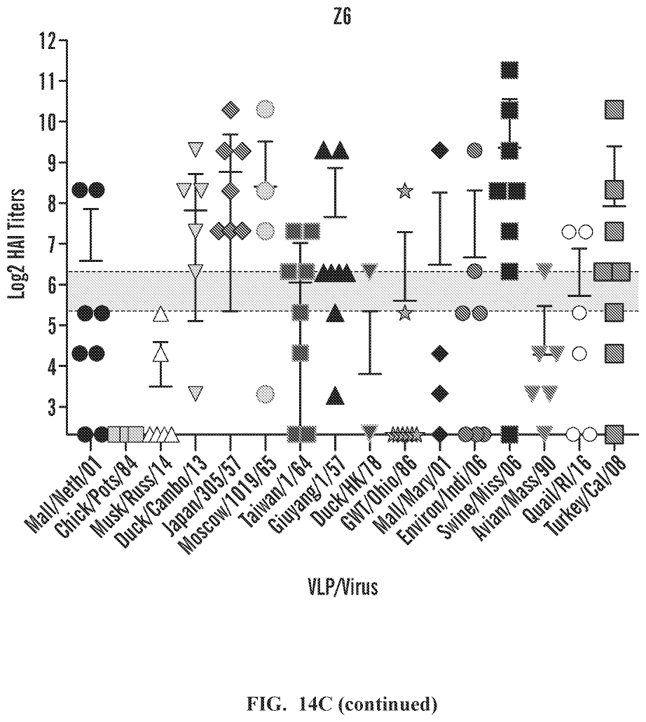

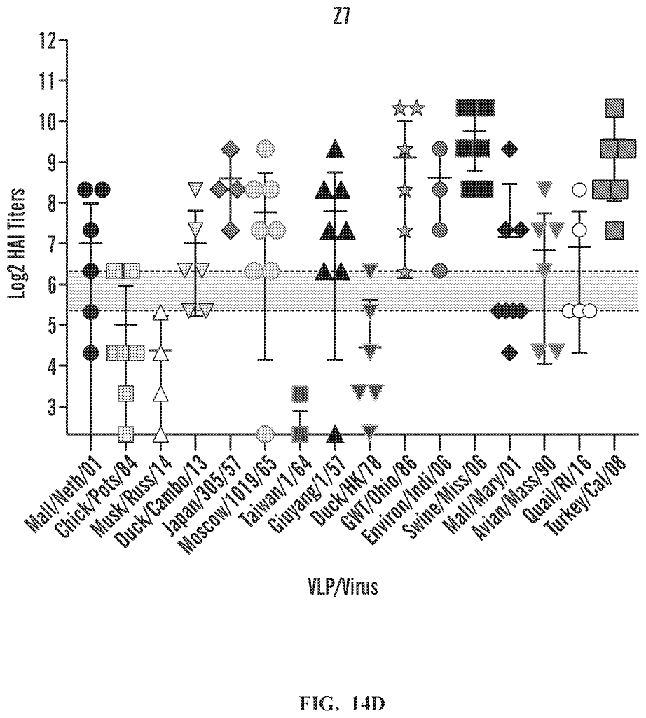

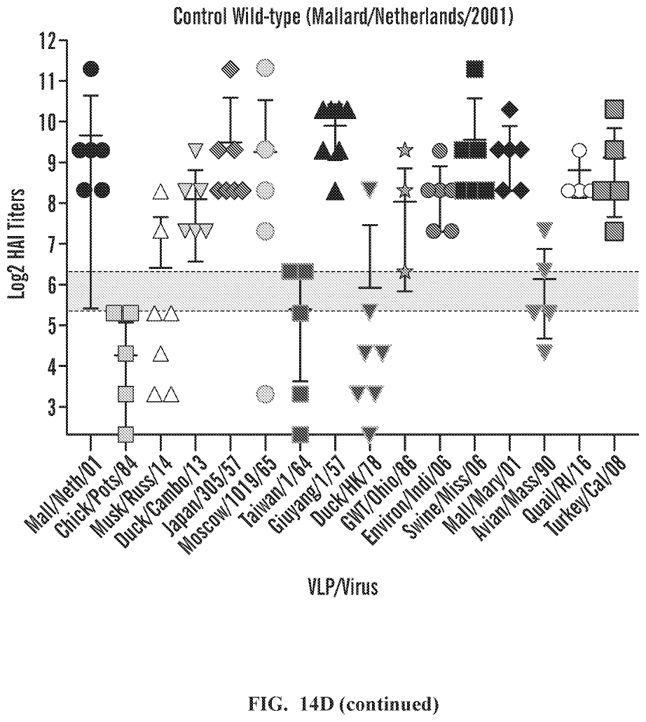

To determine the amount of neuraminidase (NA) inhibiting antibodies were present in a sample, such as in immunized animal sera, an enzyme-linked lectin assay (ELLA) was performed as described by L. Couzens et al., 2014. J. Virol. Methods, Vol. 210, pp. 7-14). Briefly, flat-bottom, Maxisorp polystyrene 96-well plates (Maxisorp, Nunc) were coated with fetuin (100 μL; Sigma-Aldrich) at 25 μg/ml at 4° C. overnight. Serum samples were heat-treated at 56° C. for 1 hour prior to serial two-fold dilutions in PBS and subsequent co-incubation with a predetermined 90% NA activity at 37° C. for 16-18 hours. After three wash steps with PBS containing 0.05% Tween-20 (PBS-T), peroxidase-labeled lectin from Arachis hypogaea (Sigma-Aldrich) was added and the samples were incubated for 2 hours at room temperature in the dark. Plates were washed again before adding o-phenylenediamine dihydrochloride (OPD) substrate (Sigma-Aldrich). The reaction was stopped with 1N sulfuric acid before reading the absorbance at 492 nm. The sialidase-inhibiting antibody titer was expressed as the reciprocal of the highest dilution that exhibited ≥50% inhibition of NA activity. Results of NA inhibition assays are shown in FIGS. 6A and 6B and FIGS. 10A-10D.

### Example 5

### Virus-Like Particle (Vaccine) Preparation

Mammalian 293T cells were transfected with each of three mammalian expression plasmids expressing either the influenza neuraminidase (A/mallard/Alberta/24/01, H7N3), the HIV p55 Gag sequences, or one of the broadly reactive, ‘Next Generation’ HA expression plasmids (e.g., containing a polynucleotide sequence encoding an HA, HA1, HA2, or NA protein, for example, as shown in FIG. 3 and FIG. 4), using methods practiced by those having skill in the art (see, e.g., U.S. Patent Application Publication US 2015/0030628). Following 72 hours of incubation at 37° C., supernatants from transiently transfected cells were collected, centrifuged to remove cellular debris, and filtered through a 0.22 μm pore membrane. Mammalian virus-like particles (VLPs) were purified and sedimented by ultracentrifugation on a 20% glycerol cushion at 135,000×g for 4 hours at 4° C. VLPs were resuspended in phosphate buffered saline (PBS) and total protein concentration was assessed using a conventional bicinchoninic acid assay (BCA). The hemagglutination activity of each preparation of VLPs was determined by adding an equal volume of turkey red blood cells (RBCs) to a V-bottom 96-well plate and incubating with serially-diluted volumes of VLPs for 30 minutes at room temperature (RT). The highest dilution of VLP with full agglutination of RBCs was considered the endpoint HA titer.

### Example 6

### Determination of HA Content by Enzyme Linked Immunosorbent Assay (ELISA)

A high-affinity, 96-well, flat-bottom ELISA plate was coated with 5-10 μg of total protein of VLP and serial dilutions of a recombinant H3 antigen (3006_H3_Vc, Protein Sciences, Meriden, Conn.) in ELISA carbonate buffer (50 mM carbonate buffer, pH 9.5) were added to the wells. The plate was incubated overnight at 4° C. on a rocker. The next morning, the plates were washed in PBS with 0.05% Tween-20 (PBST), and non-specific epitopes were blocked with 1% bovine serum albumin (BSA) in PBST solution for 1 hour at RT. The buffer was removed, and stalk-specific Group 2 monoclonal antibody CR8020 (Tharakaraman, K. et al., 2014, Cell Host & Microbe, Vol. 15, pp. 644-651; Ekiert, D. C. et al., 2012, Science, 333(6044):843-850; Creative Biolabs, Shirley, N.Y.) was added to plate, followed by a 1 hour incubation at 37° C. The plates were washed and then were probed with goat anti-human IgG horseradish-peroxidase-conjugated secondary antibody (2040-05, Southern Biotech, Birmingham, Ala.) for 1 hour at 37° C.

The plates were washed. Freshly prepared o-phenylenediamine dihydrochloride (OPD) (P8287, Sigma, City, State, USA) substrate in citrate buffer (P4922, Sigma) was then added to wells, followed by the addition of 1N H2504 stopping reagent. The plates were read at 492 nm absorbance using a microplate reader (Powerwave XS, Biotek, Winooski, Vt.). Background signal was subtracted from negative wells. Linear regression standard curve analysis was performed using the known concentrations of recombinant standard antigen to estimate the HA content in lots of VLPs.

### Example 7

Generation of 112 HA Immunogens that Elicit Broadly Reactive Antisera in Immunized Mice

The hemagglutination inhibition (HAI) assay described above (Example 3) was used to assess functional antibodies to the HA protein that were generated in mice following immunization with VLPs expressing each of the H2N1 HA antigens Z1-Z7 (FIG. 13). The hemagglutinin inhibition activity of antibodies in the antisera of mice following immunization is demonstrated in FIGS. 14A-14D. BALB/c mice (8 mice per immunogen tested) were immunized with VLPs expressing the H2N1 HA antigens Z1-Z7, as shown above each of the graphs in FIGS. 14A-14D. The sera from each mouse were collected and assessed in the HAI assay. All virus strains tested against the sera in the HAI assay are presented on the x-axes. The horizontal gray bar indicates an antiserum titer of 1:40-1:80. A titer of 1:40 is considered to provide the minimal protection in humans for seasonal influenza. The HAI assay results indicate that, following immunization, the Z1, Z3, Z5, and Z7 H2 HA protein immunogens generated broadly reactive antisera that effectively neutralized HA from a variety of virus strains as well as, or better than, antisera from animals immunized with control wild-type virus (Mallard/Netherlands/2001), which typically produces high titer antisera in animals following immunization.

### Example 8

Immunization with Neuraminidase (NA) Immunogen Provides Survival of Animals Following Virus Challenge

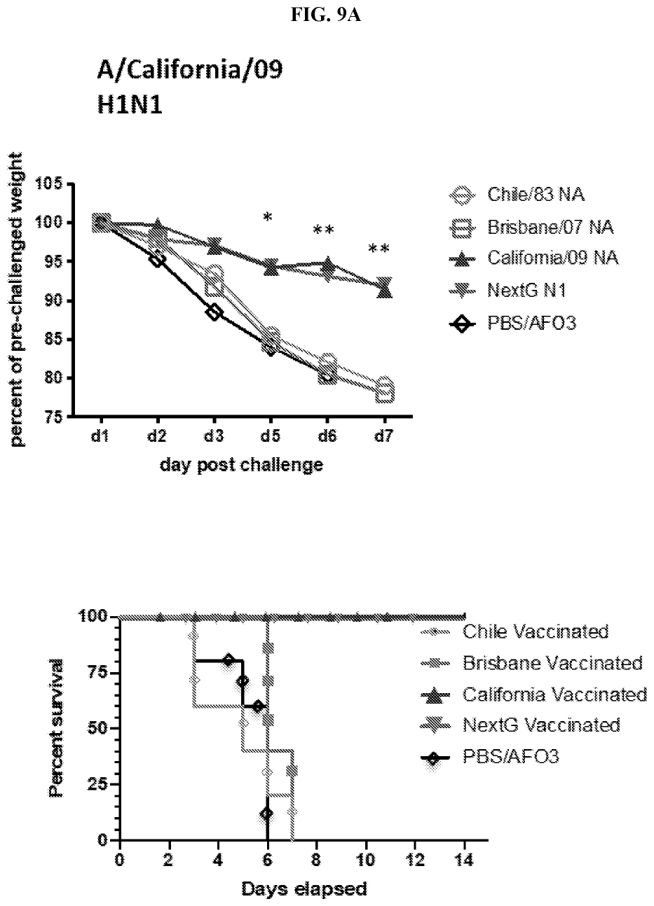

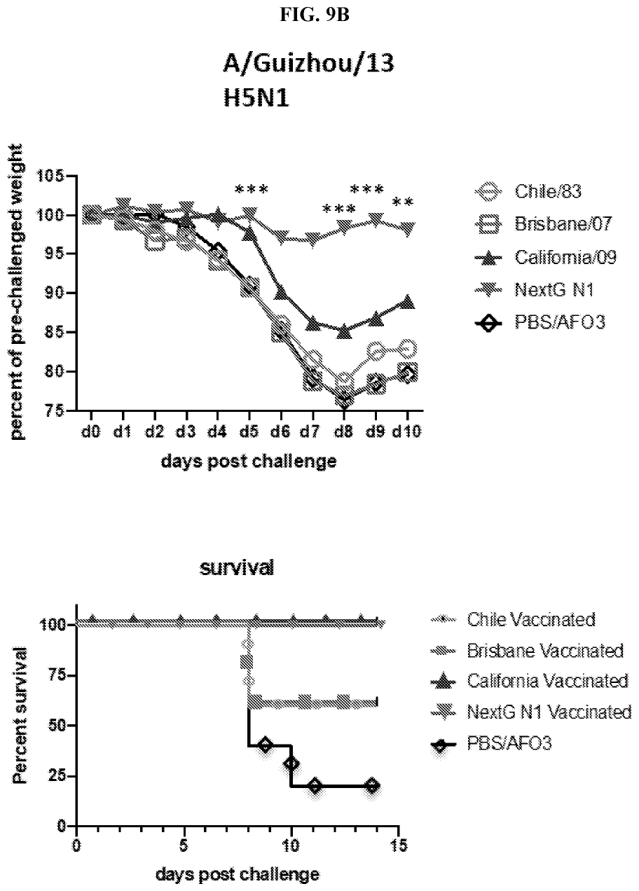

FIGS. 9A and 9B illustrate the results of challenge and survival of mice with different virus types after immunizing the animals with VLPs expressing a neuraminidase protein (N1) immunogen (NextGen N1). For the studies, groups of 10 mice were immunized in a prime-boost-boost regimen with wild-type N1 VLPs or with NextGen N1 VLPs and were challenged with either an H1N1 virus type (A/California/07/2009) or an H5N1 virus type (A/Guizhou/1/2013). Shown in FIG. 9A is the percent of animals' pre-challenged weight (top graph) and survival (bottom graph) after challenge with H1N1 virus A/California/07/2009. Shown in FIG. 9B is the percent of animals' pre-challenged weight (top graph) and survival (bottom graph) after challenge with H5N1 virus A/Guizhou/1/2013. Animal weight and scores were recorded daily.

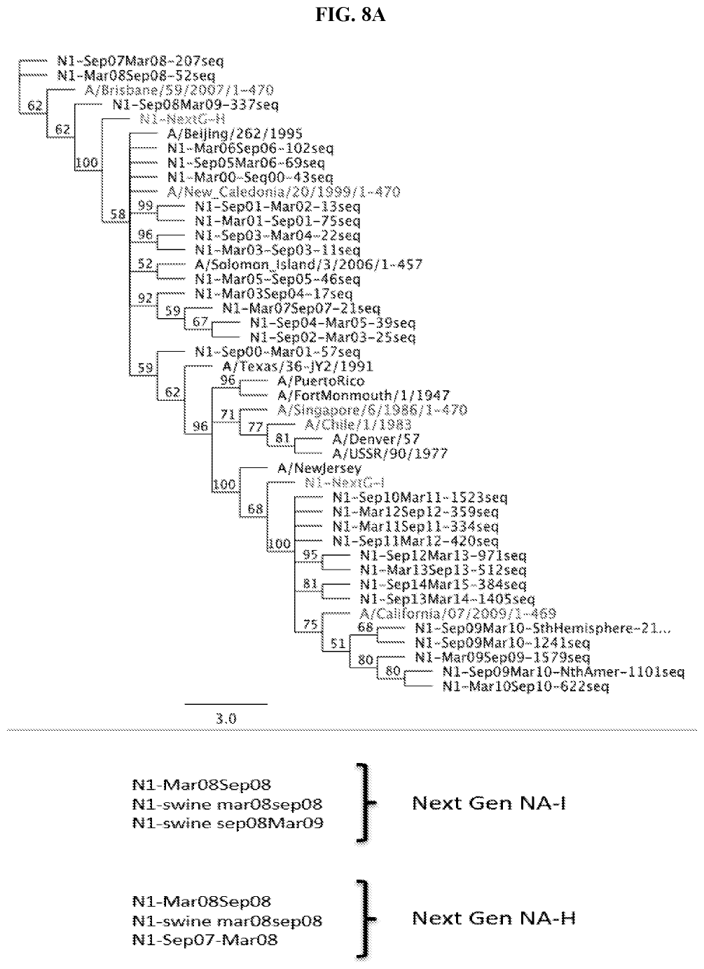

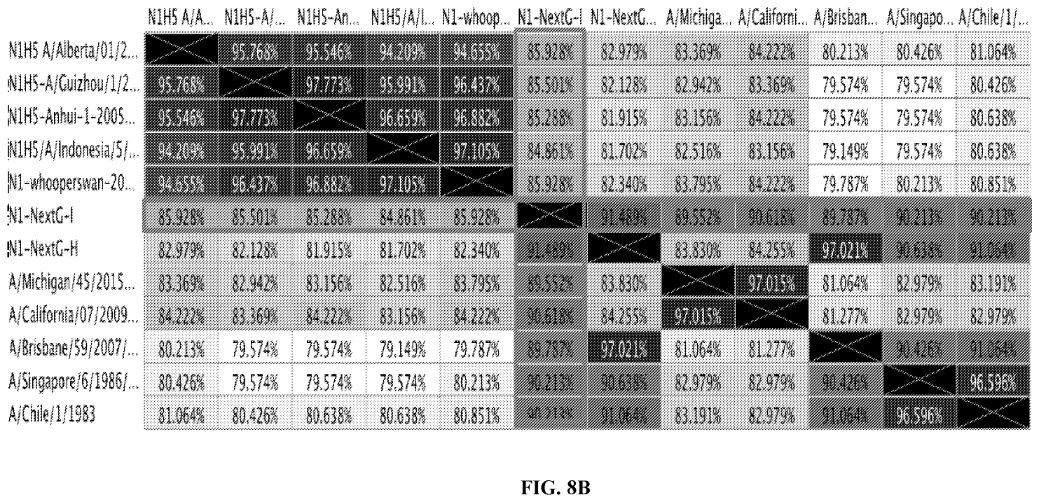

The neuraminidase (N1) protein antigen used as immunogen in the studies was designed to account for the evolution of influenza virus neuraminidase protein (NA) sequences over time using the methods as described herein. Full-length influenza A neuraminidase (NA) amino acid sequences isolated from human ENNI (e.g., HINI) infections from the GISAID (Global Initiative on Sharing All Influenza Data) Influenza Virus platform from 2000 to 2013 were used in accordance with the methods described herein to generate and identify primary sequences of sequence similarity and/or identity separated by hemisphere and assessed by influenza season with Geneious Muscle software (BioMatters Ltd). Secondary sequences were created by assessment of human and swine primary sequences having similarity and/or identity over several seasons. (FIGS. 8A and 8B). The resulting NextGen NA sequences were synthesized and inserted into the pTR600 expression vector as described by Ross et al., 2000, Nat Immunol. 1(2):102-103).

### Example 9

### Mouse and Ferret Studies

### Mouse Studies

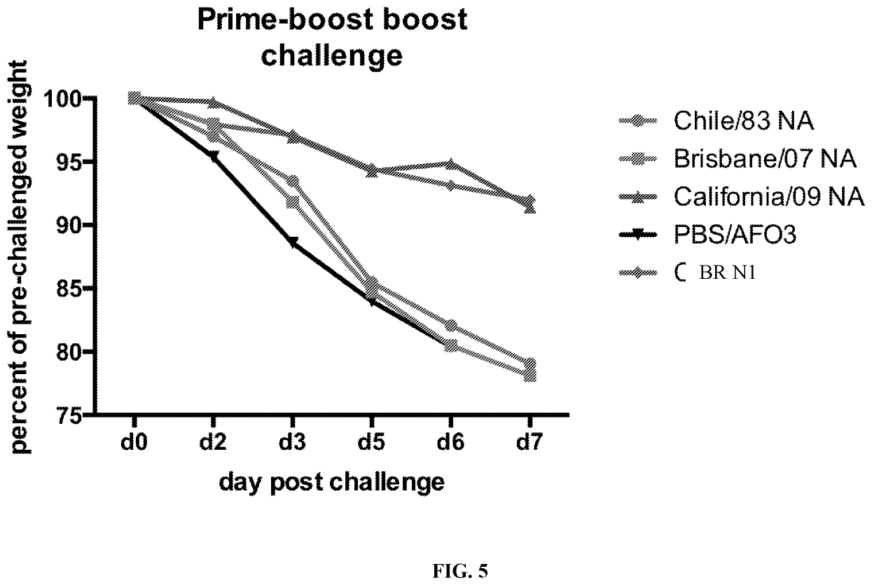

BALB/c mice (Mus musculus, females, 6 to 8 weeks old) were purchased from Jackson Laboratory (Bar Harbor, Me., USA), housed in microisolator units and allowed free access to food and water. The animals were cared for under University of Georgia Research Animal Resources guidelines for laboratory animals. All procedures were reviewed and approved by the Institutional Animal Care and Use Committee (IACUC). Mice (5 or 10 mice per group) were administered (vaccinated with) purified virus-like particles (VLPs), (3.0 μg/mouse), based upon HA content from the ELISA quantification, and VLP vaccines were delivered to the animals via intramuscular injection at week 0. A prime-boost boost regimen for VLP administration was used. Animals were boosted with the same vaccine at the same dose at weeks 4 and 8. Vaccines at each dose were formulated with an emulsified squalene-in-water adjuvant (Sanofi Pasteur, Lyon, France). The final concentration after mixing 1:1 with VLPs is 2.5% squalene. Twenty-eight days after each vaccination, blood samples were collected via the submandibular cheek, and the samples were transferred to a microcentrifuge tube. The tubes were centrifuged at 10,000 rpm for 10 minutes. Serum samples were removed and frozen at −20° C.±5° C. Results of a prime-boost challenge using different NA immunogens as VLPs administered to mice, including a broadly reactive NA N1 immunogen (BR N1) generated by the described methods, are shown in FIG. 5.

### Ferret Studies

Fitch ferrets (Mustela putorius faro, female, 6-12-months of age), influenza naive and de-scented, were purchased from Marshall Farms (Sayre, Pa., USA). Ferrets were pair-housed in stainless steel cages (Shor-line, Kansas City, Kans., USA) containing Sani-chips Laboratory Animal Bedding (P.J. Murphy Forest Products, Montville, N.J., USA). Ferrets were provided with Teklad Global Ferret Diet (Harlan Teklad, Madison, Wis., USA) and fresh water ad libitum.

The purified VLPs were diluted in PBS, pH 7.2, to achieve final concentration. Ferrets (n=3) were vaccinated with 15 μg of purified VLPs, based upon HA content as determined by densitometry assay, via intramuscular injection in the quadriceps muscle in a volume of 0.25 ml at week 0, and then were boosted with the same dose at week 3. Vaccines were stored at −80° C. prior to use and formulated with IMJECT® alum adjuvant (IMJECT® Alum; Pierce Biotechnology, Rockford, Ill. USA) or with the above-described emulsified squalene-in-water adjuvant immediately prior to use. Animals were monitored for adverse events including weight loss, temperature, loss of activity, nasal discharge, sneezing and diarrhea weekly during the vaccination regimen. Prior to vaccination, animals were confirmed by HAI assay to be seronegative for circulating influenza A (e.g., H1N1) and influenza B viruses. Fourteen to twenty-one days after each vaccination, blood was collected from anesthetized ferrets via the anterior vena cava and transferred to a microfuge tube. The tubes were centrifuged; serum was removed and frozen at −20±5° C.

### Example 10

### Next-Generation Methodology to Improve Upon and Advance the Generation of Existing Pathogen-Derived Antigen Sequences and Produce Seasonal, Broadly Reactive, Pan-Epitopic H3 HA Antigen Based on Drift Variants

The next-generation methodology described in this Example was developed to improve upon and advance the generation of existing pathogen-derived antigen sequences, (e.g., influenza virus HA), produced by prior computationally optimized methods. The next-generation methodology involved the generation of pathogen-derived antigen sequences to produce immunogenic sequences in a real-time fashion at selected time periods, e.g., every six months; so as to include new antigen sequences as new pathogenic strains emerged in the wild; and to incorporate future antigen sequences of emerging pathogens (e.g., influenza viruses) into a final pan-epitopic antigen sequence for use as an immunogen. The method described below represents a next-generation technology for generating broadly reactive antigen sequences from the HAI portion of influenza virus for use as immunogens.

In the method, a backbone sequence was created by downloading sequences from public databases, e.g., GISAID or FluDB, within a given time period, e.g., a six-month period, for a given subtype, e.g., H3N2. Following alignment of the sequences, the HAI portion (amino acids 20-300) was typically extracted, and a phylogenetic tree was created. The tree was divided into different primary clusters. Each cluster contained sequences that were at least about 98% identical to each other, and a primary sequence was generated from each cluster. This process was repeated until the entire phylogenetic tree was divided into multiple primary sequences, which were then realigned to one another. A new phylogenetic tree was created using only the primary sequences. Three or more primary sequences that were located in close proximity to each other in the tree were then extracted, aligned, and a new secondary sequence was formed. These steps were repeated until all of the primary sequences were utilized to generate multiple secondary sequences.

The above-described steps were repeated for the following six-month period until a series of five consecutive, 6-month periods were captured. All of the secondary sequences from a given 2.5-year period were then aligned; a phylogenetic tree was created, and the sequences that were close to each other in identity were used to generate multiple tertiary sequences. The tertiary sequences were aligned, and all of the tertiary sequences were used to generate a final quaternary backbone sequence.

Once a backbone sequence was generated for the antigen, new, emerging sequences were added in an effort to “roll” the design forward in time. By way of example, two different procedures were used to roll forward a new antigen design. In Scenario 1 (FIG. 2A), secondary sequences were added in from the most recent six-month period, and secondary sequences were ‘dropped off’ from the oldest six-month period used to construct the backbone sequence. After the first six months of rolling, the resulting antigen sequence was comprised of multiple secondary sequences that encompassed the most recent 2.5-year period. In Scenario 2 (and 3) (FIG. 2A), secondary sequences were added in from the most recent six-month period, and all of the previous secondary sequences from the generated backbone sequence were retained. After the first six months of rolling, the resulting antigen sequence was comprised of multiple secondary sequences that covered the most recent three-year period.

Similarly, an antigenic immunogen sequence for the HA2 portion of hemagglutinin (HA2), (amino acids 301-566) during the same time frame was generated using the above approach. Sequences were obtained from a six-month time frame; primary sequences were generated using the at least about 98% sequence identity criterion described above; similar primary sequences were grouped together to produce secondary sequences; and the secondary sequences were used to generate tertiary sequences. Lastly, all of the tertiary sequences that spanned 2.5 years of HA sequence coverage were aligned and a quaternary (backbone) HA antigenic immunogen was generated. The quaternary HAI and HA2 sequences were combined and coupled to a leader sequence (typically NH-terminal amino acids 1-19) that was derived from a wild-type strain of influenza virus to form the amino acid sequence of the pan-epitopic, broadly reactive antigenic immunogen.

### Other Embodiments

From the foregoing description, it will be apparent that variations and modifications may be made to the invention described herein to adopt it to various usages and conditions.

Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of generating a non-naturally occurring, pan-epitopic immunogen capable of generating an immune response in a subject, the method comprising:
(a) generating a phylogenetic tree comprising full length related antigen sequences derived from one or more pathogens or pathogen strains;
(b) identifying clusters of antigen sequences within the tree, each cluster having at least 95% identity and at least about 0.001 substitution per site relative to the other sequences within the cluster;
(c) generating for each cluster a non-naturally occurring primary sequence comprising amino acids that are conserved or identical within the cluster;
(d) generating a phylogenetic tree comprising the sequences of step (c);
(e) selecting three or more clusters of antigen sequences within the primary sequences, each selected cluster comprising at least about 0.001 amino acid substitutions per site and generating secondary sequences comprising amino acids that are conserved or identical within the three or more clusters;
(f) generating a phylogenetic tree comprising the secondary sequences, wherein branches of the tree are combined if the substitution rate per amino acid site distance is less than about 0.001 to produce a plurality of tertiary sequences;
(g) generating a quaternary backbone sequence comprising amino acids that are conserved or identical among the tertiary sequences; and
(h) generating a non-naturally occurring, pan-epitopic immunogen by incorporating secondary sequences from step (e), wherein the selected sequences are derived from the most recent time period.

2. The method of claim 1, wherein each pathogenic strain is present in two or more selected geographic regions over one or more selected periods of time.

3. A method of generating a non-naturally occurring pan-epitopic immunogen capable of generating an immune response against present and future influenza virus strains in a subject, the method comprising:
(a) generating a phylogenetic tree comprising full length related antigen sequences derived from one or more influenza virus strains, wherein each influenza virus strain is present in two or more selected geographic regions over one or more flu seasons;
(b) identifying clusters of antigen sequences within the tree, each cluster having at least 95% identity and at least about 0.001 substitution per site relative to the other sequences within the cluster;
(c) generating for each cluster a non-naturally occurring primary sequence comprising amino acids that are conserved or identical within the cluster;
(d) generating a phylogenetic tree comprising the primary sequences of step (c);
(e) selecting three or more clusters of antigen sequences within the primary sequences, each selected cluster comprising at least about 0.001 amino acid substitutions per site and generating secondary sequences comprising amino acids that are conserved or identical within the three or more clusters;
(f) generating a phylogenetic tree comprising the secondary sequences, wherein branches of the tree are combined if the substitution rate per amino acid site distance is less than about 0.001 to produce a plurality of tertiary sequences;
(g) generating a quaternary backbone sequence comprising amino acids that are conserved or identical among the tertiary sequences; and
(h) generating a non-naturally occurring, pan-epitopic immunogen by incorporating secondary sequences from step (e), wherein the selected sequences are derived from the most recent time period.

4. The method of claim 3, wherein the influenza virus strain is an influenza H5 strain and wherein the full length related antigen sequences of step (a) are derived from two or more H5 clades, or two or more H5 species, present in two or more selected geographic regions over one or more flu seasons;
(b).

5. The method of claim 3, wherein the influenza virus strain is an influenza H1 or H2 strain and wherein the full length related antigen sequences of step (a) are derived from H1 or H2 strains present during two or more consecutive flu seasons.

6-8. (canceled)

9. The method of claim 1, wherein the phylogenetic tree comprising full length related antigen sequences derived from Dengue strains present in the Americas or Asia during a selected period of time.

10-16. (canceled)

17. The method of claim 1, wherein the immunogen generated following step (h) is expressed, synthesized, isolated and/or purified.

18. The method of claim 1, further comprising formulating the immunogen for administration to a subject.

19. The method of claim 1, further comprising administering to a subject in need thereof an effective amount of the immunogen or a composition thereof to elicit an immune response in the subject.

20-35. (canceled)

36. A non-naturally occurring immunogen generated using the method of claim 1;
comprising an amino acid sequence of a hemagglutinin (HA) antigen as set forth in FIG. 3;
comprising an amino acid sequence that is at least 95% identical to an amino acid sequence of a neuraminidase (NA) antigen as set forth in FIG. 4.

37-41. (canceled)

42. An immunogenic composition or vaccine comprising the immunogen of claim 36.

43. A virus-like particle (VLP) comprising the immunogen of claim 36.

44. An immunogenic composition or vaccine comprising the VLP of claim 43.

45-49. (canceled)

50. A method of generating an immune response in a subject, the method comprising administering to the subject an effective amount of an immunogen generated using the method of claim 1.

51-58. (canceled)

59. A method of generating a non-naturally occurring, pan-epitopic immunogen capable of generating an immune response in a subject, the method comprising:
(a) generating a phylogenetic tree comprising full length related antigen sequences derived from one or more pathogens or pathogen strains present within a six-month time period;
(b) identifying clusters of antigen sequences within the tree, each cluster having at least 95% identity and at least about 0.001 substitution per site relative to the other sequences within the cluster;
(c) generating for each cluster a non-naturally occurring primary sequence comprising amino acids that are conserved or identical within the cluster;
(d) generating a phylogenetic tree comprising the primary sequences of step (c);
(e) selecting three or more clusters of antigen sequences within the primary sequences, each selected cluster comprising at least about 0.001 amino acid substitutions per site and generating secondary sequences comprising amino acids that are conserved or identical within the three or more clusters;
(f) repeating steps (a)-(e) until secondary sequences from a series of recent consecutive six-month time periods have been selected over a preselected total time period;
(g) generating a phylogenetic tree comprising the secondary sequences, wherein branches of the tree are combined if the substitution rate per amino acid site distance is less than about 0.001 to produce a plurality of tertiary sequences;
(h) generating a quaternary backbone sequence comprising amino acids that are conserved or identical among the tertiary sequences; and
(i) generating a non-naturally occurring, pan-epitopic immunogen by incorporating secondary sequences from step (e) into the quaternary backbone sequence, wherein:
(i) secondary sequences from the most recent six-month time period are incorporated into the backbone sequence and secondary sequences from the oldest six-month time period are eliminated from the backbone sequence, thereby producing a sequence comprising multiple secondary sequences spanning the preselected total time period; or
(ii) secondary sequences from the most recent six-month time period and from the oldest six-month time period are incorporated into the backbone sequence, thereby producing a sequence comprising multiple secondary sequences spanning the preselected total time period.

60. The method of claim 59, wherein each pathogenic strain is present in two or more selected geographic regions over one or more selected periods of time within a six-month time period.

61. The method of claim 59, wherein each influenza virus strain is present in two or more selected geographic regions over one or more flu seasons within a six-month time period.

62. The method of claim 59, wherein the full length related antigen sequences are derived from two or more H5 clades, or two or more H5 species, present in two or more selected geographic regions over one or more flu seasons within a six-month time period.

63. The method of claim 59, where the full length related antigen sequences are derived from H1 or H2 strains present during two or more consecutive flu seasons within a six-month time period.

64. A method of generating a non-naturally occurring pan-epitopic immunogen capable of generating an immune response against present and future Dengue virus strains in a subject, the method comprising:
(a) generating a phylogenetic tree comprising full length related antigen sequences derived from Dengue strains present in the Americas or Asia during a selected period of time comprising a six month time period;
(b) identifying clusters of antigen sequences within the tree, each cluster having at least 95% identity and at least about 0.001 substitution per site relative to the other sequences within the cluster;
(c) generating for each cluster a non-naturally occurring primary sequence comprising amino acids that are conserved or identical within the cluster;
(d) generating a phylogenetic tree comprising the primary sequences of step (c);
(e) selecting three or more clusters of antigen sequences within the primary sequences, each selected cluster comprising at least about 0.001 amino acid substitutions per site and generating secondary sequences comprising amino acids that are conserved or identical within the three or more clusters;
(f) repeating steps (a)-(e) until secondary sequences from a series of recent consecutive six-month time periods have been selected over a preselected total time period;
(g) generating a phylogenetic tree comprising the secondary sequences, wherein branches of the tree are combined if the substitution rate per amino acid site distance is less than about 0.001 to produce a plurality of tertiary sequences;
(h) generating a quaternary backbone sequence comprising amino acids that are conserved or identical among the tertiary sequences; and
(i) generating a non-naturally occurring, pan-epitopic immunogen by incorporating secondary sequences from step (e) into the quaternary backbone sequence, wherein:
(i) secondary sequences from the most recent six-month time period are incorporated into the backbone sequence and secondary sequences from the oldest six-month time period are eliminated from the backbone sequence, thereby producing a sequence comprising multiple secondary sequences spanning the preselected total time period; or
(ii) secondary sequences from the most recent six-month time period and from the oldest six-month time period are incorporated into the backbone sequence, thereby producing a sequence comprising multiple secondary sequences spanning the preselected total time period.

65. (canceled)


SEQUENCE LISTING (19 sequences)

SEQ ID NO: 1 - protein, 567 aa, Artificial Sequence
  Description of Artificial Sequence Synthetic polypeptide
MEKIVLLLAVVSLVKSDQICIGYHANNSTEQVDTIMEKNVTVTHAQDILEKTHNGKLCDLNGVKPLILKDCSVAGWLLGNPMCDEFLNVPEWSYIVEKANPANGLCYPGNFNDYEELKHLLSRINHFEKIQIIPKSSWSNHEASSGVSSACPYQGRSSFFRNVVWLIKKNNAYPTIKRSYNNTNQEDLLILWGIHHPNDAAEQTKLYQNPTTYVSVGTSTLNQRLVPKIATRSKVNGQSGRMEFFWTILKPNDAINFESNGNFIAPEYAYKIVKKGDSAIMKSELEYGNCNTKCQTPIGAINSSMPFHNIHPLTIGECPKYVKSNKLVLATGLRNSPQRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 2 - protein, 571 aa, Artificial Sequence
  Description of Artificial Sequence Synthetic polypeptide
MEKIVLLLAIVSLVKSDQICIGYHANNSTEQVDTIMEKNVTVTHAQDILEKTHNGKLCSLNGVKPLILKDCSVAGWLLGNPMCDEFLNVPEWSYIVEKDNPANGLCYPGNFNDYEELKHLLSSTNHFEKIQIIPRSSWSNHDASSGVSSACPYNGRSSFFRNVVWLIKKNNAYPTIKRSYNNTNQEDLLILWGIHHPNDAAEQTKLYQNPTTYVSVGTSTLNQRSVPEIATRPKVNGQSGRMEFFWTILKPNDAINFESNGNFIAPEYAYKIVKKGDSAIMKSELEYGNCNTKCQTPMGAINSSMPFHNIHPLTIGECPKYVKSBKLVLATGLRNKPQNSPQRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 3 - protein, 568 aa, Artificial Sequence
  Description of Artificial Sequence Synthetic polypeptide
MEKIVLPFAVISLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGNLNDYEELKHLLSRINHFEKTLIIPKSSWPNHETSLGVSAACPYQGTPSFFRNVVWLIKKNDAYPTIKISYNNTNREDLLILWGIHHSNNAAEQTNLYKNPTTYVSVGTSTLNQRLVPKIATRSQVNGQRGRMDFFWTILKPNDAIHFESNGNFIAPEYAYKIVKKGDSTIMKSEVEYGHCNTKCQTPIGAINSSMPFHNIHPLTIGECPKYVKSNKLVLATGLRNSPLNVPQRETRGLFGAIAGFIEGGWQGMVDGWYGYHHSNEQGSGYAADKESTQKAIDGITNKVNSIIDKMNTQFEAVGKEFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLNREEISGVKLESMGTYQILSIYSTVASSLALAIMIAGLSFWMCSNGSLQCRICI

SEQ ID NO: 4 - protein, 568 aa, Artificial Sequence
  Description of Artificial Sequence Synthetic polypeptide
MEKIVLLFAIVSLVKSDQICIGYHANNSTEQVDTIMEKNVTVTHAQDILEKTHNGKLCDLNGVKPLILKDCSVAGWLLGNPMCDEFINVPEWSYIVEKANPANDLCYPGNFNDYEELKHLLSRINHFEKIQIIPKDSWSDHEASLGVSAACPYQGNSSFFRNVVWLIKKNDAYPTIKKSYNNTNREDLLVLWGIHHPNDAAEQTRLYQNPTTYISVGTSTLNQRLVPKIATRSKVNGQSGRMDFFWTILKPNDAIHFESNGNFIAPEYAYKIVKKGDSTIMKSEVEYGNCNTKCQTPIGAINSSMPFHNIHPLTIGECPKYVKSNKLVLATGLRNSPQNVPQRETRGLFGAIAGFIEGGWQGMVDGWYGYHHSNEQGSGYAADKESTQKAIDGITNKVNSIIDKMNTQFEAVGKEFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLNREEISGVKLESMGTYQILSIYSTVASSLALAIMIAGLSFWMCSNGSLQCRICI

SEQ ID NO: 5 - protein, 571 aa, Artificial Sequence
  Description of Artificial Sequence Synthetic polypeptide
MEKIVLLLAIVSLVKSDQICIGYHANNSTEQVDTIMEKNVTVTHAQDILEKTHNGKLCSLNGVKPLILKDCSVAGWLLGNPMCDEFLNVPEWSYIVEKDNPANGLCYPGNFNDYEELKHLLSSTNHFEKIQIIPRSSWSNHDASSGVSSACPYNGRSSFFRNVVWLIKKNNAYPTIKRSYNNTNQEDLLILWGIHHPNDAAEQTKLYQNPTTYVSVGTSTLNQRSVPEIATRPKVNGQSGRMEFFWTILKPNDAINFESNGNFIAPEYAYKIVKKGDSAIMKSELEYGNCNTKCQTPVGAINSSMPFHNIHPLTIGECPKYVKSNKLVLATGLRNIPQNSPQRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 6 - protein, 571 aa, Artificial Sequence
  Description of Artificial Sequence Synthetic polypeptide
MEKIVLLFATISLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHLLSRINHFEKTLIIPKSSWPNHETSLGVSAACPYQGTPSFFRNVVWLIKKNDAYPTIKISYNNTNREDLLILWGIHHSNNAAEQTNLYKNPTTYVSVGTSTLNQRLVPKIATRSQVNGQRGRMDFFWTILKPNDAIHFESNGNFIAPEYAYKIVKKGDSTIMKSEMEYGHCNTKCQTPIGAINSSMPFHNIHPLTIGECPKYVKSNKLVLATGLRNSPLNSPQRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 7 - protein, 571 aa, Artificial Sequence
  Description of Artificial Sequence Synthetic polypeptide
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGNLNDYEELKHLLSRINHFEKILIIPKSSWPNHETSLGVSAACPYQGTPSFFRNVVWLIKKNDAYPTIKISYNNTNREDLLILWGIHHSNNAAEQTNLYKNPTTYISVGTSTLNQRLVPKIATRSQVNGQRGRMDFFWTILKPNDAIHFESNGNFIAPEYAYKIVKKGDSTIMKSEVEYGHCNTKCQTPIGAINSSMPFHNIHPLTIGECPKYVKSNKLVLATGLRNSPLNSPQRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLKDNAKELGNGCFEFYHKCNNECMESVRNGTYDYPQYSEEARLKREEISGVKLESIGIYQILSIYSTVASSLVLAIMMAGLSLWMCSNGSLQCRICI

SEQ ID NO: 8 - protein, 571 aa, Artificial Sequence
  Description of Artificial Sequence Synthetic polypeptide
MEKIVLLFAIVSLVKSDQICIGYHANNSTEQVDTIMEKNVTVTHAQDILEKTHNGKLCDLNGVKPLILKDCSVAGWLLGNPMCDEFINVPEWSYIVEKANPANDLCYPGNFNDYEELKHLLSRINHFEKIQIIPKSSWSDHEASLGVSAACPYQGNSSFFRNVVWLIKKNNAYPTIKKSYNNTNQEDLLVLWGIHHPNDAAEQTRLYQNPTTYISVGTSTLNQRLVPKIATRSKVNGQSGRMDFFWTILKPNDAIHFESNGNFIAPEYAYKIVKKGDSTIMKSELEYGNCNTKCQTPIGAINSSMPFHNIHPLTIGECPKYVKSNKLVLATGLRNSPQNSPQRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLKDNAKELGNGCFEFYHKCNNECMESVRNGTYDYPQYSEEARLKREEISGVKLESIGIYQILSIYSTVASSLVLAIMMAGLSLWMCSNGSLQCRICI

SEQ ID NO: 9 - protein, 470 aa, Artificial Sequence
  Description of Artificial Sequence Synthetic polypeptide
MNPNQKIITIGSISIAIGIISLMLQIGNIISIWASHSIQTGSQNNTGICNQRIITYENSTWVNHTYVNINNTNVVAGQDKTSVTLAGNSSLCSISGWAIYSKDNSIRIGSKGDVFVIREPFISCSHLECRTFFLTQGALLNDKHSNGTVKDRSPYRALMSCPLGEAPSPYNSRFESVAWSASACHDGMGWLTIGISGPDNGAVAVLKYNGIITDTIKSWKKQILRTQESECVCMNGSCFTIMTDGPSNGAASYKIFKIEKGKVTKSVELNAPNFHYEECSCYPDTGIVMCVCRDNWHGSNRPWVSFNQNLDYQIGYICSGVFGDNPRPNDGEGSCNPVSSNGANGVKGFSYKYGNGVWIGRTKSNRLRKGFEMIWDPNGWTGTDSDFSVKQDVVAITDWSGYSGSFVQHPELTGLDCIRPCFWVELIRGLPKENTTIWTSGSSISFCGVNSDTANWSWPDGAELPFTIDK

SEQ ID NO: 10 - protein, 565 aa, Influenza virus
MEARLLVLLCAFAATNADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYANNKEKEVLVLWGVHHPPTSTDQQSLYQNENAYVSVVSSHYSRKFTPEIAKRPKVRDQEGRINYYWTLLEPGDTIIFEANGNLIAPRYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 11 - protein, 562 aa, Artificial Sequence
  Description of Artificial Sequence Synthetic polypeptide
MAIIYLILLFTAVRGDQICIGYHANNSTEKVDTILERNVTVTHAKDILEKTHNGKLCKLNGIPPLELGDCSIAGWLLGNPECDRLLSVPEWSYIMEKENPRNGLCYPGSFNDYEELKHLLSSVTHFEKVKILPKDRWTQHTTTGGSRACAVSGNPSFFRNMVWLTKKGSNYPVAKGSYNNTSGEQMLIIWGVHHPNDEAEQRTLYQNVGTYVSVGTSTLNKRSIPEIATRPKVNGQGGRMEFSWTLLDMWDTINFESTGNLIAPEYGFKISKRGSSGIMKTEGTLENCETKCQTPLGAINTTLPFHNIHPLTIGECPKYVKSERLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVKNLYDKVRMQLRDNAKELGNGCFEFYHKCDDECMNSVKNGTYDYPKYEEESKLNRNEIKGVKLSNMGVYQILAIYATVAGSLSLAIMIAGISFWMCSNGSLQCRICI

SEQ ID NO: 12 - protein, 562 aa, Artificial Sequence
  Description of Artificial Sequence Synthetic polypeptide
MAIIYLILLFTAVRGDQICIGYHSNNSTEKVDTILERNVTVTHAQDILEKTHNGKLCKLNGIPPLELGDCSIAGWLLGNPECDRLLTVPEWSYIMEKENPRNGLCYPGSFNDYEELKHLLSSVTHFEKVKILPKDRWTQHTTTGGSRACAVSGNPSFFRNMVWLTKKGSNYPIAKGSYNNTSGEQMLIIWGVHHPNDEAEQRTLYQNVGTYVSVGTSTLNKRSIPEIATRPKVNGQGGRMEFSWTILDMLDTINFESTGNLIAPEYGFKISKRGSSGIMKTEGTLENCETKCQTPLGAINTTLPFHNIHPLTIGECPKYVKSERLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVKNLYDKVRMQLRDNAKELGNGCFEFYHKCDDECMNSVKNGTYDYPKYEEESKLNRNEIKGVKLSNMGVYQILAIYATVAGSLSLAIMIAGISFWMCSNGSLQCRICI

SEQ ID NO: 13 - protein, 562 aa, Artificial Sequence
  Description of Artificial Sequence Synthetic polypeptide
MAIIYLILLFTAVRGDQICIGYHSNNSTEKVDTILERNVTVTHAQDILEKTHNGKLCKLNGIPPLELGDCSIAGWLLGNPECDRLLTVPEWSYIMEKENPRNGLCYPGSFNDYEELKHLLSSVTHFEKVKILPKDRWTQHTTTGGSRACAVSGNPSFFRNMVWLTKKGSNYPIAKGSYNNTSGEQMLIIWGVHHPNDEAEQRTLYQNVGTYVSVGTSTLNKRSIPEIATRPKVNGQGGRMEFSWTILDIWDTINFESTGNLIAPEYGFKISKRGSSGIMKTEGTLENCETKCQTPLGAINTTLPFHNIHPLTIGECPKYVKSERLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVKNLYDKVRMQLRDNAKELGNGCFEFYHKCDDECMNSVKNGTYDYPKYEEESKLNRNEIKGVKLSNMGVYQILAIYATVAGSLSLAIMIAGISFWMCSNGSLQCRICI

SEQ ID NO: 14 - protein, 562 aa, Artificial Sequence
  Description of Artificial Sequence Synthetic polypeptide
MAIIYLILLFTAVRGDQICIGYHSNNSTEKVDTILERNVTVTHAQDILEKTHNGKLCKLNGIPPLELGDCSIAGWLLGNPECDRLLTVPEWSYIMEKENPRNGLCYPGSFNDYEELKHLLSSVTHFEKVKILPKDRWTQHTTTGGSRACAVSGNPSFFRNMVWLTKKGSNYPVAKGSYNNTSGEQMLIIWGVHHPNDETEQRTLYQNVGTYVSVGTSTLNKRSIPEIATRPKVNGQGGRMEFSWTILDMLDTINFESTGNLIAPEYGFKISKRGSSGIMKTEGTLENCETKCQTPLGAINTTLPFHNIHPLTIGECPKYVKSERLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVKNLYDKVRMQLRDNAKELGNGCFEFYHKCDDECMNSVKNGTYDYPKYEEESKLNRNEIKGVKLSNMGVYQILAIYATVAGSLSLAIMIAGISFWMCSNGSLQCRICI

SEQ ID NO: 15 - protein, 562 aa, Artificial Sequence
  Description of Artificial Sequence Synthetic polypeptide
MAIIYLILLFTAVRGDQICIGYHANNSTEKVDTILERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKKGSNYPVAKGSYNNTSGEQMLIIWGVHHPNDETEQRTLYQNVGTYVSVGTSTLNKRSTPEIATRPKVNGQGGRMEFSWTLLDMWDTINFESTGNLIAPEYGFKISKRGSSGIMKTEGTLENCETKCQTPLGAINTTLPFHNIHPLTIGECPKYVKSERLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVKNLYDKVRMQLRDNAKELGNGCFEFYHKCDDECMNSVKNGTYDYPKYEEESKLNRNEIKGVKLSNMGVYQILAIYATVAGSLSLAIMIAGISFWMCSNGSLQCRICI

SEQ ID NO: 16 - protein, 562 aa, Artificial Sequence
  Description of Artificial Sequence Synthetic polypeptide
MAIIYLILLFTAVRGDQICIGYHANNSTEKVDTILERNVTVTHAKDILEKTHNGKLCKLNGIPPLELGDCSIAGWLLGNPECDRLLRVPEWSYIMEKENPRYSLCYPGSFNDYEELKHLLSSVKHFEKVKILPKDRWTQHTTTGGSRACAVSGNPSFFRNMVWLTKKGPNYPVAKGSYNNTSGEQMLIIWGVHHPNDEAEQRALYQNVGTYVSASTSTLNKRSTPEIAARPKVNGLGSRMEFSWTLLDMWDTINFESTGNLVAPEYGFKISKRGSSGIMKTEGTLENCETKCQTPLGAINTTLPFHNIHPLTIGECPKYVKSERLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVKNLYDKVRMQLRDNAKELGNGCFEFYHKCDDECMNSVKNGTYDYPKYEEESKLNRNEIKGVKLSNMGVYQILAIYATVAGSLSLAIMIAGISFWMCSNGSLQCRICI

SEQ ID NO: 17 - protein, 562 aa, Artificial Sequence
  Description of Artificial Sequence Synthetic polypeptide
MAIIYLILLFTAVRGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKVKILPRDQWTQHTTTGGSRACAVSGNPSFFRNMVWLTKKGSNYPIAKRSYNNTSGEQMLIIWGIHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETKCQTPLGAINTTLPFHNIHPLTIGECPKYVKSERLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVKNLYDKVRMQLRDNAKELGNGCFEFYHKCDDECMNSVKNGTYDYPKYEEESKLNRNEIKGVKLSNMGVYQILAIYATVAGSLSLAIMIAGISFWMCSNGSLQCRICI

SEQ ID NO: 18 - protein, 562 aa, Artificial Sequence
  Description of Artificial Sequence Synthetic polypeptide
MAIIYLILLFTAVRGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKIKILPRDQWTQHTTTGGSRACAVSGNPSFFRNMVWLTKKGSNYPIAKRSYNNTSGEQMLIIWGIHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETKCQTPLGAINTTLPFHNIHPLTIGECPKYVKSERLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVKNLYDKVRMQLRDNAKELGNGCFEFYHKCDDECMNSVKNGTYDYPKYEEESKLNRNEIKGVKLSNMGVYQILAIYATVAGSLSLAIMIAGISFWMCSNGSLQCRICI

SEQ ID NO: 19 - protein, 562 aa, Artificial Sequence
  Description of Artificial Sequence Synthetic polypeptide
MAIIYLILLFTAVRGDQICIGYHANNSTEKVDTILERNVTVTHAKDILEKTHNGKLCRLSGIPPLELGDCSIAGWLLGNPECDRLLSVPEWSYIVEKENPVNGLCYPGSFNDYEELKHLLTSVTHFEKIKILPRDQWTQHTTTGGSRACAVSGNPSFFRNMVWLTKKGSNYPVAKRSYNNTSGEQMLIIWGIHHPNDDAEQRTLYQNVGTYVSVGTSTLNKRSIPEIATRPKVNGQGGRMEFSWTLLETWDVINFESTGNLIAPEYGFKISKRGSSGIMKTEKTLENCETKCQTPLGAINTTLPFHNIHPLTIGECPKYVKSERLVLATGLRNVPQIESRGLFGAIAGFIEGGWQGMVDGWYGYHHSNDQGSGYAADKESTQKAIDGITNKVNSVIEKMNTQFEAVGKEFNNLERRLENLNKKMEDGFLDVWTYNAELLVLMENERTLDFHDSNVKNLYDKVRMQLRDNAKELGNGCFEFYHKCDDECMNSVKNGTYDYPKYEEESKLNRNEIKGVKLSNMGVYQILAIYATVAGSLSLAIMIAGISFWMCSNGSLQCRICI